(19) United States
(12) Patent Application Publication
(10) Pub. No.: US 2007/0042494 A1
(43) Pub. Date: Feb. 22, 2007

(54) Heterologous retroviral packaging system

(76) Inventors: Tal Kafri, Carrboro, NC (US); Hong Ma, Morrisville, NC (US); Adam Cockrell, Durham, NC (US)

Correspondence Address: JENKINS, WILSON, TAYLOR & HUNT, P. A., 3100 TOWER BLVD, SUITE 1200, DURHAM, NC, 27707, US

(21) Appl. No.: 11/444,523

(22) Filed: May 31, 2006

Related U.S. Application Data
(60) Provisional application No. 60/685,824, filed on May 31, 2005.

(51) Int. Cl. C12N 15/867; C12N 7/00; C12N 5/06

(52) U.S. Cl. 435/456; 435/235.1; 977/802; 435/325

(57) ABSTRACT

A chimeric retroviral vector comprising sequences from at least two retroviruses, wherein at least one of the sequences encodes a cis element, and wherein the chimeric retroviral vector is capable of being packaged in a viral particle; and methods of making and using the same.

### CROSS REFERENCE TO RELATED APPLICATIONS

This application is based on and claims priority to U.S. Provisional Application Ser. No. 60/685,824, filed May 31, 2005, herein incorporated by reference in its entirety.

### GRANT STATEMENT

These studies were supported by Grant Nos. POI DK 058702 and POI HL 066973. As such, the U.S. Government has certain rights in the presently disclosed subject matter.

### TECHNICAL FIELD

The presently disclosed subject matter generally relates to the development of an improved retroviral vector system that affords efficient cross-packaging of heterologous retroviral genetic elements.

TABLE OF ABBREVIATIONS

| | |
|---|---|
| Ab | antibody |
| APC | antigen-presenting cell |
| aPTT | Activated Partial Thromboplastin Time |
| 5-AzaC | 5-azacytidine |
| AZT | azidothymidine |
| bp | base pairs |
| cDNA | complementary DNA |
| CMV | cytomegalovirus |
| cPPT | central polypurine tract |
| CTE | constitutive transport element |
| DC | dendritic cells |
| DNA | deoxyribonucleic acid |
| DNase | deoxyribonuclease |
| EIAV | equine infectious anemia virus |
| ELISA | Enzyme-Linked Immunosorbent Assay |
| FACS | fluorescence-activated cell sorting |
| FIV | feline immunodeficiency virus |
| FIX | factor IX |
| g | gravity |
| GAPDH | glyceraldehyde-3-phosphate dehydrogenase |
| GEF | guanine nucleotide exchange factor |
| GFP | green fluorescent protein |
| h | hour |
| hAAT | human alpha-1 antitrypsin |
| HDAC | histone deacetylase |
| HEF | human embryonic fibroblast |
| hFIX | human factor IX |
| HIV-1 | human immunodeficiency virus type 1 |
| HMEC | human mammal epithelial cell |
| HR | homologous recombination |
| HRP | horseradish peroxidase |
| INS | instability sequence |
| IP | intraperitoneal |
| IRES | internal ribosome entry site |
| IU | international unit |
| kb | kilobase |
| KO | knock out |
| LAM-PCR | linear amplification-mediated PCR |
| LCR | locus control region |
| LTR | long terminal repeat |
| MBDs | methyl domain binding protein |
| MFI | mean fluorescence intensity |
| mg | milligram |
| min | minute |
| ml | milliliter |
| MLV | murine leukemia virus |
| mM | millimolar |
| MNase | micrococcal nuclease |
| MOI | multiplicity of infection |
| mol | mole |
| mRNA | messenger RNA |
| ng | nanogram |
| NHEJ | nonhomologous end-joining |
| nM | nanomolar |
| NRF | nuclear respiratory factor |
| ³²P | Phosphorous-32 |
| PBS | phosphate buffered saline |
| pcDNA | packaging construct DNA |
| PCR | polymerase chain reaction |
| PolyA | polyadenylation |
| PP2A | protein phosphatase 2A |
| qPCR | quantitative PCR |
| RCR | replication competent retrovirus |
| RNA | ribonucleic acid |
| RRE | Rev response element |
| RT | reverse transcriptase |
| SB | sodium butyrate |
| SIN | self-inactivating |
| TAA | tumor-associated antigen |
| TSA | trichostatin A |
| tTA | tetracycline transactivator |
| μg | microgram |
| μl | microliter |
| UTR | untranslated region |
| μM | micromolar |
| VPA | valproic acid |
| VSV-G | vesicular stomatitis virus glycoprotein |
| WPRE | woodchuck hepatitis virus posttranscriptional |
| | regulatory element |
| Wt | wild type |
| % | percent |
| ° C. | degrees Celsius |
| ≧ | greater than or equal to |
| > | greater than |
| ≦ | less than or equal to |
| < | less than |

### BACKGROUND

The capacity to introduce a particular foreign or native gene sequence into a cell and to control the expression of that gene is of value in the fields of medical and biological research. Such capacity has a wide variety of useful applications, including but not limited to studying gene regulation and designing a therapeutic basis for the treatment of disease.

The introduction of a particular foreign or native gene into a host cell is facilitated by introducing a gene sequence into a suitable nucleic acid vector. A variety of methods have been developed that allow the introduction of such a recombinant vector into a desired host cell. The use of viral vectors can result in the rapid introduction of the recombinant molecule into a wide variety of host cells.

Retroviruses are RNA viruses that replicate through a DNA proviral intermediate that is usually integrated in the genome of the infected host cell. All known retroviruses share features of the replicative cycle, including packaging of viral RNA into virions, entry into target cells, reverse transcription of viral RNA to form the DNA provirus, and stable integration of the provirus into the target cell genome. Replication competent proviruses typically comprise regulatory long terminal repeats (LTRs) and the gag, pro, pol and env genes which encode core proteins, a protease, reverse transcriptase/RNAse H/integrase and envelope glycoproteins, respectively.

Retroviral vectors are a common tool for gene delivery in that the ability of retroviral vectors to deliver an unrearranged, single copy gene into a broad range of cells makes them well suited for transferring genes to a cell. While recombinant retroviral vectors allow for integration of a transgene into a host cell genome, most retroviruses can only transduce dividing cells. This can limit their use for in vivo gene transfer to nonproliferating cells such as hepatocytes, myofibers, hematopoietic stem cells, and neurons. Non-dividing cells are the predominant, long-lived cell type in the body, and account for most desirable targets of gene transfer, including liver, muscle, and brain.

Lentiviruses are a subgroup of retroviruses that are capable of infecting non-dividing cells. These viruses include, but are not limited to, HIV-1, EIAV, and FIV. Like other retroviruses, lentiviruses possess gag, pol and env genes that are flanked by two long terminal repeat (LTR) sequences. Each of these genes encodes multiple proteins, initially expressed as one precursor polyprotein. The gag gene encodes the internal structural (matrix capsid and nucleocapsid) proteins. The pol gene encodes the RNA-directed DNA polymerase (reverse transcriptase, integrase and protease). The env gene encodes viral envelope glycoproteins and additionally contains a cis-acting element (RRE) responsible for nuclear export of viral RNA. Gene transfer systems based on lentiviruses have emerged as promising gene delivery vehicles for human gene therapy due to their ability to efficiently transduce nondividing target cells.

Human immunodeficiency virus (HIV) and all other lentiviruses utilize the essential viral protein Rev, which binds to RRE RNA, to export unspliced and partially spliced mRNAs from the nucleus. RNA and incompletely spliced mRNA must be exported to the cytoplasm for packaging or translation. This process is mediated by the trans-acting viral protein Rev in concert with its response element (RRE).

The risk of an inadvertent transfer of viral genes encoding genetic material into target cells in the course of a gene therapy protocol can be a bio-safety concern. In the worst-case scenario, such an event can result in the emergence of a replication competent retrovirus (RCR). Another problem in the art is the potential for vector-induced insertional mutagenesis.

Accordingly, the development of improved vector systems capable of mediating gene transfer into a broad range of dividing and non-dividing cells remains a need in the art.

### SUMMARY

Disclosed here are methods of producing chimeric vector particles, wherein a first retroviral vector is packaged into a second retroviral vector particle, the method comprising (a) cloning a nucleic acid sequence encoding a second retroviral cis element into the first retroviral vector RNA to generate a chimeric vector; and (b) transfecting a packaging cell line with said chimeric vector, wherein packaging cell line provides proteins for the retroviral vector to be packaged.

Also disclosed herein are chimeric retroviral vectors comprising sequences from at least two retroviruses, wherein at least one of the sequences encodes a cis element that provides promiscuous packaging of the retroviral vector.

Further disclosed herein are producer cell lines for producing retroviral particles, the producer cell comprising a retroviral vector and DNA constructs coding for proteins required for the retroviral vector to be packaged, said retroviral vector comprising in 5′ to 3′ order: (a) a 5′ long terminal repeat (LTR) from a first retrovirus; (b) a sequence encoding a second retrovirus Rev Response Element (RRE); and (c) a 3′ long terminal repeat (LTR) from the first retrovirus, wherein the chimeric retroviral vector is capable of being packaged in a viral particle of the second retrovirus.

Also disclosed is a retroviral vector kit comprising: (a) a retroviral vector which comprising, in 5′ to 3′ order; (i) a 5′ long terminal repeat (LTR) from a first retrovirus; (ii) a sequence encoding a second retrovirus Rev Response Element (RRE); and (iii) a 3′ long terminal repeat region from the first retrovirus, wherein the chimeric retroviral vector is capable of being packaged in a viral particle of the second retrovirus; and (b) a packaging cell line comprising at least one retroviral or recombinant retroviral construct coding for proteins required for said retroviral vector to be packaged.

Also disclosed herein are recombinant retroviral particles comprising the disclosed retroviral vectors.

In some embodiments, the chimeric retroviral vector comprises a 5′ long terminal repeat (LTR) from a first retrovirus.

In some embodiments, the first retroviral vector comprises a lentivirus. In some embodiments, the lentivirus is selected from the group consisting of FIV, EIAV, and MLV.

In some embodiments, the second retroviral vector cis element is selected from the group consisting of a RRE, an Env gene fragment from the region flanking the RRE, and cPPT. In some embodiments, the Env gene fragment from the region flanking RRE is about 140 bp 5′ of the RRE and about 475 bp 3′ of the RRE.

In some embodiments, the first retrovirus is a non-HIV-1 retrovirus and the second retrovirus is a HIV-1 retrovirus.

In some embodiments, each long terminal repeat region is derived from a retrovirus selected from the group selected from the group consisting of Murine Leukemia Virus, Mouse Mammary Tumor Virus, Murine Sarcoma Virus, Simian Immunodeficiency Virus, Human T Cell Leukemia Virus, Feline Immunodeficiency Virus, Feline Leukemia Virus, Bovine Leukemia Virus, and Mason-Pfizer-Monkey Virus.

In some embodiments, the one or more HIV-1 envelope sequences are oriented between the 5′ LTR and the 3′ LTR.

In some embodiments, the one or more HIV-1 envelope sequences flank the RRE sequence 5′, 3′, or both 5′ and 3′.

In some embodiments, the retroviral vector further comprises a HIV-1 cPPT sequence oriented between the 5′ LTR and 3′ LTR.

In some embodiments, the cPPT sequence flanks the RRE sequence 3′.

In some embodiments, the Env gene fragment from the region flanking HIV-1 RRE is about 140 bp 5′ of the RRE and about 475 bp 3′ of the RRE.

In some embodiments, the retroviral vector further comprises one or more coding sequences operably linked to a heterologous promoter.

In some embodiments, the coding sequences are selected from the group consisting of marker genes, therapeutic genes, antiviral genes, antitumor genes, cytokine genes, genes encoding antigens, and combinations thereof.

In some embodiments, the marker or therapeutic genes are selected from the group consisting of β-galactosidase gene, neomycin gene, puromycin gene, cytosine deaminase gene, secreted alkaline phosphatase gene, and combinations thereof.

In some embodiments, the retroviral vector comprises a heterologous promoter oriented 5′ to the 5′ LTR.

In some embodiments, the heterologous promoters are the same or different.

In some embodiments, a composition comprises the recombinant retroviral particles and a pharmaceutically acceptable carrier.

In some embodiments, a retroviral provirus is produced by infection of target cells with a recombinant retroviral particle.

In some embodiments, the mRNA of the retroviral provirus is disclosed.

In some embodiments, the RNA of a retroviral vector is disclosed.

In some embodiments, the packaging cell line harbors retroviral or recombinant retroviral constructs coding for those retroviral proteins which are not encoded in said retroviral vector.

In some embodiments, the packaging cell line is selected from the group consisting of SODk-1, WAN-1, or SODk-3.

In some embodiments, a recombinant particle is used to introduce homologous or heterologous nucleotide sequences into cells in an animal or cultured cells, said method comprising infecting the cells.

### BRIEF DESCRIPTION OF THE DRAWINGS

FIG. 1 is a series of schematic diagrams of the vectors constructed in accordance with the presently disclosed subject matter. Abbreviations used in the figure include: CMV—cytomegalovirus promoter; ψ—packaging signal; Env—HIV-1 envelope; RRE—HIV-1 Rev response element; cPPT—central polypurine tract; GFP—green fluorescent protein; WP—woodchuck hepatitis virus posttranscriptional regulatory element; Δu3—self-inactivating deletion in U3; FIX—human factor IX cDNA; GFP/Cre—fused genes; hAAT—human a-1 antitrypsin promoter; Ind Pr—Tetracycline inducible promoter.

FIG. 2 is a series of immunofluorescence counts representing the MLV vector TK493, chimeric HIV-1/MLV vector TK494, traditional EIAV vector UNC 6.1, chimeric vector pTK728, TK665 (−HIV-1 RRE), and TK660 (+HIV-1 RRE) packaged either with the parental EIAV or the HIV-1 packaging system. Vector particles were generated by transfection of 293T cells and GFP expression was determined by FACS analysis and fluorescence microscopy at day 5 or after 3-5 passages in culture.

FIG. 3 is a series of immunofluorescence counts representing the HIV-1 vector vTK113 packaged with the HIV-1 packaging cassette, vector chimeric HIV-1/MLV vector TK494 packaged with either the HIV-1 or MLV packaging cassette, and MLV vector TK506 packaged with the MLV packaging cassette. The vector particles were collected from conditioned media and used to transduce 293T cells in either the presence or absence of reverse-transcriptase inhibitors AZT (50 mM) and nevirapine 1 μg/mL). AZT is a nucleoside analog inhibitor of reverse transcription, thus its activity is not limited to specific retroviruses. Nevirapine is a non-nucleoside inhibitor whose activity requires interaction with the HIV-1 reverse transcriptase, thus it is not expected to significantly inhibit transduction.

FIG. 4 is a schematic representation of vectors vTK660S, vTK660M, and vTK660L. Abbreviations used in the figure include: CMV—cytomegalovirus promoter; R—the repeat sequence at the 5′ and 3′ ends of the viral/vector full-length RNA; U5—U5 LTR; ψ—packaging signal; Env—HIV-1 envelope; RRE—HIV-1 Rev response element; cPPT—central polypurine tract; GFP—green fluorescent protein; WP—woodchuck hepatitis virus posttranscriptional regulatory element; U3—U3 LTR.

FIG. 5 is a schematic representation of pMLVΔψ RRE and pMLVΔψ, constructed by amplifying a region of the MLV dimerization domain 5′ to the MLV ψ signal. Abbreviations used in the figure include: CMV—cytomegalovirus promoter; R—the repeat sequence at the 5′ and 3′ ends of the viral/vector full-length RNA; U5—U5 LTR; Δψ—deletion in packaging signal; Env—HIV-1 envelope; RRE—HIV-1 Rev response element; cPPT—central polypurine tract; GFP—green fluorescent protein; WP—woodchuck hepatitis virus posttranscriptional regulatory element; Δu3—self-inactivating deletion in U3.

FIG. 6 is a set of autoradiographs from Western blots demonstrating induction of the WAN-1 cell line. Lanes A, B, C contain protein extracted from non-induced cells, induced cells, cells induced in the presence of 5 mM sodium butyrate, respectively. Lane D contains protein extracted from vector particles.

FIG. 7 is a set of autoradiographs from Western blots demonstrating induction of VSV-G, Gag and HIV-1 RT (Pol). Lanes A, B, C, D, and E contain protein extracted from vector particles generated by transient transfection, vector particles generated by the SODk-3 cells, non-induced cells, induced cells, and 293T cells, respectively.

FIG. 8 is a schematic representation of integrated vTK731, an IRES-GFP containing conditional SIN vector. Abbreviations used in the figure include: Tet-Ind—Tetracycline inducible promoter; R—the repeat sequence at the 5′ and 3′ ends of the viral/vector full-length RNA; U5—U5 LTR; ψ—packaging signal; cPPT—central polypurine tract; CMV—cytomegalovirus promoter; RFP—red fluorescence protein; IRES—internal ribosome entry site; GFP—green fluorescent protein.

FIG. 9 is a series of immunofluorescence counts indicating sorting of SODk-3 cells transduced with vTK731. The parental, pre-sorted population and its titers is on top. GFP expression of the sorted cell populations and the titers obtained from fractions 1-4 are indicated.

FIG. 10 is a slot blot of vector RNA content in identical amounts (p24, normalized) of vector particles obtained from the parental population (A), and from fractions 1-4 (B-E).

FIG. 11 is a schematic representation of the viral structure of traditional HIV-1 pTK113, non-SIN, and SIN MLV vectors pTK506, and pTK493, respectively, and the chimeric vector pTK494. Abbreviations used in the figure include: CMV—cytomegalovirus promoter; R—the repeat sequence at the 5′ and 3′ ends of the viral/vector full-length RNA; U5—U5 LTR; ψ—packaging signal; Env—HIV-1 envelope; RRE—HIV-1 Rev response element; cPPT—central polypurine tract; GFP—green fluorescent protein; WP—woodchuck hepatitis virus posttranscriptional regulatory element; Δu3—self-inactivating deletion in U3.

FIG. 12 is a set of images and photos showing GFP expression determined by FACS analysis at day 5 post transduction. Traditional MLV, vTK493, or RRE containing chimeric vector vTK494 were packaged either with parental MLV or HIV-1 packaging system.

FIG. 13 is a Western blot indicating that deletions in the packaging signal significantly reduced the titers of the MLV-packaged vectors. A is HIV-1 vector vTK113, B is MLV vector vTK493, C is chimeric vector vTK494, D is chimeric vector vTK797, E is chimeric vector vTK802. 1 indicates the HIV-1 packaging system, 2 indicates the same as 1 with five-fold less particles loaded, 3 is MLV packaging system, 4 is same as 3, but with five-fold less particles loaded, 5 and 6 are titers of the vectors in IU/mL upon packaging with HIV, and MLV packaging systems.

FIG. 14 is a series of images representing LacZ staining of 293-LoxP-stop-LoxP-LacZ cells following transduction with packaging signal containing chimeric vector vTK631 (A, C) or with packaging signal deleted chimeric vector vTK816 (B, D). Both vectors expressed the Cre-GFP fusion protein and were packaged by either MLV (A, B) or HIV-1 packaging system (C, D).

FIG. 15 is a series of dot blots of RNA extracted from conditioned media containing vector particles using a ³²P labeled probe directed to the HIV-1 pol gene. A is RNA extracted from vTK493 (MLV packaged). B is RNA extracted from vTK493 (HIV-1 packaged). C is RNA extracted from vTK494 (MLV packaged). D is RNA extracted from vTK493 (HIV-1 packaged). E is RNA extracted from vTK113 (HIV-1 packaged). Vector particles were pelleted from either 0.1 (1) or 0.2 (2) mL of conditioned media.

FIG. 16 is a schematic representation of the traditional EIAV UNC6.1 and the chimeric EIAV/HIV-1 vector pTK728. Abbreviations in the Figure include: CMV—cytomegalovirus promoter; R—the repeat sequence at the 5′ and 3′ ends of the viral/vector full-length RNA; U5—U5 LTR; ψ Env—packaging signal envelope; RRE—HIV-1 Rev response element; Env—HIV-1 envelope; cPPT—central polypurine tract; GFP—green fluorescent protein; WP—woodchuck hepatitis virus posttranscriptional regulatory element; Δu3—self-inactivating deletion in U3.

FIG. 17 is series of images and plots showing fluorescence microscopy and FACS analysis of GFP expression of EIAV vector UNC 6.1 and chimeric vector pTK728 packaged with either parental EIAV or HIV-1 packaging system at day 5 or after 3-5 passages in culture.

FIG. 18 is an autoradiograph of a Southern blot showing DNA extracted from cells transduced with either UNC6.1 (lanes C, D, G, H) or vTK728 (lanes A, B, E, F) packaged with either EIAV (A, B, C, D) or HIV-1 (E, F, G, H). DNA was extracted at day 5 (lanes A, C, E, G) or day 14 (lanes B, D, F, H) post-transduction. The DNA was hybridized with a probe directed to recognize DNA derived from all vector forms (1) or specific to linear (3) or to single LTR circle form (2).

FIG. 19 is a series of plots of GFP expression as determined by FACscan analysis of 293T cells transduced at low MOI (less than 0.3), with vTK725 packaged by either traditional HIV-1 particles (B) or by integrase deficient particles (C). Nontransduced cells served as control (A).

FIG. 20 is set of photographs showing firefly luciferase expression at 3 (B, C) and 23 (B′, C′) weeks post-IP injection of HIV-fvTK857 packaged into HIV-1 particles. PBS injected mice (A, A′) served as controls.

FIG. 21A shows ethidium bromide staining of DNA from MNasel-digested nuclei electrophoresed in 2% agarose gels.

FIG. 21B is a Southern blot of DNA from MNasel-digested nuclei electrophoresed in 2% agarose gels. 293T cells were transduced with either a traditional or integrase mutant HIV-1 vector (lanes 1, 3 and 2, 4, respectively). Nuclei were isolated from cells 72 h (lanes 1, 2) and 10 days (lanes 3, 4) after infection and digested with MNase I (concentration 5, 1.25, and 0 U/mL). The digested DNA was hybridized with a ³²P-labeled probe encompassing the CMV promoter and GFP encoding regions of the pTK 113 construct. Linker sites cleaved by MNase I are indicated by arrows.

FIG. 22A is a schematic representation of HIV-1 vector structure. The EcoRI site and the probe used for hybridization are indicated.

FIG. 22B is an autoradiogram of nuclei digested with increasing concentrations of DNAsel followed by EcoRI digestion. On the left, integrated vector genome extracted from cells transduced with a traditional vector, followed by 5 passages. On the right, episomal vector genome extracted from cells transduced with an integrase mutant vector.

FIG. 23A is a schematic representation of the location of probe and restriction sites EcoNI, XhoI, and AatII.

FIG. 23B is an autoradiogram of a Southern blot of HEFs transduced with either traditional (2, 3) or integrase deficient vTK113 (4, 5, 6). DNA was extracted at days 3 (4), (14 (3, 5), 20 (2, 6), or following 5 passages in culture. Lane 7 served as control for methylated DNA.

FIG. 24A is a photograph showing ethidium bromide staining of DNA extracted from HEFs transduced with traditional (3) or integrase mutant (1, 2, 4) vTK113 at day 3(1), 20 (2, 3, 4). Cells were either passaged (3, 4) or not (1, 2). Digested DNA was electrophoresed in 1% gel.

FIG. 24B is an autoradiogram of DNA extracted from HEFs transduced with traditional (3) or integrase mutant (1, 2, 4) vTK113 at day 3(1), 20 (2, 3, 4). Cells were either passaged (3, 4) or not (1, 2). Digested DNA was electrophoresed in 1% gel.

FIG. 25 is a schematic representation of pIShGP and pCShGP. The abbreviations included in the Figure include: Inducible Prom—inducible promoter; hGag/Pol—humanized Gag/Pol sequences; SV40 pA—SV40 polyadenylation signal; SV40 Prom—SV40 promoter; Neo—neomycin; pA—polyadenylation; Amp—ampicillin; Ori—origin of replication.

FIG. 26 is a schematic representation of pBIGFV. Abbreviations used in the Figure include: VSV-G—vesticular stomatitis virus G protein; β-Globin pA—beta-globin polyadenylation site; ColE1 Ori—E. coli origin of replication; Amp—ampicillin; SV40 pA—SV40 polyadenylation signal; GFP—green fluorescent protein.

FIG. 27 is a series of schematic representations of vTK790, vTK789, and vTK136. Abbreviations used in the Figure include: CMV—cytomegalovirus promoter; R—the repeat sequence at the 5′ and 3′ ends of the viral/vector full-length RNA; U5—U5 LTR; ψ—packaging signal; Env—HIV-1 envelope; RRE—HIV-1 Rev response element; cPPT—central polypurine tract; hAAT—human a-1 antitrypsin promoter; GFP—green fluorescent protein; WP—woodchuck hepatitis virus posttranscriptional regulatory element; Ind Pr—Tetracycline inducible promoter.

FIG. 28 is an autoradiograph of a Southern blot of the 293-F113 cell line. DNA was digested with restriction enzymes to recognize 2 sites within the parental vector sequence (AfeI, lane A) or with an enzyme that recognizes a single site in the vector and a single site in the putative integration site (XbaI, lane B). Digested and undigested DNA (lane C) as well as parental Flip-IN cell DNA (lane D) were separated by electrophoresis and hybridized with a radioactive DNA probe.

FIG. 29 is a set of schematic representations of vTK795, vTK796, and vTK797. The abbreviations used in the Figure include: CMV—cytomegalovirus promoter; R—the repeat sequence at the 5′ and 3′ ends of the viral/vector full-length RNA; U5—U5 LTR; ψ—packaging signal; Env—HIV-1 envelope; RRE—HIV-1 Rev response element; cPPT—central polypurine tract; hAAT—human a-1 antitrypsin promoter; β-Gal—β-Galactosidase; WP—woodchuck hepatitis virus posttranscriptional regulatory element; Δu3—self-inactivating deletion in U3.

FIG. 30 is a set of photographs showing in vivo luciferase expression by intraperitoneal delivery of HIV-1 vector TK464 into BalbC mice. The vector expresses the firefly luciferase gene under control of a CMV promoter.

FIG. 31 is a photomicrograph showing 200× magnification of ipilateral cerebral cortex, indicating GFP expression through direct injection of a lentiviral vector expressing GFP into the cerebral cortex of a normal 16 week old cat that was necropsied 8 days post injection.

FIG. 32 is a series of schematic representations of vectors vTK757, vTK759, UNC6.WChFIX, UNC6.WAhFIX, UNC6.EHChFIX, and UNC6.EHahFIX. Abbreviations used in the Figure include: CMV—cytomegalovirus promoter; R—the repeat sequence at the 5′ and 3′ ends of the viral/vector full-length RNA; U5—U5 LTR; ψ—packaging signal; Env—HIV-1 envelope; RRE—HIV-1 Rev response element; cPPT—central polypurine tract; hAAT—human a-1 antitrypsin promoter; hFIX—human factor IX; WP—woodchuck hepatitis virus posttranscriptional regulatory element; Δu3—self-inactivating deletion in U3.

### DETAILED DESCRIPTION

The ability of retroviral vectors to deliver large genetic payloads into nondividing cells opens promising avenues for the introduction of one or more particular nucleic acid sequences into a cell. However, realizing the full potential of the retroviral vector system into a valid delivery modality is currently impeded by the potential for retroviral vector-induced insertional mutagenesis. Accordingly, the presently disclosed subject matter provides cross-packaged retroviral vectors, and methods of making and using the same, that address the risks associated with retroviral vector-mediated insertional mutagenesis, among other problems in the art.

I. Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the presently disclosed subject matter belongs. Although any methods and materials similar or equivalent to those described herein can be used in the practice or testing of the presently disclosed subject matter, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

The term “about”, as used herein when referring to a measurable value such as an amount of weight, time, dose, etc. is meant to encompass in one example variations of ±20% or ±10%, in another example ±5%, in another example ±1%, and in yet another example ±0.1% from the specified amount, as such variations are appropriate to perform the disclosed method.

As used herein, the term “polynucleotide” refers to all forms of DNA and RNA, whether single-stranded, double-stranded, or higher order. A polynucleotide can be chemically synthesized or can be isolated from a host cell or organism. A particular polynucleotide can contain both naturally occurring residues as well as synthetic residues.

The term “therapeutically effective amount” as used herein refers to an amount that results in an improvement or remediation of the symptoms of the disease or condition. More particularly, the term “therapeutically effective amount” as used herein can refer to the amount of a pharmacological or therapeutic agent that will elicit a biological or medical response of a tissue, system, animal or mammal that is being sought by the administrator (such as a researcher, doctor or veterinarian) that includes alleviation of the symptoms of the condition or disease being treated and the prevention, slowing or halting of progression of one or more conditions.

As used herein, the term “vaccine” means a composition comprising an immunogen which, upon administration to an individual, stimulates an immune response. In particular, a vaccine can be an antigenic preparation used to produce active immunity to a disease, in order to prevent or ameliorate the effects of infection by any natural or “wild” strain of the organism. The term “vaccine” also includes any preparation or suspension, including but not limited to a preparation or suspension containing an attenuated or inactive microorganism or subunit thereof or toxin, developed or administered to produce or enhance the body's immune response to a disease or diseases.

A “vector” is a composition that can transduce, transform or infect a cell, thereby causing the cell to express nucleic acids and/or proteins other than those native to the cell, or in a manner not native to the cell. A vector includes a nucleic acid (which in some cases can be RNA or DNA) to be expressed by the cell (a “vector nucleic acid”). A vector can optionally include materials to aid in achieving entry of the nucleic acid into the cell, such as a viral particle, liposome, protein coating or the like. A “cell transformation vector” is a vector which encodes a nucleic acid capable of transforming a cell once the nucleic acid is transduced into the cell.

A “packaging vector” is a vector that encodes components necessary for production of viral particles by a cell transduced by the packaging vector. The packaging vector optionally includes all of the components necessary for production of viral particles, or optionally includes a subset of the components necessary for viral packaging. For instance, in some embodiments, a packaging cell is transformed with more than one packaging vector, each of which has a complementary role in the production of a viral particle.

The term “heterologous” when used with reference to a nucleic acid indicates that the nucleic acid comprises one or more subsequences that are not found in the same relationship to each other in nature. For instance, the nucleic acid can be recombinantly produced, having two or more sequences from unrelated genes arranged to make a new functional nucleic acid. For example, in some embodiments, the nucleic acid has a promoter from one gene arranged to direct the expression of a coding sequence from a different gene. Thus, with reference to the coding sequence, the promoter is heterologous. The term “heterologous” can also be used to refer to a nucleic acid that is not native to a host cell.

As used herein, the term “construct” can be used in reference to nucleic acid molecules that transfer DNA segment(s), RNA segment(s), or combinations thereof from one cell to another. The term “vector” can be used interchangeably with “construct”. The term “construct” can include circular nucleic acid constructs including, but not limited to, plasmid constructs, phagemid constructs, cosmid vectors, as well as linear nucleic acid constructs including, but not limited to, PCR products. The nucleic acid construct can comprise expression signals such as a promoter and/or an enhancer in operable linkage, and can be generally referred to as an “expression vector” or “expression construct”.

Transcriptional and translational control sequences are DNA regulatory sequences, including but not limited to promoters, enhancers, polyadenylation signals, terminators, and the like, that provide for the expression of a coding sequence in a host cell. A “cis-element” can be a nucleotide sequence, also termed a “consensus sequence” or “motif,” that interacts with proteins that can upregulate or downregulate expression of a specific gene locus. A “signal sequence” can also be included with the coding sequence. This sequence encodes a signal peptide, N-terminal to the polypeptide that communicates to the host cell and directs the polypeptide to the appropriate cellular location. Signal sequences can be found associated with a variety of proteins native to prokaryotes and eukaryotes.

“Expression” refers to the transcription of a gene to produce the corresponding mRNA and translation of the mRNA to produce the corresponding gene product, i.e., a peptide, polypeptide, or protein. Gene expression is controlled or modulated by regulatory elements including, but not limited to, 5′ regulatory elements such as promoters.

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “recombinant” when used with reference to a cell indicates that the cell replicates or expresses a nucleic acid, or expresses a peptide or protein encoded by nucleic acid whose origin is exogenous to the cell. Recombinant cells can express genes that are not found within the native (non-recombinant) form of the cell. Recombinant cells can also express genes found in the native form of the cell wherein the genes are re-introduced into the cell by artificial means.

A “recombinant expression cassette” or simply an “expression cassette” is a nucleic acid construct, generated recombinantly or synthetically, with nucleic acid elements which permit transcription of a particular nucleic acid in a cell. The recombinant expression cassette can be part of a plasmid, virus, or other vector. Typically, the recombinant expression cassette includes a nucleic acid to be transcribed, a promoter, and/or other regulatory sequences. In some embodiments, the expression cassette also includes, e.g., an origin of replication, and/or chromosome integration elements (e.g., a retroviral LTR).

A virus or vector “transduces” a cell when it transfers a nucleic acid into the cell. A cell is “transformed” by a nucleic acid when a nucleic acid transduced into the cell becomes stably replicated by the cell, either by incorporation of the nucleic acid into the cellular genome, or by episomal replication. A virus or vector is “infective” when it transduces a cell, replicates, and (without the benefit of any complementary virus or vector) spreads progeny vectors or viruses of the same type as the original transducing virus or vector to other cells in an organism or cell culture, wherein the progeny vectors or viruses have the same ability to reproduce and spread throughout the organism or cell culture. Thus, for example, a nucleic acid construct encoding a retroviral particle is not infective if the nucleic acid construct cannot be packaged by the retroviral particle (e.g., if the nucleic acid lacks a retroviral packaging site), even though the nucleic acid can be used to transfect and transform a cell. Similarly, a retroviral-packageable nucleic acid packaged by a retroviral particle is not infective if it does not encode the retroviral particle that it is packaged in, even though it can be used to transform and transfect a cell. If retroviral-packageable nucleic acid is used to transform a cell infected with a retrovirus in a cell culture or organism infected with a retrovirus, the retroviral-packageable nucleic acid will be replicated and disseminated throughout the organism in concert with the infecting retrovirus. However, the retroviral-packageable nucleic acid is not itself “infective”, because packaging functions are supplied by the infecting retrovirus via trans complementation.

The phrase “retroviral packaging cell line” refers to a cell line (typically a mammalian cell line) that contains the necessary coding sequences to produce viral particles which lack the ability to package RNA and produce replication-competent helper-virus. When the packaging function is provided within the cell line (e.g., in trans), the packaging cell line produces recombinant retrovirus, thereby becoming a “retroviral producer cell line.”

A “retrovirus” is a single stranded, diploid RNA virus that replicates via reverse transcriptase and a retroviral virion. A retrovirus can be replication-competent or replication incompetent. The term “retrovirus” refers to any known retrovirus (e.g., type c retroviruses, such as Moloney murine leukemia virus (MoMuLV), Harvey murine sarcoma virus (HaMuSV), murine mammary tumor virus (MuMTV), gibbon ape leukemia virus (GaLV), feline leukemia virus (FLV) and Rous Sarcoma Virus (RSV). “Retroviruses” of the presently disclosed subject matter also include human T cell leukemia viruses, HTLV-1 and HTLV-2, and the lentiviral family of retroviruses, such as, but not limited to, human immunodeficiency viruses HIV-1 and HIV-2, simian immunodeficiency virus (SIV), feline immunodeficiency virus (FIV), and equine immunodeficiency virus (EIV).

The terms “gag polyprotein”, “pol polyprotein”, and “env polyprotein” refer to the multiple proteins encoded by retroviral gag, pol and env genes which are typically expressed as a single precursor “polyprotein”. For example, HIV gag encodes, among other proteins, p17, p24, p9 and p6. HIV pol encodes, among other proteins, protease (PR), reverse transcriptase (RT) and integrase (IN). HIV env encodes, among other proteins, Vpu, gp120 and gp41. As used herein, the term “polyprotein” shall include all or any portion of gag, pol and env polyproteins.

The terms “Vpx” and “Vpr” refer respectively to lentiviral Vpx and Vpr proteins described, for example, in WO 96/07741, hereby incorporated by reference in its entirety. These terms can also refer to fragments, mutants, homologs and variants of Vpr and Vpx which retain the ability to associate with p6.

The term “transgene” means a nucleic acid sequence (e.g., a therapeutic gene), which is partly or entirely heterologous, i.e., foreign, to a cell into which it is introduced, or, is homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). A transgene can include one or more transcriptional regulatory sequences and any other nucleic acid, such as introns, that may be necessary for optimal expression of a selected nucleic acid.

An “LTR” is a long terminal repeat. LTRs are sequences found in retroviruses. The LTR sequence is typically at least several hundred bases long, usually bearing inverted repeats at its termini (often starting with TGAA and ending with TTCA), and flanked with short direct repeats duplicated within the cell DNA sequences flanking an insertion site. The short inverted repeats are involved in integrating the full length viral, retrotransposon, or vector DNA into the host genome. The integration sequence is sometimes called att, for attachment. Inside the LTRs reside three distinct subregions: U3 (the enhancer and promoter region, transcribed from the 5′-LTR), R (repeated at both ends of the RNA), and U5 (transcribed from the 5′-LTR). The LTR and its associated flanking sequences (primer binding sites, splice sites, dimerization linkage and encapsidation sequences) comprise the cis-acting sequences of a retroviral vector. Sources of LTR nucleic acid sequences, i.e., nucleic acid fragments or segments, include, but are not limited to murine retroviruses, murine VL30 sequences, retrotransposons, simian retroviruses, avian retroviruses, feline retroviruses, lentiviruses, avian retroviruses and bovine retroviruses.

“Isolated”, as used herein, means that a naturally occurring nucleic acid sequence, DNA fragment, DNA molecule, coding sequence, or oligonucleotide is removed from its natural environment, or is a synthetic molecule or cloned product. Preferably, the nucleic acid sequence, DNA fragment, DNA molecule, coding sequence, or oligonucleotide is purified, i.e., essentially free from any other nucleic acid sequence, DNA fragment, DNA molecule, coding sequence, or oligonucleotide and associated cellular products or other impurities.

Several terms herein can be used interchangeably. Thus, “virion”, “virus”, “viral particle”, “viral vector”, “viral construct, and “vector particle” can refer to virus and virus-like particles that are capable of introducing nucleic acid into a cell through a viral-like entry mechanism. Such vector particles can, under certain circumstances, mediate the transfer of genes into the cells they infect. Such cells are designated herein as “target cells”. When the vector particles are used to transfer genes into cells which they infect, such vector particles are also designated “gene delivery vehicles” or “delivery vehicles”. Retroviral vectors have been used to transfer genes efficiently by exploiting the viral infectious process. Foreign genes cloned into the retroviral genome can be delivered efficiently to cells susceptible to infection by the retrovirus. Through other genetic manipulations, the replicative capacity of the retroviral genome can be destroyed. The vectors introduce new genetic material into a cell but are unable to replicate.

The term “envelope protein” as used herein refers to a polypeptide that can be incorporated into an envelope of a retrovirus and can bind target cells and facilitate infection of the target cell by the RNA virus that it envelops. “Envelope protein” is meant to include naturally-occurring (i.e., native) envelope proteins and functional derivatives thereof that can form pseudotyped retroviral virions and exhibit a desired functional characteristics (e.g, facilitate viral infection of a desired target cell, and/or exhibit a different or additional biological activity). In general, envelope proteins of interest in the presently disclosed subject matter include any viral envelope protein that can, in combination with a retroviral genome, retroviral Pol, retroviral Gag, and other essential retroviral components, form a retroviral particle. Such envelope proteins include retroviral envelope proteins derived from any suitable retrovirus (e.g., an amphotropic, xenotropic, ecotropic or polytropic retrovirus) as well as non-retroviral envelope proteins that can form pseudotyped retroviral virions (e.g., VSV G).

With respect to the methods of the presently disclosed subject matter, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the presently disclosed subject matter indicate effectiveness with respect to all vertebrate species which are included in the term “subject.” In this context, a vertebrate is understood to be any vertebrate species in which treatment of a disorder is desirable. As used herein “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like.

II. Chimeric Retroviral Vectors

The presently disclosed subject matter includes chimeric retroviral vectors. In some embodiments, the vectors are constructed to carry or express a selected nucleic acid molecule of interest. The chimeric retrovirus can be used for the in vivo and ex vivo transfer and expression of nucleic acid sequences, among other applications.

There are many retroviruses suitable for use with the presently disclosed subject matter including, but not limited to, murine leukemia virus (MLV), human immunodeficiency virus (HIV), equine infectious anemia virus (EIAV), mouse mammary tumor virus (MMTV), Rous sarcoma virus (RSV), Fujinarni sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-27 (MC27), and Avian erythroblastosis virus (AEV). Chimeric retroviral vectors of the presently disclosed subject matter can be readily constructed from a wide variety of retroviruses, including for example, lentiviruses. Lentiviruses for use in the preparation or construction of chimeric retroviral vectors of the presently disclosed subject matter can include retroviruses such as, but not limited to, Avian Leukosis Virus, Bovine Leukemia Virus, Murine Leukemia Virus, Mink-Cell Focus-Inducing Virus, Murine Sarcoma Virus, Reticuloendotheliosis virus and Rous Sarcoma Virus. Such retroviruses can be readily obtained from depositories or collections such as the American Type Culture Collection (“ATCC”; Rockville, Md., United States of America), or isolated from known sources using techniques available in the art.

In some embodiments of the presently disclosed subject matter, self-inactivating (SIN) vectors can be made by deleting promoter and enhancer elements in the U3 region of the 3′ LTR, including the TATA box and binding sites for one or more transcription factors. The deletion can be transferred to the 5′ LTR after reverse transcription and integration in transduced cells, resulting in the transcriptional inactivation of the LTR in the provirus. Possible advantages of SIN vectors can include increased safety of the gene delivery system as well as the potential to reduce promoter interference between the LTR and the internal promoter, resulting in increased expression of the gene of interest.

In some embodiments, the vectors of the presently disclosed subject matter contain the minimum retroviral sequences necessary direct the desired retroviral function (e.g., packaging of RNA). That is, the remainder of the vector is preferably of non-viral origin, or from a virus other than the first, starting retrovirus. In some embodiments, a first retroviral vector is modified by incorporating a cis element from a second retrovirus into the first retroviral vector, creating a chimeric vector. For example, an HIV-1 cis element can be cloned into a target non-HIV vector to generate a chimeric vector. The HIV-1 cis element can include, but is not limited to, a HIV-1 RRE, an Env gene fragment from the region flanking HIV-1 RRE, HIV-1 cPPT, and combinations thereof. In some embodiments, the chimeric retroviral vector can further comprise a 5′ and/or 3′ long terminal repeat from the non-HIV-1 retrovirus.

In some embodiments, efficient Rev/RRE-dependent cross packaging of a first retroviral vector by a second retroviral packaging system can be achieved. For example, cloning a second retroviral cis element (including but not limited to RRE, a portion of the Env gene that flanks the RRE, and/or cPPT) into the first retroviral vector results in chimeric vectors that can be cross-packaged by the second retroviral packaging cassette. In some embodiments, the inclusion of the second retroviral cis element into the first retrovirus allows the chimeric vector to be packaged with the second retroviral packaging machinery into a second retroviral vector particle. In some embodiments the chimeric vectors retain the capacity to be packaged with the first retroviral packaging system.

The vectors of presently disclosed subject matter can include one or more promoters. Suitable promoters which can be employed include, but are not limited to, the retroviral LTR; the SV40 promoter; and the human cytomegalovirus (CMV) promoter, or any other promoter (e.g., cellular promoters such as eukaryotic cellular promoters including, but not limited to, the histone, pol III, MuLV, SV40, Rous Sarcoma Virus (RSV), vaccinia P7.5, rat β-actin promoters and B-actin promoters). The selection of a suitable promoter will be apparent to those skilled in the art from the teachings contained herein.

The promoter preferably controls the expression of a desired DNA sequence encoding a protein, but can also be operably linked to one or more other genes of interest, e.g. transgenes. The complete enhancer-promoter can be derived as would be apparent to one of skill in the art, or obtained from commercial sources, such as Clontech (Palo Alto, Calif., United States of America), Invitrogen (Carlsbad, Calif., United States of America) and Strategene (Cedar Creek, Tex., United States of America).

In some embodiments, the presently disclosed subject matter employs an inducible promoter within the retroviral vectors, so that transcription of selected genes can be turned on and off. This minimizes cellular toxicity caused by expression of cytotoxic viral proteins, increasing the stability of the packaging cells containing the vectors. For example, high levels of expression of VSV-G (envelope protein) and Vpr can be cytotoxic (Yee, J. K., et al., (1994) Proc. Natl. Acad. Sci., 91:9654-9568) and, thus, expression of these proteins in packaging cells of the presently disclosed subject matter can be controlled by an inducible operator system, such as the inducible Tet operator system (GIBCOBRL, Carlsbad, Calif., United States of America), allowing for tight regulation of gene expression (i.e., generation of retroviral particles) by the concentration of tetracycline in the culture medium. For example, with the Tet operator system, in the presence of tetracycline, the tetracycline is bound to the Tet transactivator fusion protein (tTA), preventing binding of tTA to the Tet operator sequences and allowing expression of the gene under control of the Tet operator sequences (Gossen et al. (1992) PNAS 89:5547-5551). In the absence of tetracycline, the tTA binds to the Tet operator sequences preventing expression of the gene under control of the Tet operator.

Suitable regulatory sequences required for gene transcription, translation, processing and/or secretion are art-recognized, and are selected to direct expression of the desired protein in an appropriate cell. Accordingly, regulatory sequences that can be used in the presently disclosed retroviral vectors include any genetic element present 5′ (upstream) or 3′ (downstream) of the region of a gene and which can control or affect expression of the gene, such as enhancer and promoter sequences. Such regulatory sequences are discussed, for example, in Goeddel, Gene expression Technology: Methods in Enzymology, page 185, Academic Press, San Diego, Calif. (1990), and can be selected by those of ordinary skill in the upon a review of the presently disclosed subject matter.

III. Preparation of Stable Packaging Cell Lines

Stable packaging cell lines can be made by stably transforming a cell (such as but not limited to a mammalian cell) with a packaging vector. Host cells are competent or rendered competent for transformation by various known approaches. There are several well-known methods of introducing DNA into animal cells, including but not limited to calcium phosphate precipitation, fusion of the recipient cells with bacterial protoplasts containing the DNA, treatment of the recipient cells with liposomes containing the DNA, DEAE dextran, receptor-mediated endocytosis, electroporation and micro-injection of the DNA directly into the cells.

The packaging cell lines disclosed herein can be useful for providing the gene products necessary to encapsidate and provide a membrane protein for a retrovirus and retroviral vector. When retroviral sequences are introduced into the packaging cell lines, such sequences are encapsidated with the nucleocapsid proteins and these units then bud through the cell membrane to become surrounded in cell membrane and to contain the envelope protein produced in the packaging cell line. These infectious retroviruses can be used as infectious units per se and/or as gene delivery vehicles.

Packaging cells of the presently disclosed subject matter can comprise one or more separate retroviral vectors that respectively encode all or portions of gag, pol and env and/or 5′ LTR from a first retrovirus, a sequence encoding a second retroviral RRE, and a 3′ LTR from the first retrovirus. Protocols for producing recombinant retroviral vectors, and for transforming packaging cell lines, are well known in the art. Moreover, suitable retroviral sequences that can be used in the presently disclosed subject matter can be obtained from commercially available sources. For example, such sequences can be purchased in the form of retroviral plasmids. Suitable packaging sequences that can be employed in the vectors of the presently disclosed subject matter are also commercially available. Thus, while the presently disclosed subject matter can be described with respect to particular embodiments (e.g., particular lentiviral vectors), other retroviral vectors for use in the presently disclosed subject matter can be prepared in accordance with the guidelines described herein.

In a particular embodiment, the presently disclosed subject matter provides a packaging cell comprising one or more recombinant retroviral vectors. These vectors can be prepared by inserting selected retroviral sequences into a suitable vector (e.g., a commercially available expression plasmid) containing appropriate regulatory elements (e.g., a promoter and enhancer, restriction sites for cloning, marker genes, etc.). This can be achieved using standard cloning techniques, including PCR, as is well known in the art. Retroviral sequences to be cloned into such vectors can be obtained from any known source, including, but not limited to, lentiviral genomic RNA, or cDNAs corresponding to viral RNA. Suitable sources of retroviral cDNA clones include the American Type Culture Collection (ATCC), Rockville, Md., United States of America.

Once cloned into an appropriate vector (e.g., expression vector), retroviral sequences (e.g., gag, pol, env, LTRs and cis-acting sequences) can be modified as described herein. In some embodiments, retroviral sequences amplified from a source such as a plasmid are cloned into a suitable vector, and modified by deletion (using restriction enzymes), substitution (e.g., using site directed mutagenesis), or other (e.g., chemical) modification to prevent expression or function of selected viral sequences. As described in the Examples provided herein, portions of the gag, pol and env genes can be removed or mutated, along with selected accessory genes.

Each vector of the presently disclosed subject matter can contain the minimum retroviral sequences necessary to encode the desired retroviral proteins (e.g., gag, pol and env) or direct the desired retroviral function (e.g., packaging of RNA). That is, the remainder of the vector can be of non-viral origin, or from a virus other than a retrovirus, e.g., HIV. In some embodiments, LTRs contained in the retroviral vectors of the presently disclosed subject matter can be modified by replacing a portion of the LTR with a functionally similar sequence from another virus, creating a chimeric LTR. For example, the lentiviral 5′ LTR, which serves as a promoter, can be partially replaced by the CMV promoter or an LTR from a different retrovirus (e.g., MuLV or MuSV). Alternatively, or additionally, the retroviral 3′ LTR can be partially replaced by a polyadenylation sequence. By minimizing the total retroviral sequences within the vectors of the presently disclosed subject matter in this manner, the chance of recombination among the vectors, leading to replication-competent retrovirus, is greatly reduced.

Any suitable expression vector can be employed in the presently disclosed subject matter. As described in Examples below, suitable expression constructs include a human cytomegalovirus (CMV) immediate early promoter construct. The cytomegalovirus promoter can be obtained from any suitable source. For example, the complete cytomegalovirus enhancer-promoter can be derived from the human cytomegalovirus (hCMV). Other suitable sources for obtaining CMV promoters include commercial sources, such as Clontech (Mountain View, Calif., United States of America), Invitrogen (Carlsbad, Calif., United States of America) and Stratagene (La Jolla, Calif., United States of America).

Suitable regulatory sequences required for gene transcription, translation, processing and secretion are art-recognized, and can be selected to direct expression of the desired protein in an appropriate cell. Accordingly, the term “regulatory sequence”, as used herein, includes any genetic element present 5′ (upstream) or 3′ (downstream) of the translated region of a gene and which control or affect expression of the gene, such as enhancer and promoter sequences. Such regulatory sequences are discussed, for example, in Goeddel, Gene expression Technology: Methods in Enzymology, page 185, Academic Press, San Diego, Calif. (1990), and can be selected by those of ordinary skill in the art for use in the present presently disclosed subject matter.

In addition to encoding the necessary retroviral proteins for production and assembly of core virions (e.g., gag and pol proteins), packaging cell lines of the presently disclosed subject matter can also encode viral envelope proteins (env) that determine the range of host cells which can ultimately be infected and transformed by recombinant retroviruses generated from the cell lines. In some embodiments, the viral env proteins expressed by packaging cells of the presently disclosed subject matter are encoded on a separate vector from the viral gag and pol genes.

Examples of retroviral-derived env genes that can be employed in the presently disclosed subject matter include, but are not limited to type C retroviral envelope proteins, such as those from Moloney murine leukemia virus (MoMuLV), Harvey murine sarcoma virus (HaMuSV), murine mammary tumor virus (MuMTV), gibbon ape leukemia virus (GaLV), and Rous Sarcoma Virus (RSV). Other viral env genes which can be used include, for example, env genes from immunodeficiency viruses (HIV-1, HIV-2, FIV, SIV and EIAV), human T cell leukemia viruses (HTLV-1 and HTLV-3), and Vesicular stomatitis virus (VSV) (Protein G). When producing recombinant retroviruses of the presently disclosed subject matter (e.g., chimeric retroviruses), the wild-type retroviral env gene can be used, or can be substituted with any other viral env gene, such those listed above. Methods of pseudotyping recombinant viruses with envelope proteins from other viruses in this manner are known in the art. As referred to herein, a “pseudotype envelope” is an envelope protein other than the one that naturally occurs with the retroviral core virion, which encapsidates the retroviral core virion (resulting in a phenotypically mixed virus).

Viral envelope proteins of the presently disclosed subject matter (whether pseudotyped or not) can also be modified, for example, by amino acid insertions, deletions or mutations to produce targeted envelope sequences, synthetic and/or other hybrid envelopes; derivatives of the VSV-G glycoprotein. Furthermore, it is possible to limit the infection spectrum of retroviruses and consequently of retroviral-based vectors, by modifying the viral packaging proteins on the surface of the viral particle. For instance, strategies for the modification of the infection spectrum of retroviral vectors include: coupling antibodies specific for cell surface antigens to the viral env protein or coupling cell surface receptor ligands to the viral env proteins. Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector in to an amphotropic vector.

In one embodiment, the presently disclosed subject matter provides packaging cells that produce recombinant retrovirus (e.g., HIV, SIV, FIV, EIV) pseudotyped with the VSV-G glycoprotein. The VSV-G glycoprotein has a broad host range. Therefore, VSV-G pseudotyped retroviruses demonstrate a broad host range (pantropic) and are able to efficiently infect cells that are resistant to infection by ecotropic and amphotropic retroviruses. Any suitable serotype and strain (e.g., VSV Indiana, San Juan) of VSV-G can be used in the presently disclosed subject matter. The protein selected to pseudotype the core virion determines the host range of the packaging cell line. VSV-G interacts with a specific phospholipid on the surface of mammalian veils. Thus, packaging cell lines that utilize VSV-G to provide a pseudotyped envelope for the retroviral core virion can have a broad host range. Moreover, VSV-G pseudotyped retroviral particles can be concentrated more than 100-fold by ultracentrifugation. Stable VSV-G pseudotyped retrovirus packaging cell lines permit generation of large scale viral preparations (e.g. from 10 to 50 liters supernatant) to yield retroviral stocks in the range of 10⁷ to 10¹¹ retroviral particles per mL.

The culture of cells used in conjunction with the presently disclosed subject matter, including cell lines and cultured cells from tissue or blood samples, can be accomplished using techniques disclosed herein and known in the art. Freshney (Culture of Animal Cells, a Manual of Basic Technique, third edition Wiley-Liss, New York (1994)) and the references cited therein provides a general guide to cell culturing. See, also Kuchler et al. (1977) Biochemical Methods in Cell Culture and Virology, Kuchler, R. J., Dowden, Hutchinson and Ross, Inc. Mammalian cell systems can be in the form of monolayers of cells, although mammalian cell suspensions can also be used. Illustrative examples of mammalian cell lines include VERO and Hela cells, Chinese hamster ovary (CHO) cell lines, W138, BHK, Cos-7 or MDCK cell lines (see, e.g., Freshney, supra).

Supernatants from cell cultures of the packaging cells of the presently disclosed subject matter can be obtained by approaches disclosed herein and using standard techniques such as those taught in Freshney, supra. See also, Corbeau et al. (1996) Proc. Natl. Acad. Sci. USA 93:14070-14075 and the references therein. Components from the cell supernatants can be further purified using standard techniques. For example, retroviral particles in the supernatant can be purified from the supernatant by methods typically used for viral purification, including but not limited to centrifugation, chromatography, affinity purification procedures, and the like.

Transforming mammalian cells with nucleic acids can involve, for example, incubating competent cells with a construct (e.g., plasmid, viral vector) containing nucleic acids which code for a retroviral particle. The construct that is used to transform the host cell preferably contains nucleic acid sequences to initiate transcription and sequences to control the translation of the encoded sequences. These sequences are referred to generally as expression control sequences. Illustrative mammalian expression control sequences can be obtained from the SV-40 promoter, for example. A cloning vector containing expression control sequences is cleaved using restriction enzymes and adjusted in size as necessary or desirable and ligated with DNA coding for the retroviral sequences of interest by means well known in the art.

Co-constructs can be used in selection methods. In these methods, a construct containing a selectable marker, such as an antibiotic resistance gene, is used to co-transfect a cell in conjunction with a construct encoding retroviral packaging nucleic acids. The cells are selected for antibiotic resistance, and the presence of the construct of interest can be confirmed by Southern analysis, Northern analysis, or PCR. Co-constructs encoding proteins to be expressed on the surface of a retroviral particle (e.g., proteins which expand the host range of the capsid such as the VSV envelope, a cell receptor ligand, or an antibody to a cell receptor) are optionally transduced into the packaging cell. In addition to VSV, the envelope proteins of other lipid enveloped viruses can be incorporated into a particle of the presently disclosed subject matter, thereby expanding the transduction range of the particle.

Any suitable cell line can be employed to prepare packaging cells of the presently disclosed subject matter. In some embodiments, the cells used to produce the packaging cell line are mammalian cells, including but not limited to human cells. Suitable human cell lines which can be used include, for example, 293 cells (Graham et al. (1977) J. Gen. Virol., 36:59-72), tsa 201 cells (Heinzel et al. (1988) J. Virol., 62:3738), and NIH3T3 cells (ATCC, Rockville, Md., United States of America)). Other suitable packaging cell lines for use in the presently disclosed subject matter include, but are not limited to, other human cell line-derived (e.g., embryonic cell line derived) packaging cell lines and murine cell line-derived packaging cell lines, such as Psi-2 cells (Mann et al. (1983) Cell, 33:153-159; FLY (Cossett et al. (1993) Virol., 193:385-395; BOSC 23 cells (Pear et al. (1993) PNAS 90:8392-8396; PA317 cells (Miller et al. (1986) Molec. and Cell. Biol., 6:2895-2702; Kat cell line (Finer et al. (1994) Blood, 83:43-50; GP+E cells and GP+EM12 cells (Markowitz et al. (1988) J. Virol., 62:1120-1124, and Psi Crip and Psi Cre cells (U.S. Pat. No. 5,449,614; Danos, O. and Mulligan et al. (1988) PNAS 85:6460-6464). Packaging cell lines of the presently disclosed subject matter can produce retroviral particles having a pantropic, amphotropic, or ecotropic host range. Packaging cell lines can produce retroviral particles, such as lentiviral particles (e.g., HIV-1, HIV-2 and SIV) capable of infecting dividing, as well as non-dividing cells.

IV. Methods of Making Producer Cell Line

When an effective producer cell has been identified, a stable cell line that carries the vector of the presently disclosed subject matter and expresses the nucleic acid fragment encoding the analog can be produced. The stable cell line secretes or carries the analog of the presently disclosed subject matter, thereby facilitating purification thereof.

The packaging cell can be transfected with the minimal vector construct to make a producer cell. The producer cell can comprise: (i) a gag/pol coding sequence; (ii) a viral envelope coding sequence; and (iii) a chimeric vector construct comprising a cis element. The producer cell can be cultured to produce virions containing the minimal vector of the presently disclosed subject matter. The minimal vector is the RNA version of the minimal vector construct. In some embodiments, the RNA vector further comprises an internal promoter, as described herein, operably linked to the transgene. The virions are used to infect desired target cells, thereby transferring the transgene to the target cell.

Although the cells described herein produce retroviral solutions of titers in the range 5×10⁶ to 2×10⁸ IU/mL in the culture medium, such particles can be concentrated further by standard concentration techniques to achieve titers in the range of 5×10⁹ to 2×10¹¹ IU/mL.

In some embodiments, the concentrating step is ultracentrifugation, filtration, or chromatography. The pellet can also be resuspended in a liquid and subjected to a second cycle of ultracentrifugation.

V. Assaying for HIV Packaging Vectors, Packageable Nucleic Acids and HIV Particles in Packaging Cell Lines, Target Cells and Cell Lysates

A wide variety of formats and labels are available and appropriate for detection of packaging vectors, packageable nucleic acids and viral particles in packaging cells, target cells, subjects and cell lysates. Viral antibodies, and the polypeptides and nucleic acids of the presently disclosed subject matter can be detected and quantified by any of a number of approaches known to those of skill in the art, including but not limited to analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion, immunoelectrophoresis, radioimmunoassays, enzyme-linked immunosorbent assays (ELISAs), immunofluorescent assays, and the like.

Nucleic acids can be detected using known methods, such as Southern analysis, Northern analysis, gel electrophoresis, PCR, radiolabeling and scintillation counting, and affinity chromatography. Many assay formats are appropriate, including those reviewed in Tijssen (1993) Laboratory Techniques in Biochemistry and Molecular Biology—Hybridization with Nucleic Acid Probes Parts I and II, Elsevier, N.Y. and Choo (ed) (1994) Methods In Molecular Biology Volume 33—In Situ Hybridization Protocols Humana Press Inc., New Jersey (see also, other books in the Methods in Molecular Biology series).

A variety of automated solid-phase detection techniques are also appropriate. For instance, very large scale immobilized polymer arrays can be used for the detection of nucleic acids. See, Tijssen (supra), Fodor et al. (1991) Science, 251: 767-777 and Sheldon et al. (1993) Clinical Chemistry 39(4): 718-719. Finally, PCR is also routinely used to detect nucleic acids in biological samples (see, Innis, M. A. and Gelfand. D. H. (1990) Optimization of PCRs. pp. 3-12 in: PCR Protocols (Innis, Gelfand, Sninsky and White, eds.) Academic Press, New York for a general description of PCR techniques).

In some embodiments, antibodies are used to detect proteins expressed by the packaged vector or to monitor circulating viral levels in blood, e.g., to monitor the in vivo effect of a therapeutic agent encoded by the packaged nucleic acids. Methods of producing polyclonal and monoclonal antibodies are known to those of skill in the art, and many anti-viral antibodies are available. See, e.g., Coligan (1991) Current Protocols in Immunology Wiley/Greene, N.Y.; and Harlow and Lane (1989) Antibodies: A Laboratory Manual Cold Spring Harbor Press, N.Y.; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256: 495-497.

Polypeptides encoded by the nucleic acids of the presently disclosed subject matter can be used to make antibodies for the detection of retroviral particles using known techniques. Polypeptides of relatively short size can be synthesized in solution or on a solid support. See, e.g., Merrifield (1963) J. Am. Chem. Soc. 85:2149-2154. Various automatic synthesizers are commercially available and can be used in accordance with known protocols. See, e.g., Stewart and Young (1984) Solid Phase Peptide Synthesis, 2d. ed., Pierce Chemical Co. Larger polypeptides can be synthesized recombinantly in prokaryotes or in eukaryotes. See, See, e.g., Sambrook, supra for details concerning cloning and expressing polypeptides, e.g., in E. coli. Expression systems for expressing polypeptides are available using E. coli., Bacillus sp. (Palva, I. et al., 1983, Gene 22:227-235; Mosbach, K. et al., Nature, 302:543 545) and Salmonella. E. coli. systems are the most common, and best defined prokaryotic expression systems and are, therefore, preferred. Expression in yeast and other eukaryotic cells, including mammalian cells is also well known and appropriate. See, e.g., Sherman et al. (1982) Methods in Yeast Genetics, Cold Spring Harbor Laboratory. See, e.g., Goeddel, supra; Krieger, Gene Transfer and Expression—A Laboratory Manual, Stockton Press, New York, N.Y., (1990) and the references cited therein; and, Scopes (1982) Protein Purification: Principles and Practice Springer-Verlag New York.

In some embodiments, polypeptides and their corresponding antibodies can be labeled by joining, either covalently or non covalently, a substance which provides for a detectable signal. A wide variety of labels and conjugation techniques are known and are reported extensively in both the scientific and patent literature. Suitable labels include, but are not limited to, radionucleotides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,325; 4,277,437; 4,275,149; and 4,366,241. Also, recombinant immunoglobulins may be produced. See, U.S. Pat. No. 4,816,567; and Queen et al. (1989) Proc. Nat'l Acad. Sci. USA 86: 10027-10033.

VI. Nucleic Acid Delivery

Novel vectors and packaging cell lines of the presently disclosed subject matter can be used to produce recombinant retroviruses that are capable of transferring (and in some embodiments efficiently integrating) heterologous DNAs (e.g., a therapeutic transgene) into host cells. That is, in some embodiments, the presently disclosed subject matter provides a method for introducing an exogenous polynucleotide into a recipient cell, including in some embodiments into the chromosome of a recipient cell. The method can include contacting a recipient cell with a chimeric retrovirus produced by the disclosed methods. In some embodiments, the chimeric retrovirus is transiently expressed. In some embodiments it is integrated into the chromosome. The chimeric retroviral particles generated in accordance with the presently disclosed subject matter can be used to facilitate delivery of a nucleotide sequence of interest to a host cell either in vivo or in vitro.

For example, the chimeric retroviral vector particles can be used in gene therapy applications to deliver one or more therapeutic gene product-encoding sequence to a subject. The chimeric retroviral particles can also be used to develop various disease or development animal or in vitro models. Recipient cells for delivery of chimeric retroviral particles suitable for use with the presently disclosed subject matter include, but are not limited to, endothelial cells, myeloid cells, bone marrow cells, stem cells, lymphocytes, hepatocytes, fibroblasts, lung cells, muscle cells, embryonic cells, and neuronal cells. Methods of administering retroviral particles to a subject to accomplish in vivo transformation are well known in the art (See, for example, Mulligan (1993) Science 260:926; Anderson (1992) Science 256:808; Miller (1992) Nature 357:455; and Crystal (1995) Science 270:404). Methods for in vitro transformation using retroviral particles are also well known in the art.

Gene therapy thus includes any one or more of the addition, the replacement, the deletion, the supplementation, the manipulation, etc. of one or more nucleotide sequences in, for example, one or more target cells. By way of example, gene therapy provides an approach by which any one or more of a nucleotide sequence, such as a gene, can be applied to replace or supplement a defective gene; a pathogenic gene or gene product can be eliminated; a new gene can be added in order, for example, to create a more favorable phenotype; cells can be manipulated at the molecular level to treat cancer (Schmidt-Wolf and Schmidt-Wolf, 1994, Annals of Hematology 69;273-279) or other conditions, including but not limited to, immune, cardiovascular, neurological, inflammatory or infectious disorders. In some embodiments, antigens can be manipulated and/or introduced to elicit an immune response, such as genetic vaccination.

A variety of genes or DNA fragments can be incorporated into the retroviral vector particles of the presently disclosed subject matter for use in gene therapy. Proteins of use include, but are not limited to, various hormones, growth factors, enzymes, lymphokines, cytokines, receptors, and the like.

Among the genes that can be transferred in accordance with the presently disclosed subject matter are those encoding polypeptides that are absent, are produced in diminished quantities, or are produced in mutant forms in subjects suffering from a genetic disease. Other genes of interest include, but are not limited to, those that encode proteins that have been engineered to circumvent a metabolic defect or proteins that, when expressed by a cell, can adapt the cell to grow under conditions where the unmodified cell would be unable to survive, or would become infected by a pathogen.

In addition to protein-encoding genes, the presently disclosed subject matter can be used to introduce nucleic acid sequences encoding medially useful RNA molecules into cells. Examples of such RNA molecules include, but are not limited to, anti-sense molecules, siRNA and other molecules for RNAi methods, and catalytic molecules, such as ribozymes.

The presently disclosed recombinant retroviruses can be used to transform not only a variety of dividing cell types, but also non-dividing cell types, increasing the range of diseases treatable by gene therapy. For instance, these recombinant retroviruses can be used to transform neuron, muscle, heart, lung, liver, skin, and bone marrow cells.

Novel packaging cell lines of the presently disclosed subject matter can be used to produce recombinant retroviruses (e.g., recombinant lentiviruses), free of unwanted helper-virus, which are capable of transferring (and efficiently integrating) heterologous DNAs (e.g., a therapeutic transgene) into eukaryotic cells. That is, recombinant retrovirus can be harvested from packaging cell lines of the presently disclosed subject matter and used as viral stock to infect recipient cells in culture or in vivo. In the case of secreted proteins or proteins expressed in hematopoietic cells, sensitive assays such as ELISA or Western blotting can be used to assess gene transfer efficiency.

A wide variety of heterologous DNAs can be transferred to cells via the presently disclosed subject matter. Such DNAs include, for example, therapeutic genes (e.g., encoding therapeutic proteins which can be used to treat diseases). Because non-dividing, as well as dividing, cells can be transformed via recombinant retroviruses (e.g., lentiviruses) of the presently disclosed subject matter, treatable diseases include but are not limited to, for example, globin disorders, blood coagulation factor deficiency, neural disorders, autoimmune diseases, lung diseases. Thus, suitable therapeutic genes to be transferred can include, for example, human β-globin, Factor VIII, Factor IX and Cystic Fibrosis genes. Alternatively, retroviral vectors of the presently disclosed subject matter can be used to deliver polynucleotides, such as antisense polynucleotides and siRNA, to cells to inhibit expression of selected genes (Yee, supra; Dranoff, G. et al., Proc. Natl. Acad. Sci., 90:3539-3543 (1993); Miller, A. D., et al., Meth. in Enz., 217:581-599 (1993)).

In addition, the presently disclosed subject matter can also be used to produce retroviruses containing DNA of interest for introducing sequences of interest into mammalian cells, such as human cells, which will subsequently be administered into localized areas of the body (e.g., ex vivo infection of autologous white blood cells for delivery of protein into localized areas the body, see e.g., U.S. Pat. No. 5,399,326).

The vector particles generated from the packaging cell line can be targetable, whereby a receptor binding region enables the vector particles to bind to a target cell. The retroviral vector particles thus can be directly administered to a desired target cell ex vivo, and such cells may then be administered to a subject as part of a gene therapy procedure.

Although the vector particles can be administered directly to a target cell, the vector particles can be engineered such that the vector particles are “injectable” as well as targetable; i.e., the vector particles are resistant to inactivation by the subject's serum, and thus the targetable vector particles can be administered to a subject by intravenous injection, and travel directly to a desired target cell or tissue without being inactivated by the subject's serum. Vector particles generated from such packaging lines, therefore, can be “targetable” and/or “injectable,” whereby such vector particles, upon administration to a subject, travel directly to a desired target cell or tissue.

The targetable vector particles can be useful for the introduction of desired heterologous genes into target cells ex vivo. Such cells can then be administered to a subject as a gene therapy procedure, whereas vector particles which are targetable and injectable may be administered in vivo to the subject, whereby the vector particles travel directly to a desired target cell.

### EXAMPLES

The following Examples have been included to provide illustrations of the presently disclosed subject matter. In light of the present disclosure and the general level of skill in the art, those of skill will appreciate that the following Examples are intended to be exemplary only and that numerous changes, modifications and alterations can be employed without departing from the spirit and scope of the presently disclosed subject matter.

### Example 1

### A Uniform HIV-1 Rev/RRE-Based Packaging System Can Support Efficient Cross-Packaging and Subsequent Stable Transduction of FIV, EIAV, and MLV Vectors

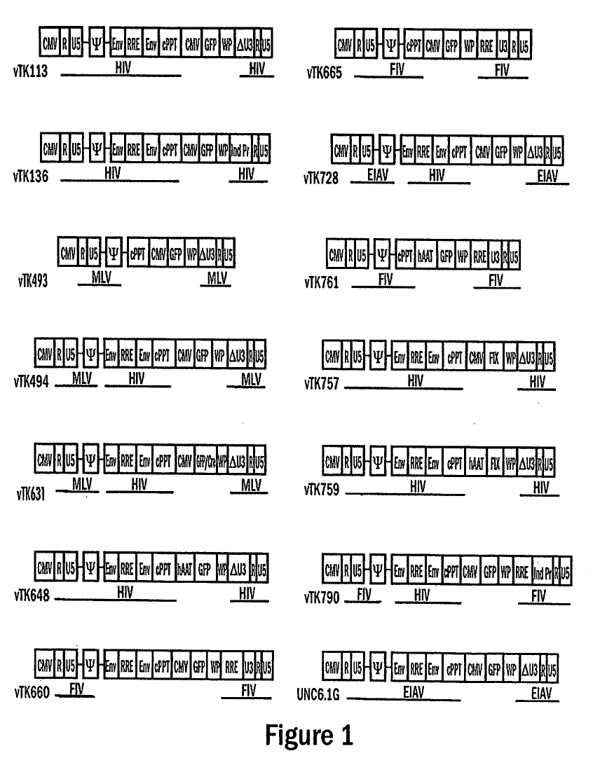

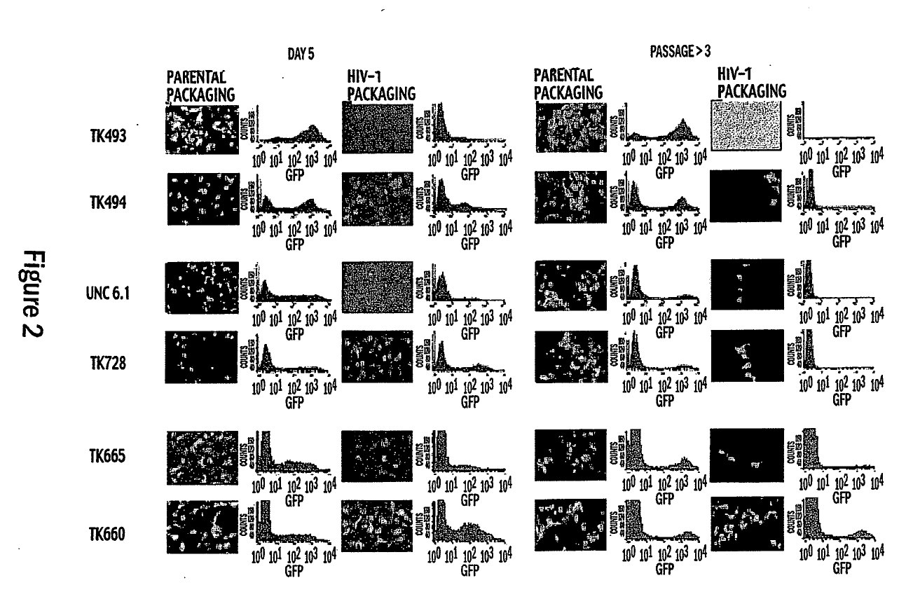

Efficient Rev/RRE dependent cross-packaging of FIV, EIAV, and MLV vectors by the HIV-1 packaging system has been achieved. A 1 kbp sequence containing the HIV-1 RRE (SEQ ID NO: 1), a portion of the Env gene that flanks the RRE (SEQ ID NOs:2 and 3), and HIV-1 cPPT (SEQ ID NO:4) has been identified. Cloning the DNA fragment containing this sequence into FIV, EIAV, and MLV vectors resulted in the generation of chimeric vectors, which could be cross-packaged by the HIV-1 packaging cassette (FIGS. 1 and 2).

The ability to cross-package the chimeric vectors with the HIV-1 packaging system was tested by the 3 plasmid transient transfection method of vector production. Each parental and chimeric vector construct was co-transfected into 293T cells along with VSV-G envelope expression cassette and either the HIV-1 packaging construct ΔNRF (from which HIV-1 gag/pol, tat, rev and vpu genes are expressed under the control of a CMV promoter), or the relevant parental packaging cassette. All vectors contain the GFP marker gene under control of the GFP promoter. Target 293T cells were exposed to conditioned media and efficiency of vector production was determined by scoring GFP expression using either fluorescence microscopy of FACS analysis. The transduced cells were cultured for 2 weeks after which GFP expression was determined by fluorescence microscopy and FACS analysis.

The efficiency of cross-packaging varied between the three chimeric vectors. The FIV and MLV based chimeric vector showed the highest and lowest levels of cross-packaging, respectively. Similarly, the level of transgene (GFP) expression was the highest in FIV chimeric vector-transduced cells. Further, only the FIV chimeric vector maintained long term transgene expression in culture. The rapid decline in transgene expression in MLV and EIAV chimeric-transduced cells was associated with cell passaging. This observation implied that cross-packaged MLV and EIAV chimeric vectors lack the ability to integrate into the host genome, likely due to sequence differences between HIV-1 and MLV and EIAV att sites.

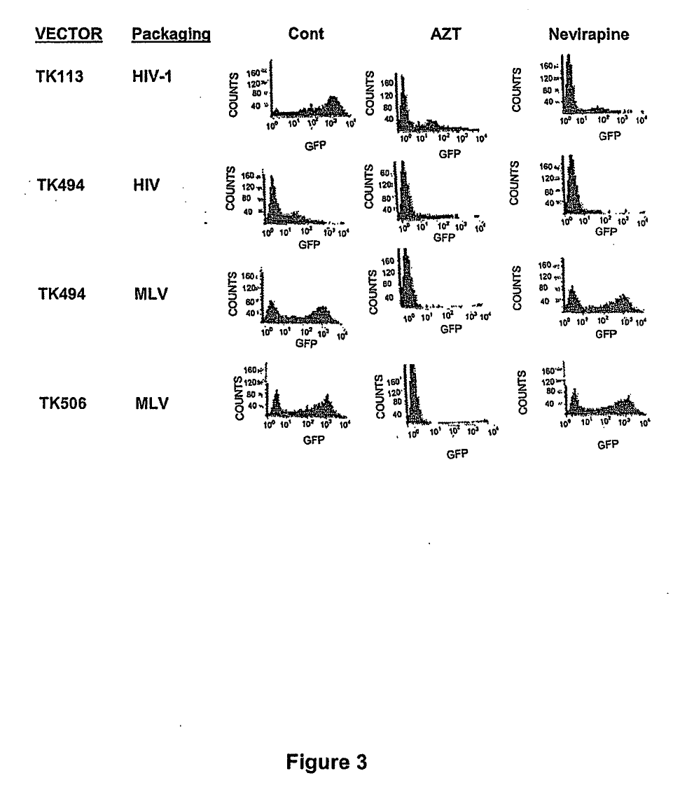

The possibility that the observed cross-packaging is a result of vector pseudo-transduction was ruled out because transduction of 293T cells by traditional MLV vector and cross-packaged chimeric MLV vector was inhibited by AZT (FIG. 3). AZT is a nucleoside analog inhibitor of reverse transcription. Nevirapine, a non-nucleoside inhibitor of HIV-1 reverse transcriptase mediated similar inhibition of MLV chimeric vector transduction of 293T cells. The presence of nevirapine had no effect on the ability of the traditional MLV vector to transduce 293T cells, indicating that the sensitivity of cross-packaged vector to nevirapine is determined by the origin of the packaging construct, not by cis elements in the vector genome. Thus, the ability to compare the properties of a cross-packaged vector with the properties of its parental vectors can be used as a means to dissect the relative effect of the vector cis and trans elements on its basic biologic properties. The results demonstrate that non-HIV-1 vectors can be efficiently cross-packaged by the HIV-1 packaging machinery in a RRE/Rev dependent manner.

### Example 2

### Mechanisms Involved in the Lack of Chimeric EIAV/HIV-1 Vector Integration and High Levels of Transgene Expression

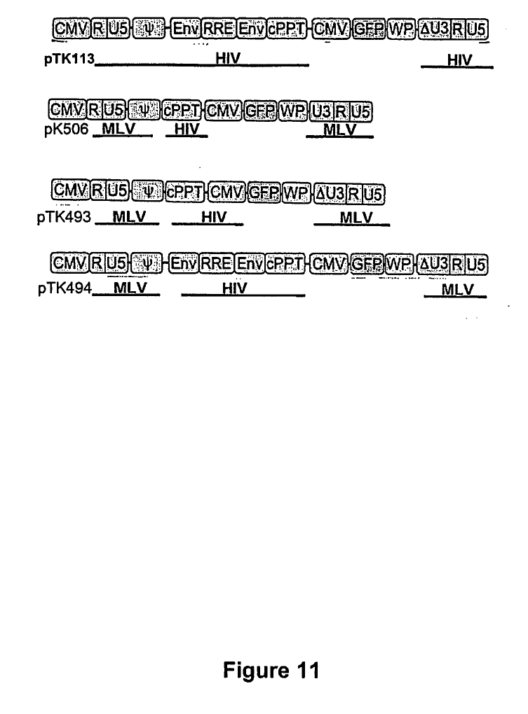

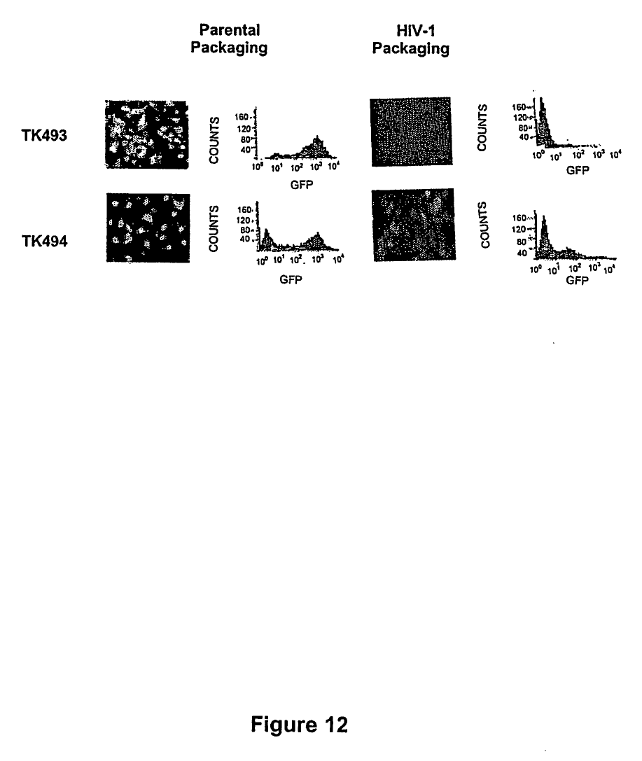

The chimeric MLV/HIV-1 vectors in which DNA sequences including the HIV-1 RRE and cPPT (pTK494, FIG. 11), HIV-1 RRE, cPPT and the HIV-1 5′ untranslated region (pTK497), HIV-1 RRE cPPT, as well as the HIV-1 5′ untranslated region and the primer-binding site (pTK498, FIG. 1) were incorporated upstream to the internal CMV promoter in a traditional MLV-based vector. The incorporation of the HIV-1 sequences did not alter transgene (GFP) expression from the MLV vectors, but a significant reduction in vector titers (3-10 fold) was observed. Unexpectedly, as shown in FIGS. 11 and 12, HIV-1 RRE containing MLV vectors were packaged efficiently into productive HIV-1 particles, thus indicating that the HIV-1 RRE/Rev can serve as a secondary packaging system.

Using the reverse transcription RT inhibitor AZT, it was demonstrated that RT is required for chimeric MLV/HIV-1 vectors (FIG. 3). Further, these results ruled out the possibility that GFP expression in chimeric vector transduced cells was a result of pseudotransduction (FIG. 3).

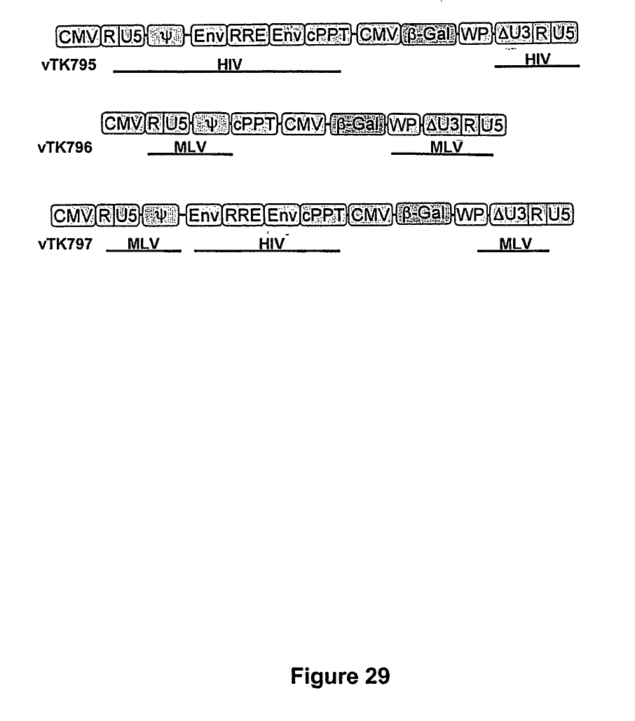

To further characterize this phenomenon, the effects of the parental MLV packaging signal on vector titers were determined. To this end, chimeric MLV/HIV-1 vectors from which either the 5′ end of the MLV packaging signal (pTK802) or the 5′ end and the dimerization domain in the MLV packaging signal (pTK797, FIG. 29) had been deleted were developed.

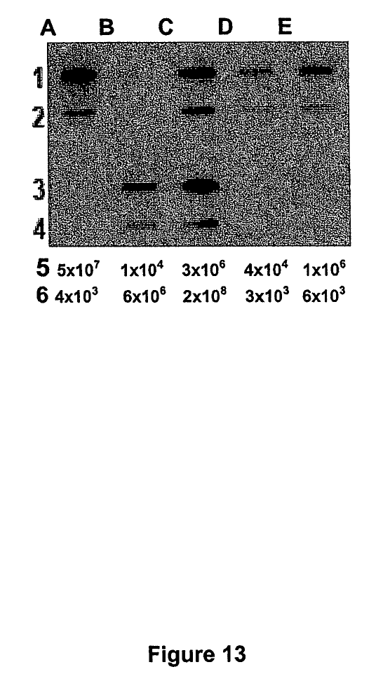

Vector particles were packaged with either the HIV-1 or the MLV packaging system, and titered by scoring GFP expression following serial dilutions on 293T cells. As shown in FIG. 13, the deletions in the packaging signal significantly reduced the titers of the MLV-packaged vectors, while only minor or no effect was observed upon packaging of these vectors with the HIV-1 packaging system.

To characterize the effects of the mutations in the MLV packaging signal on vector-mRNA encapsidation, Northern slot blot analysis using a ³²P labeled probe directed to the CMV promoter on equal amounts of vector particles (normalized RT assay) was used. As shown in FIG. 13, the HIV-1 vector vTK113 and the MLV packaging signal-deleted chimeric vectors vTK797 and vTK802 were not encapsidated efficiently into MLV particles. In contrast, only the RRE-packaging vector vTK493 MLV failed to efficiently encapsidated into HIV-1 particles. These data indicate that the RRE/Rev system is involved in the packaging of chimeric vectors into HIV-1 particles.

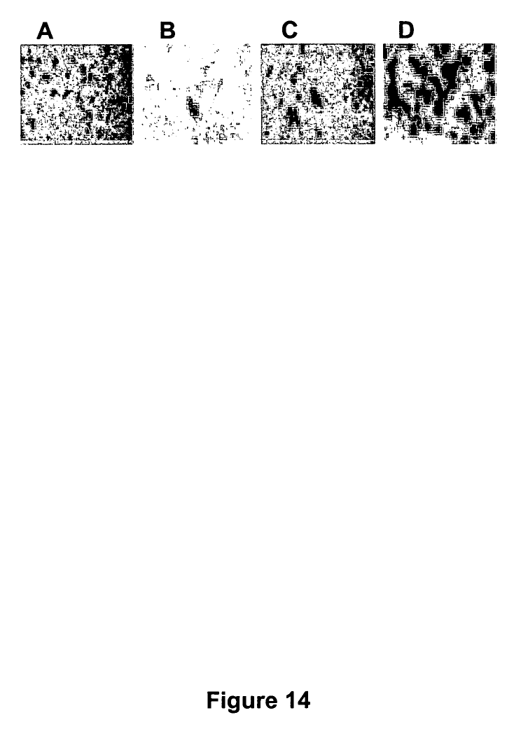

Deleting the parental packaging signal from pTK631, which resulted in the generation of pTK816, did not affect the ability of the HIV-1 packaging system to package and generate productive chimeric vectors (FIG. 14). However, a dramatic decrease in the titers of MLV packaged chimeric vectors (FIG. 14) was observed. These results indicate that the Rev/RRE mediated packaging of MLV vectors is independent of the parental MLV packaging signal.

To determine the characteristics of the chimeric vector, the ability of the HIV-1 and MLV packaged chimeric vectors to transduce aphidicolin-arrested cells was compared. As shown in FIG. 14, in contrast to RRE devoid vectors, chimeric vectors packaged by the HIV-1 packaging system transduced growth arrested cells efficiently. Another HIV-1 feature, which is characteristic of the chimeric vectors, is their sensitivity to nevirapine, an inhibitor of the HIV-1 reverse transcriptase (FIG. 3).

Prompted by the fact that traditional HIV-1 vector packaging cassettes contain the parental HIV-1 RRE sequence, the ability of HIV-1 vector particles to package mRNAs encoding the HIV-1 gag/pol genes was characterized.

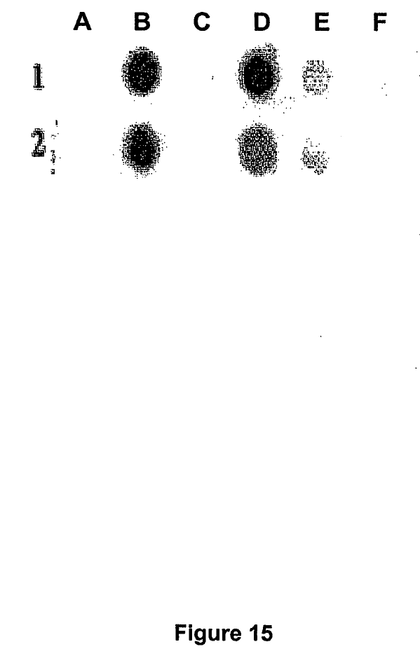

To this end, the HIV-1 vector vTK113, the chimeric vector vTK494 and the MLV based vector vTK493 were packaged by either the HIV-1 or the MLV packaging system. RNA extracted from conditioned media containing the above vector particles was subjected to a dot blot analysis using a ³²P labeled probe directed to the HIV-1 pol gene. As shown in FIG. 15, vector particles packaged by the HIV-1 packaging machinery contained pol-encoding mRNAs.

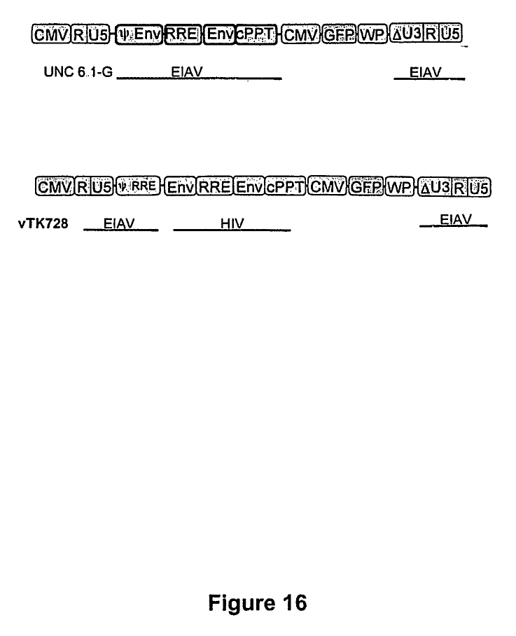

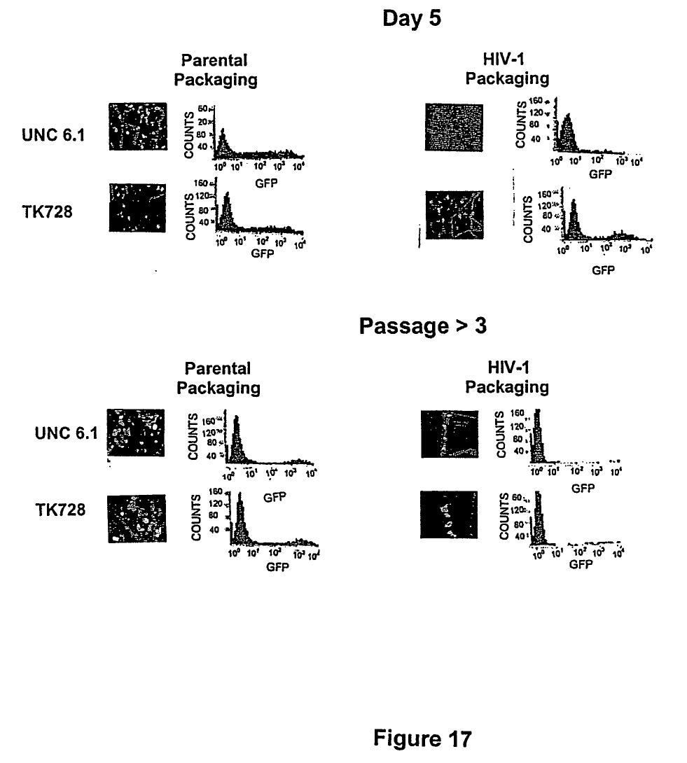

The ability of HIV-1 RRE/Rev system to mediate packaging of EIAV/HIV-1 chimeric vectors into HIV-1 particles was tested. To this end, the HIV-1 RRE was incorporated into the traditional EIAV vector UNC-1 to generate the chimeric vector pTK728 (FIG. 16). As shown in FIG. 17, both the EIAV and HIV-1 packaging system efficiently packaged the chimeric EIAV/HIV-1. Importantly, the titer (2-3×10⁵ IU/mL) and the high level of transgene expression from the HIV-1 packaged chimeric vector were comparable to the titer and level of expression from the EIAV packaged vectors.

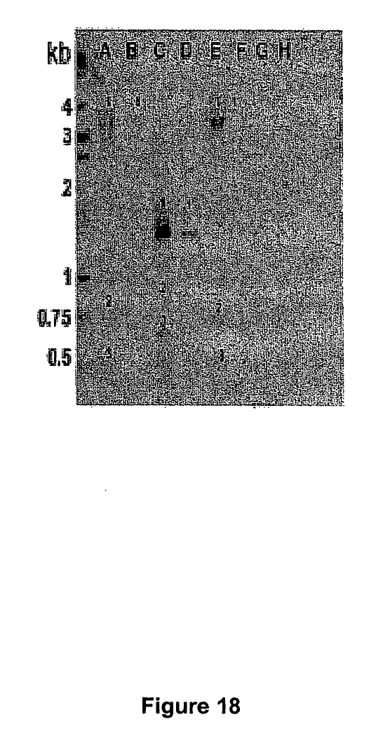

Unexpectedly, 293T cells transduced with the HIV-1 packaged chimeric vector did not maintain transgene (GFP) expression following 2-3 passages in culture (FIG. 17). Using Southern blot analysis (FIG. 18), it was demonstrated that the loss of expression in cells transduced by HIV-1 packaged chimeric vector is due to the loss of the vector genome. The fact that linear and circular episomal vector forms were detected, which could not be detected after 3-5 passages in culture, indicated that the HIV-1 packaged chimeric EIAV/HIV-1 vectors failed to integrate into the host cell's genome.

To facilitate the production of chimeric vectors, a stable producer cell line was established, SODk-1cE/H. The cell line is based on the HIV-1 tetracycline inducible packaging cell line SODk-1, which was described by Kafri, T., et al. (1999) J Virol 73:576-584. To establish the SODk-1cE/H cell line, the vector coding sequences in pTK728 were cloned into pcDNA-Zeo (available from Invitrogen, Carlsbad, Calif., United States of America) to generate pTK799, which was stably transfected into SODk-1 cells by zeocin selection. Twenty (20) single cell clones were isolated and screened for vector production. Currently, the best producer clone yields titers of 2-5×10⁵ IU/mL.

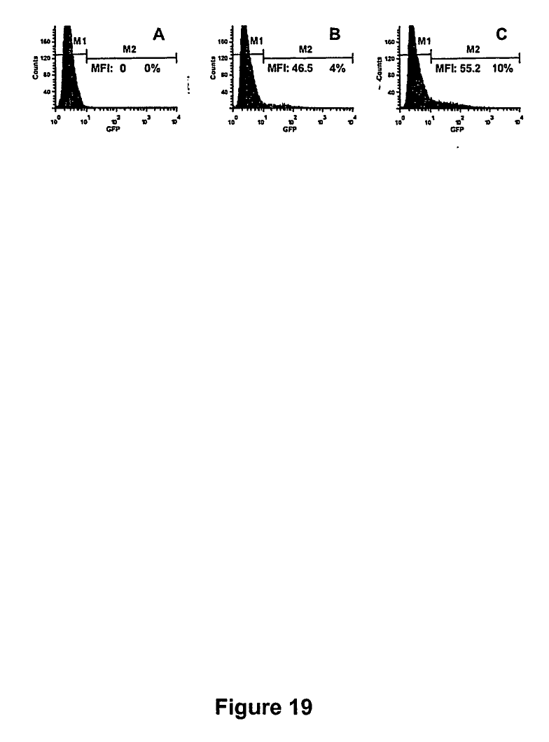

To determine if the ability of the episomal chimeric vectors to maintain high levels of transgene expression requires functional HIV-1 integrase, chimeric EIAV/HIV-1 vectors were packaged with either the traditional HIV-1 packaging system, or with the integrase-deficient HIV-1 packaging system pCMVΔInt, in which the point mutation E152A in the integrase catalytic domain renders the HIV-1 integrase inactive. Vector titers were determined by serial dilutions on 293T cells. Vectors were used on 293T cells at MOI of 0.3. Under these conditions, most of the transduced cells contain a single copy of the vector genome (less than 10% of the cells are transduced). At day 3 post-transduction, the mean fluorescence intensity of GFP-expressing cells was determined by FACscan analysis. The level of GFP expression from integrase deficient chimeric vectors (MFI) was comparable to the level of GFP expressed from vTK728 vectors packaged with functional HIV-1 integrase (FIG. 19). These results indicate that a functional HIV-1 integrase is not required for high levels of transgene expression from episomal chimeric vectors.

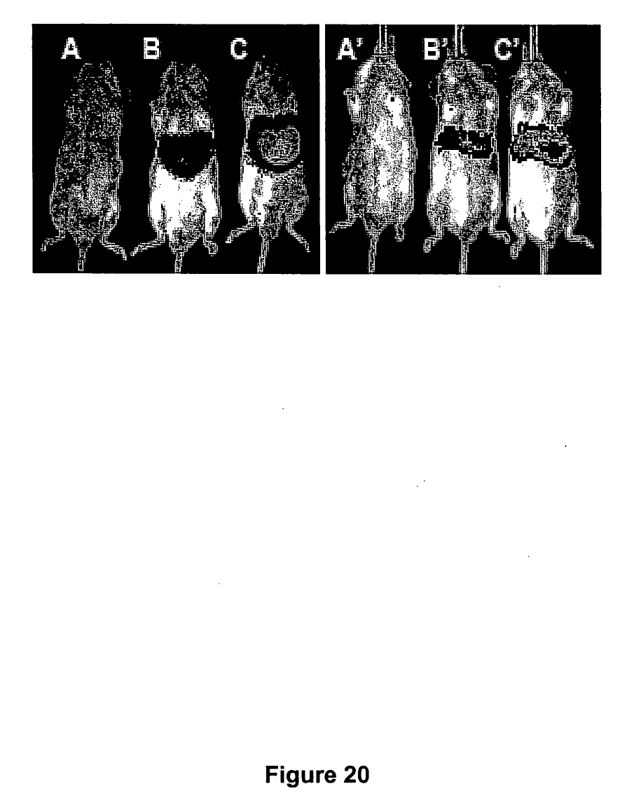

To test the ability of chimeric EIAV/HIV-1 vectors to maintain long-term transgene expression in vivo, two hemophilic mice were injected IP with the 3×10⁸ IU of chimeric EIAV/HIV-1 vector vTK857 (packaged by the HIV-1 packaging system) from which the firefly luciferase is expressed under the control of the liver-specific promoter hAAT (FIG. 20). At weeks 3 and 23 post-injection, luciferase expression was determined in vivo by the Xenogen IVIS Imaging System (Alameda, Calif., United States of America). The episomal EIAV/HIV-1 vector mediated efficient transgene delivery and maintained luciferase expression for more than 5 months. However, some decrease in luciferase expression after 5 months was observed. The decrease could be related to vector dilution due to cell division or to a low level cellular immune response against the luciferase expressing cells. Vector particle titers were determined by p24^{gag} ELISA. Infectious vector titers were determined by qPCR.

The results obtained with the non-integrating chimeric EIAV/HIV-1 vectors prompted characterization of the mechanisms that down regulate transcription from episomal HIV-1 vectors. Because the non-integrating chimeric vectors supported high levels of transgene expression, it is believed that the dilution of non-integrated vector forms upon cell division alone could not account for the low levels of transgene expression obtained from non-integrated HIV-1 vectors. The fact that 5 mM sodium butyrate and 2 mM VPA, potent inhibitors of histone deacetylases, induced a dramatic increase in the transgene expression from integrase mutant HIV-1 vectors (FIG. 19) supported the idea that epigenetic modifications are involved in this phenomenon. Further, these data signified that the episomal vector forms are organized into chromatin structures.

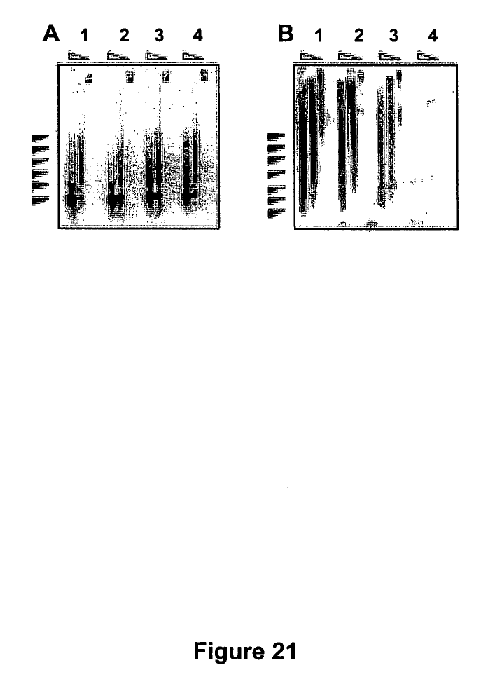

The idea that episomal HIV-1 vectors are organized into chromatin was confirmed by a MNase assay in which exposure of isolated nuclei to MNase results in the digestion of double stranded DNA exposed between nucleosomes. Typically, treatment of chromatinized DNA with MNase resulted in the formation of a nucleosomes ladder whose size depends on the MNase concentration. As shown in FIG. 21, MNase treatment of nuclei isolated from 293T cells 48H post-transduction, either with a traditional HIV-1 vector or an integrase mutant vector, resulted in the formation of the typical nucleosome ladder. Importantly, MNase treatment of nuclei isolated at day 10 post-transduction resulted in the formation of ladder only in samples obtained from cells transduced with the traditional vector. The dilution of the non-integrating vectors explains the fact that no signal was detected from these vectors at day 10 post-transduction.

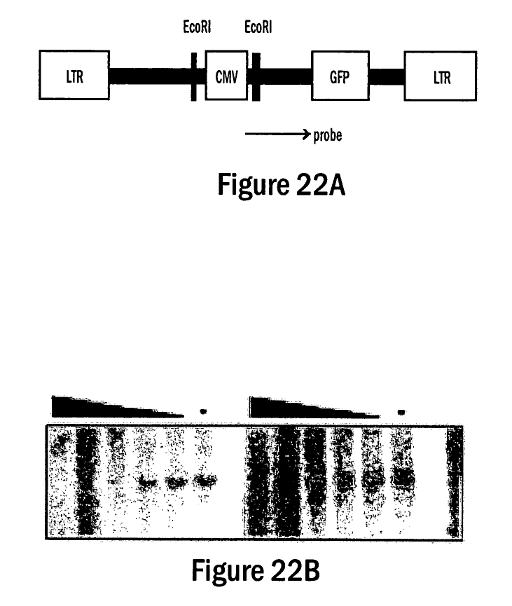

A MNase sensitivity assay (FIG. 21) was used to support the idea that chromatin structure is involved in the silencing mechanism. Nuclei were obtained from cells transduced with the traditional HIV-1 vector vTK113 following five passages in culture, and from cells transduced with integrase deficient vTK113 at day 3 post-transduction. The nuclei were subjected to increasing concentrations of MNase I and the level of digestion was determined by Southern analysis. Episomal vector genomes exhibited significantly higher resistance to DNase I (FIG. 22), which is typical of silent chromatin. Thus, these results support the hypothesis that chromatin modifications are involved in the silencing of episomal HIV-1 vectors.

Specific histone modifications such as H3 and H4 acetylation (Ac H3 and H4) and H3 K4 methylation (H3 K4^{m}) are associated with active chromatin, while H3 K9 methylation (H3 K9^{m}) is typical of inactive chromatin. To characterize the histone modification associated with episomal and integrated lentiviral vectors, a ChlP assay using antibodies specific for Ac H3 and H4 (active chromatin) as well as for H3 K9^{m} (silent chromatin) was used.

In addition, antibodies to H3.1 served as control for histone that does not preferentially associate with transcriptionally active chromatin. Nuclei were isolated from 3 cell populations: a) cells transduced with integrase-deficient HIV-1 vector vTK113 (at day 3 post-transduction), b) cells transduced with traditional HIV-1 vector vTK113 (after 3 passages in culture) and c) cells transduced with chimeric EIAV/HIV-1 vector, packaged with the traditional HIV-1 packaging system.

Chromatin was immunoprecipitated with each of the above antibodies and radioactive PCR was used to amplify a DNA fragment containing the vectors' CMV promoter.

Amplification of the host GAPDH and β-globin promoters served as control for immunoprecipitation of transcriptionally active and silent chromatin, respectively. Immunoprecipitation efficiency of the vector's CMV promoter, using the antibodies to Ac H3 and H4 and H3 K9^{m} was determined as the ratio between the amounts of the PCR products obtained before and after immunoprecipitation and normalized against the values obtained following similar amplification of the GAPDH promoter (when using the antibody to Ac H3 and H4) and the β-globin promoter (when using the antibody to H3 K9^{m}).

Histones associated with active chromatin (Ac H3 and H4) are less likely to bind the episomal HIV-1 vector genome. Importantly, the binding of these histones to the chimeric vector was comparable to their binding to the integrated HIV-1 vector genome. The silent chromatin associated histone H3 K9^{m} was highly efficient at binding to the episomal HIV-1 vector. These results support the hypothesis that differences between the chromatin structure of episomal HIV-1 and chimeric EIAV/HIV-1 vectors might be responsible for some of the differences in gene expression from these vectors.

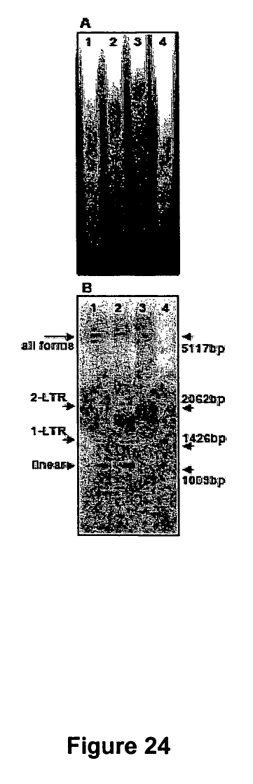

To test the hypothesis that DNA methylation is involved in the silencing mechanism, DNA extracted from vector-transduced HEFs was subjected to CpG methylation analysis using the methylation-sensitive restriction enzyme AaTII. As shown in FIG. 24, vector DNA extracted at day 3 post-transduction from cells transduced with either traditional or integrase-mutant vectors was completely unmethylated (lanes 3,4). As expected, integrated DNA extracted from cells transduced with traditional vector following five passages in culture was also unmethylated and competently digested (lane 2). DNA extracted from cells transduced with integrase-mutant vector and not passaged for 14 days exhibited a restriction digest pattern typical of partially methylated DNA (lane 5). At day 21, episomal DNA in non-dividing HEFs was found completely methylated in all four analyzed AatII sites. These data indicate that DNA methylation is a relatively late process that contributes to the silencing of episomal vectors.

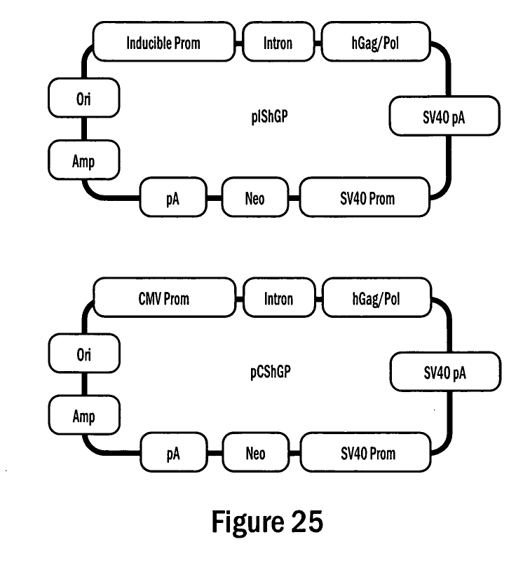

To test the stability of the non-integrating vector, HEFs were transduced with the traditional and the integrase-deficient HIV-1 vector vTK1 13. The cells were cultured for 21 days either with or without being passaged. DNA samples were extracted at day 3 and 21 post-transduction and were subjected to restriction digest and Southern blot analysis. As shown in FIG. 25, in the absence of cell division, the amount of all episomal vector forms (linear, 1-LTR, and 2-LTR) at day 21 was comparable to their amount in day 3. These results indicate that episomal forms are stable in non-dividing cells.

### Example 3

### Effects of Vector cis and trans Elements on Vector Properties

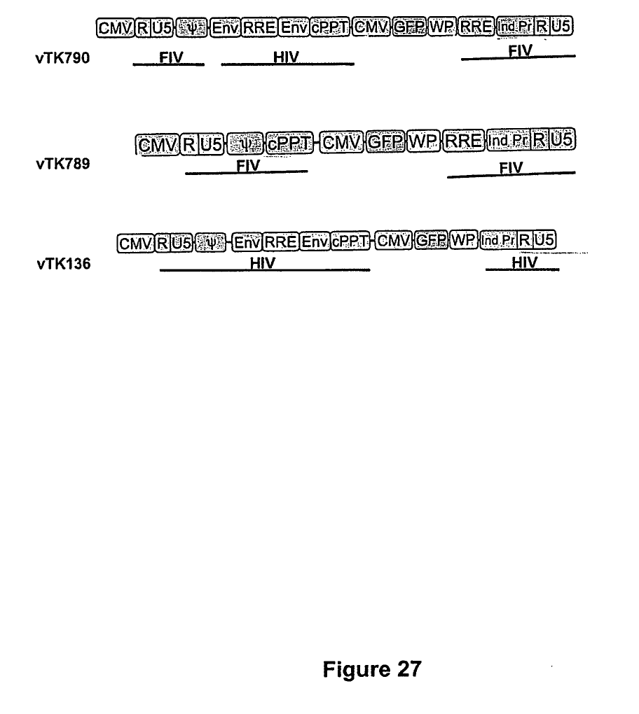

A new 293T cell line (293T-F113) that contains a single copy of the HIV-1 vector TK113 (physical map of the vector included in FIG. 11) was established. The cell line was based on the commercially available Fip-In 273 (Invitrogen, Carlsbad, Calif., United States of America), which contains a single target sequence (FRT site) of the Fip-recombinase. The TK113 vector was cloned into Fip-IN expression cassette to generate the pTK210 construct. Co-transfecting the pTK210 construction with Fip-recombinase expression cassette pOG44 (Invitrogen, Carlsbad, Calif., United States of America), into Fip-IN 293T cells resulted in recombination mediated integration of pTK210 in the FRT site. Single cell clones, resistant to hygromycine were isolated and the presence of a single copy of the TK113 vector was verified by Southern blot analysis (FIG. 27)

### Example 4

### Manipulation of HIV-1 Env/RRE/cPPT Fragment in the FIV Vector

As shown in Example 1, cis elements within the HIV-1 Env/RRE/cPPT fragment are culpable for the ability to efficiently package FIV, MLV, and EIAV vectors with HIV-1 trans packaging elements. The FIV vectors were competent for integration following transduction of target cells. Manipulation of the HIV-1 cis elements is executed in the context of a cross-packaged FIV vector.

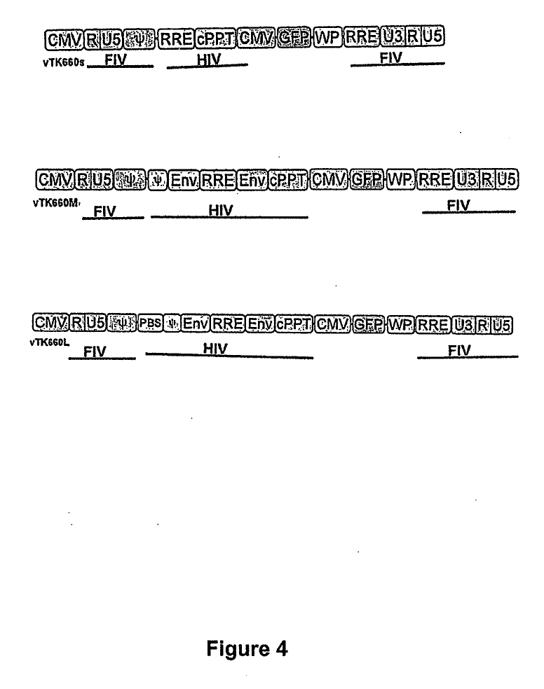

The HIV-1 Env/RRE/cPPT fragment comprises a significant amount of the HIV-1 envelope gene (approximately 140 bp 5′ of the RRE and approximately 475 bp 3′ of the RRE) (SEQ ID NO:2). To minimize putative recombination events between the FIV vector and HIV-1 packaging elements, and to identify cis elements relevant to packaging, this region is constricted to only the RRE. Standard PCR techniques are used to amplify the RRE region of the HIV-1 Env/RRE/cPPT fragment (FIG. 4, depicting construct pTK660S). This is subcloned into the NotI/PpuMI sites of pTK660, replacing the existing Env/RRE/cPPT.

Vectors are generated and tested on 293T cells. The eGFP expression levels are evaluated by FACS analysis and vector integration assessed by qPCR executed on genomic DNA using primers directed to the WPRE. Expanding upon the HIV-1 Env/RRE/cPPT fragment includes the HIV-1 cis elements comprising the packaging signal (ψ) and the primer binding site. The characterized Env/RRE/cPPT fragment is extended by subcloning the fragment containing the HIV-1 packaging signal or the packaging signal and primer-binding site into the corresponding FIV vector (FIG. 4, depicting constructs pTK660M and pTK660L).

These characterized HIV-1 cis elements are then introduced into an FIV SIN vector. The FIV SIN vector conveys an added level of safety, thereby reducing the possibility of generating replication competent vectors. The novel FIV SIN vector containing the most favorable HIV-1 cis elements and control vectors (including the parental non-SIN FIV vector) are generated and tested.

### Example 5

### Evaluation of the HIV-1 RRE for Nucleocytoplasmic Transport and Subsequent Encapsidation

To demonstrate that nucleocytoplasmic transport of full-length MLV vector mRNA transcripts functions independent of the HIV-1 RRE during vector production, nuclear and cytoplasmic extracts are prepared from an HIV-1 plasmid (pTK113, FIG. 11) as a positive control. Total RNA is isolated form each of the nuclear and cytoplasmic extracts and subjected to fractionation via agarose gel electrophoresis, and subsequent Northern blot analysis. Blots are hybridized with a α³²P labeled cDNA probe directed to the internal CMV promoter to impart identification of full-length transcripts expressed from the MLV vectors (pTK493 and pTK494, FIG. 11), as well as the control HIV-1 vector (pTK113). Vectors are harvested from duplicate transient co-transfections to evaluate the encapsidation of full length vector mRNA transcripts by Northern blot analysis and RNA levels are determined by quantitative RT-PCR with primers that amplify a region within the CMV promoter. The packaging efficiency is assessed by eGFP expression in 293T cells via FACS analysis. Similar experiments are performed for FIV and EIAV cross-packaged vectors to test the requirement for the HIV-1 RRE/Rev system on nucleocytoplasmic transport and encapsidation of full-length lentiviral vector transcripts.

### Example 6

### Contribution of the MLV Packaging Signal to Cross-Package With the HIV-1 trans Packaging Elements

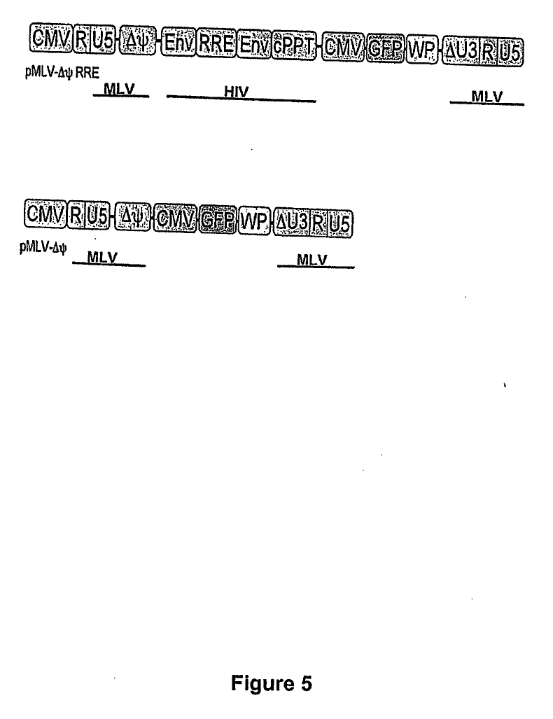

In order to determine if the ψ signal is necessary when cross-packaged in the context of the HIV-1 Rev/RRE system, a deletion of the MLV packaging signal is incorporated into pTK493 and pTK494, while retaining the dimerization domains. A subgenomic fragment comprising the MLV ψ signal and dimerization domains is removed from pTK506 (MLV vector, FIG. 11) with AscI/AgeI and subcloned into pSL301 (Invitrogen, Carlsbad, Calif., United States of America). Standard PCR techniques are used to amplify a region containing the MLV dimerization domains 5′ to the MLV ψ signal, eliminating a major portion of the ψ signal. The product is cloned into a TA cloning vector (Invitrogen, Carlsbad, Calif., United States of America), sequenced and subcloned into pTK493 and pTK494 to generate pMLVΔψ and pMLVΔψ/HIVRRE, respectively (FIG. 5). The novel Δψ constructs, as well as precursor constructs, are cross-packaged with the HIV-1 ΔNRF construct. Levels of eGFP expression are monitored using FACS analysis on 293T cells at 4 days post-transduction. Additionally, nucleocytoplasmic transport and encapsidation of full-length vector mRNAs from the Δψ constructs and the antecedent constructs are examined by Northern blot analysis and quantitative RT-PCR.

### Example 7

### Evaluation of Vector Particles for the Presence of Encapsidated mRNA Transcripts Expressed from the ΔNRF Packaging Construct

In the event that the HIV-1 Rev/RRE cis/trans elements are sufficient for encapsidation in the absence of additional cis elements, it is reasonable to surmise that mRNA transcripts from the HIV-1 ΔNRF packaging construct are encapsidated into vector particles. The vector particles are examined for the presence of ΔNRF mRNA transcripts using Northern blot analysis and quantitative RT-PCR.

Vector particles are prepared from 293T cells co-transfected with ΔNRF and VSV-G plasmids. To test that full-length vector transcripts can compete with ΔNRF for encapsidation when present in the transfection, vector particles are also prepared form HIV-1 pTK113 as a control. Northern blots of total vector RNA are analyzed with a probe to the HIV-1 reverse transcriptase sequence. Quantitative RT-PCR is executed with primers that amplify a region within the HIV-1 RT. Total RNA isolated from 293T cells at 48 hours post-transfection serves as a control for detection of RT sequence by Northern blot analysis and quantitative RT-PCR.

### Example 8

### Generating MLV and EIAV Cross-Packaged Vectors That Are Integration Competent in Target Cells

Integration of the MLV-based vector cross-packaged with HIV-1 trans packaging elements is pursued through replacement of MLV att sites (GACGGGGGTCTTTCATT and AATGAAAGACCCCACCTG)(SEQ ID NOs: 5 and 6, respectively) with HIV-1 att sites. Two oligonucleotides are synthesized, with each comprising the HIV-1 U5 att (GTGGAAAAATCTCTAGCAGT)(SEQ ID NO:7) or U3 att region (ACTGGAAGGGCTAATTTGGTCCGAA)(SEQ ID NO:8), while retaining adjacent 5′ and 3′ MLV sequence. The HIV-1 U5 att sequence is subcloned into a subgenomic fragment of the MLV 5′ LTR. The HIV-1 U3 att sequence are subcloned into a subgenomic region of the MLV 3′ LTR. All sequences are confirmed by sequence analysis.

Subsequently, the HIV-1/MLV chimeric 5′ and 3′ LTRs are introduced individually into the parental MLV vector which lacks the HIV-1 Env/RRE domain, or which harbors the HIV-1 Env/RRE region. Accordingly, six HIV/MLV chimeric vectors are constructed comprising a 5′ HIV/MLV chimeric LTR and a 3′ MLV LTR +/−HIV-1 Env/RRE, a 5′ MLV LTR and 3′ HIV/MLV chimeric LTR +/−HIV-1 Env/RRE, or a 5′ HIV/MLV chimeric LTR and 3′ HIV/MLV chimeric LTR +/−HIV-1 Env/RRE. Each of the vectors are packaged with either the MLV or Gag-Pol packaging cassette or ΔNRF.

All of the vectors are assessed on 293T cells for the capacity to integrate using fluorescence microscopy to score cells for eGFP expression at 4 and 21 days post-transduction. At 21 days post-transduction, the 293T cells are subcultured at least 5 times, thus permitting dilution of any non-integrating vector and a reduction in eGFP expression. Integrating vector are assayed by qPCR at 4 and 21 days post transduction. qPCR are executed on genomic DNA using primers designed to the WPRE. FACS analysis is used to assay 293T cells for levels of eGFP expression at 4 and 21 days post-transduction.

### Example 9

### Integration-Competent EIAV Vector When Cross-Packaged with the HIV-1 trans Packaging Elements

Domain mutagenesis is used for altering the EIAV att sites. A PacI/HindIII fragment encompassing the EIAV att site (5′ GTTCGAGATCCTACAGT 3′)(SEQ ID NO:9) in the U5 region of the 5′ LTR is subcloned into a standard cloning vector (pNEB193, New England Biolabs, Ipswitch, Mass., United States of America). The BglII/EcoRI fragment comprising the EIAV att site (5′ ACTGTGGGGTTTTTATGAG 3′)(SEQ ID NO:10) in the U3 region of the 3′ LTR is subcloned into the standing cloning vector pBlueScript (Strategene, Cedar Creek, Tex., United States of America). Mutagenesis of each of the EIAV att sites ensues, using oligonucleotides designed the change the U3 att (5′ caaggggggaACTGGAAGGGCTAATTTGGTCCGAAgggttttatac 3′)(SEQ ID NO:11) and the U5 att (5′ gtctctagtttgtcGTGGAAAAATCTCTAGCAGTtggcgcccgaac 3′)(SEQ ID NO:12) according to the QuikChange XL Site-Directed Mutagenesis (Stratagene, Cedar Creek, Tex., United States of America) protocol.

Two plasmids harboring the chimeric EIAV/HIV U5 region and EIAV/HIV U3 region result. Each plasmid is sequenced to confirm the exchange of the att sites. Subsequently, the HIV-1/EIAV chimeric 5′ and 3′ regions are introduced individually into the EIAV vector which lacks the HIV-1 Env/RRE domain, or a plasmid which harbors the HIV-1 Env/RRE region (pTK728). Accordingly, six HIV-1/EIAV chimeric vectors are constructed comprising a 5′ HIV/EIAV chimeric LTR and 3′ EIAV LTR +/−HIV-1 Env/RRE, or a 5′ HIV/EIAV LTR and 3′ HIV/EIAV chimeric LTR +/−HIV-1 Env/RRE, or a 5′ HIV/EIAV chimeric LTR and 3′ HIV/EIAV chimeric LTR +/−HIV-1 Env/RRE. Each of the vectors is packaged with ΔNRF or the EIAV packaging cassette.

### Example 10

### Molecular Mechanism That Imparts Efficient Packaging of EIAV/HIV-1 Vectors into HIV-1 Particles

The minimal HIV-1 packaging cassette and the VSV-G fusogenic envelope gene can be stably incorporated into a 3^{rd} generation lentivirus vector packaging cell line yielding high titer vectors.

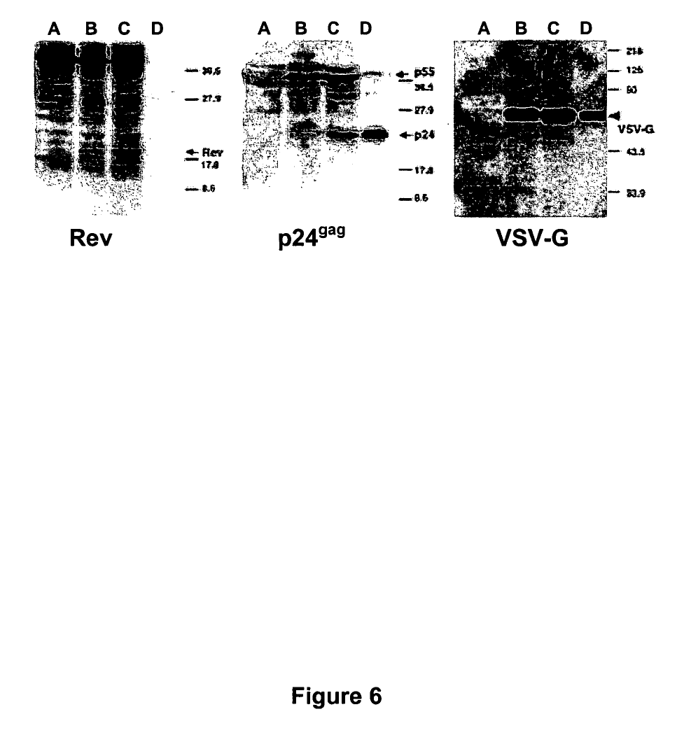

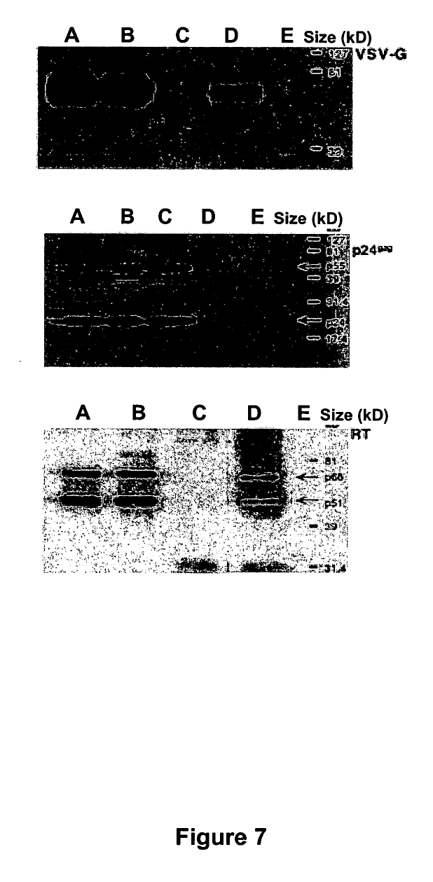

Three packaging cell lines are established: a) the conditional self-inactivating SODk-1 (Xu, K. et al. (2001) Mol Ther 3:97-104), b) the third generation packaging cell line WAN-1 (See Xu, K. et al. supra), and c) the split gag/pol packaging cell line SODk-3. In addition, two vectors are developed, the conditional-SIN vector (See Xu et al., supra) and the IRES based vector, which facilitate the incorporation of lentiviral vectors into stable packaging cell lines and allow selection of high vector producing populations of vector packaging cells. As noted in FIGS. 6 and 7, the packaging cell lines are based on the tetracycline inducible system, which efficiently controls the expression of the toxic VSV-G envelope and the HIV-1 protease.

The WAN-1 cell line is a third generation packaging cell line from which all of the HIV-1 accessory genes have been deleted. Each of the four expression cassettes (Rev, gag/pol, VSV-G, vector) are incorporated sequentially into the cell line by stable transfection. This Tat-deficient cell line produces high titer VSV-G vector particles (10⁹ IU/mL).

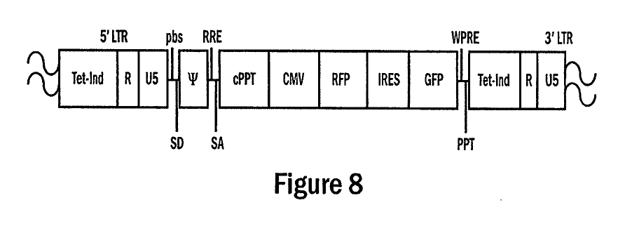

The SODk-3 cell line is the first retroviral vector packaging cell line in which the gag and pol expression cassettes are separated. To incorporate the Pol protein into the vector particle, it is fused to the Vpr. Interaction of the Vpr with the HIV-1 p6^{gag} results in efficient incorporation of the Pol protein into the HIV-1 vector particles. As shown in FIG. 7, the SODk-3 packaging cell line is based on the tetracycline inducible system, which efficiently controlled the expression of the toxic VSV-G envelope and the HIV-1 protease. To facilitate the isolation of the high vector producer cells, an IRES-GFP cassette is incorporated into the conditional SIN vectors (FIG. 8).

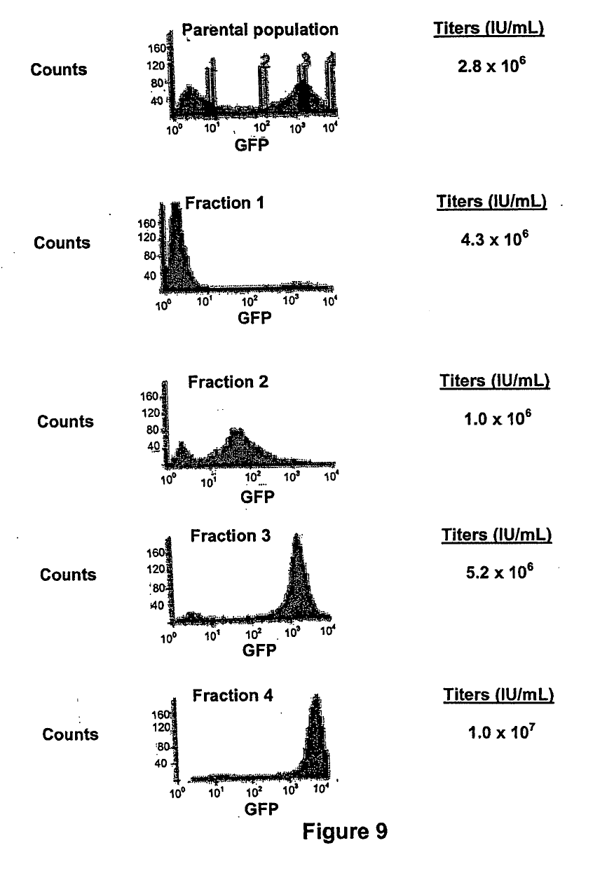

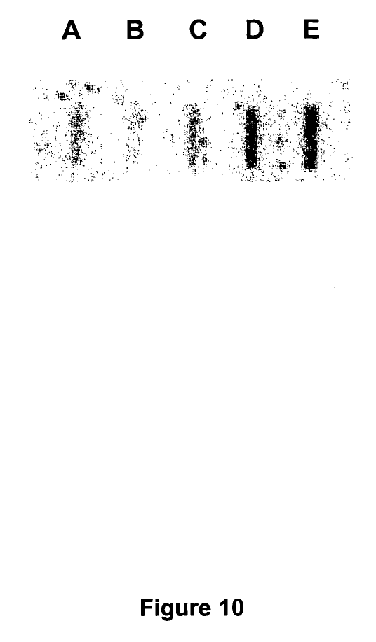

As shown in FIG. 9, using fluorescence-activated cell sorting (FACS) a correlation between the levels of transgene expression in a population of packaging cells to the vector titer obtained from this particular population is shown. Further, slot blot analysis on vector particles obtained from each fraction show a correlation between the levels of packaged vector RNA per vector particles (p24), the level of GFP expression in the different fractions and the titers obtained from these fractions (FIG. 10)

### Example 11

### Evaluation of Whether Rev-Dependent Nuclear Export of Unspliced mRNA is Sufficient for Packaging of the Chimeric Vectors

In order to test whether a specific Rev function other than nuclear export of unspliced mRNA (gag/pol and vector) is required for cross-packaging of MLV/HIV-1 vectors, the ability of Rev mutants lacking specific Rev functions to support efficient packaging of the chimeric MLV/HIV-1 vectors is tested. To allow efficient gag/pol expression in the absence of Rev, the Rev/RRE independent packaging construct pcDNA3.g/p4CTE, from which the HIV-1 gag/pol are expressed under the control of a CMV promoter is utilized. The expression cassette neither encodes the HIV-1 Rev nor contains the RRE sequence.

Four copies of the Mason-Pfizer monkey virus constitutive transport element (CTE) located upstream to the PolyA site, mediate efficient nuclear export of the gag/pol mRNA. As shown herein, the MLV-based vectors vTK493 and vTK494 are packaged efficiently by the traditional MLV packaging system in the absence of Rev. To allow efficient Rev-independent nuclear export of EIAV based vector mRNA, a klenowed SalI/XhoI DNA fragment containing the CTE is cloned into a klenowed ClaI site in pTK728 to generate pTK728c.

Based on this approach, the effects of different Rev mutants on the efficiency of chimeric EIAV/HIV-1 and chimeric MLV/HIV-1 vectors to cross-package into HIV-1 particles is examined. To this end, the traditional HIV-1 vector vTK113 (FIG. 11), the chimeric MLV/HIV-1 vector vTK494 (FIG. 11), the traditional MLV vector vTK493 (FIG. 11), and the chimeric EIAV/HIV-1 vector vTK728c (FIG. 16) are generated by transient transfection, using either the pcDNA3.g/p4CTE packaging cassette (for all vectors), or the traditional MLV-gag/pol expression cassette (for the MLV based vectors), or the traditional EIAV packaging (for vTK728).

All the vectors express the GFP marker gene under the control of the CMV promoter. Vector packaging is carried out either in the absence of the HIV-1 Rev or in the presence of different Rev proteins, including WT-Rev, Rev lacking the RNA binding domain, Rev^{38,39G}, Rev lacking the multimerization domain, Rev^{27-27A} (Zapp, M. L., et al. (1991) Proc Natl Acad Sci USA 88:7732-7738), Rev lacking functional nuclear localization domain, pM4, and Rev lacking the nuclear export function, pM10 (Malim, M. H., et al. (1989) Cell 58:205-214).

Vector particles are generated by transient EIAV packaging cassettes, vector cassettes and VSV-G envelope cassettes. In addition the relevant Rev expression cassette is added to the transfection mixture. Vector particles are collected. Concentration of HIV-1, MLV, and EIAV physical particles are determined by either p24^{Gag} ELISA (for HIV-1 only) or RT assays.

To test the effect of the WT and the different Rev mutants on the efficiency of cross-packaging, equal amounts of vector particles are used on 293T cells. Efficiency of transduction and level of GFP expression are determined by scoring GFP expression following serial dilution and by FACscan analysis.

Infectious titers are determined by qPCR using DNA extracted from vector transduced cells. The FLP9 cell line contains a single copy of the HIV-1 vector vTK113 and served as a reference to establish the standard curve. PCR assay is based on primers directed to the WPRE sequence, which was incorporated to all of the viral constructs described herein. A DpnI restriction site flanked by the WPRE primer sequences are used to eliminate plasmid carryover prior to the qPCR reaction. Efficiency of packaging into vector particles is determined by slot-blot analysis using equal amounts of physical vector particles as determined by RT assay.

A ³²P labeled DNA fragment containing the GFP sequence are used as a probe. The relative amount of mRNA in each sample is quantified by phosphoimaging. To determine the efficiency of nuclear export of packageable vector mRNA, Northern blot analysis of nuclear and cytoplasm RNA extracted from vector-producing 293T cells at 48 h post-transfection is used. A ³²P labeled DNA fragment containing the packaging signal of either the HIV-1 vector, the EIAV-based vectors of the MLV-based vectors is used as probes. The ratio of the cytoplasmic to nuclear mRNA is determined after phosphoimaging quantification.

In a different approach to determine the role of the RRE/Rev as an alternative packaging signal, the effects of leptomycin B on the production of the CTE-containing chimeric EIAV/HIV-1 vector vTK728c, and the chimer MLV/HIV-1 vector vTK494 is tested. Leptomycin is an antibiotic compound that inhibits RRE/Rev-mediated mRNA export by disruption Rev/CRM 1 interactions (Otero. G. C., et al. (1998) J Virol 72:7593-7597). Thus, leptomycin B can block a potential Rev-mediated increase in nuclear export of vTK728c and vTK494 mRNA.

### Example 12

### Specificity of Chimeric EAIV/HIV-1 Cross-Packaging

Because the chimeric EIAV/HIV-1 vector vTK728 contains the EIAV RRE sequence, it is believed that the EIAV Rev can mediate efficient nuclear export of the vTK728 (FIG. 17) mRNA. Chimeric EIAV/HIV-1 vector are produced by transient transfection, using the pcDNA3.g/p4CTE packaging cassette in the presence of either the EIAV Rev (pRS-Erev AIDS Research and Reference Reagent program, cat # 4200) or the HIV-1 Rev, or in the absence of Rev expression cassette.

Vector titers and levels of transgene expression are determined by scoring GFP expression following serial dilutions on 293T cells, and by FACscan analysis, respectively. In addition, titers of infectious particles are determined by qPCR, suing DNA extracted from vector transduced cells. The efficiency of vector packaging is determined by Northern slot blot using vector mRNA extracted from equal amounts of vector particles as determined by RT assays. Northern blot analysis using either cytoplasmic or nuclear mRNA extracted from the vector-producing 293T cells as 48 hour post-transfection is employed to determine nuclear export efficiency of the chimeric vector mRNA. A ³²P labeled DNA fragment containing the EIAV packaging signal serves as a probe. The relative amounts of unspliced full length vector mRNA is quantified by phosphoimager. Probing for GAPDH mRNA serves as a control for mRNA fractionation.

### Example 13

### Minimal HIV-1 Sequence Required for Efficient Chimeric EIAV/HIV-1 Vector Production

Chimeric EIAV/HIV-1, as well as traditional HIV-1 vectors containing a series of deletions in the Env/RRE sequence are provided and the effect of these deletions on vector titers and transgene expression is determined.

To map the regions in the RRE/env which are required for either packaging or nuclear export of vector, new traditional HIV-1 and chimeric EIAV/HIV-1 vectors in which either or both the 5′ and 3′ env sequence flanking the HIV-1 RRE are deleted are provided. In addition, a traditional HIV-1 and chimeric EIAV/HIV-1 vector in which the Rev binding region is deleted is provided.

To this end, DNA fragments containing either the RRE+ 3′ env sequence (Δ5), the RRE+5′env sequence (Δ3), or the RRE only (Δ35) are amplified by PCR. The PCR products in Δ5, Δ3, and Δ35 are digested with NotI/BamHI and cloned into identical sites in pTK113 to generate the new HIV-1 vector pTK113 Δsii.

To generate the homologous chimeric EIAV/HIV-1 vectors, NotI/SacII DNA fragments from the above vectors are cloned into identical sites in pTK728 to generate the chimeric vectors pTK728 Δ5, ptk728 Δ3, pTK728 Δ35, and pTK728 ΔSII, respectively. The above new vector constructs, as well as the parental constructs pTK113 and pTK728, are used to generate vector particles by transient transfection using the pcDNA3.g/p4CTE packaging cassette.

All the transfection is carried out either in the presence or absence of Rev (pRSV-Rev, Cell Genesys—Foster City, Calif., United States of America). Vector titers and levels of transgene expression are determined by scoring GFP expression following serial dilutions on 293T cells and by FACscan analysis, respectively. Infectious titers are determined by qPCR on DNA samples extracted from vector-transduced 293T cells.

Packaging efficiency is determined by Northern slot blot analysis, using vector mRNA extracted from equal amounts of vector as a probe. Northern blot analysis using either cytoplasmic or nuclear mRNA extracted from the vector-producing 293T cells at 48 h post-transfection is used to determine nuclear export efficiency of the chimeric vector mRNA. A ³²P labeled DNA fragment containing the WPRE sequence serves as a probe. Northern blot analysis using either cytoplasmic or nuclear mRNA extracted from the vector-producing 293T cells at 48 h post-transfection is used to determine nuclear export efficiency of the chimeric vector mRNA. A ³²P labeled DNA fragment containing either the HIV-1 or the EIAV packaging signal serves as a probe. Probing for GAPDH mRNA serves as a control for mRNA fractionation.

### Example 14

### Effects of the EIAV Packaging Signal on Cross Packaging of Chimeric EIAV/HIV-1 Vectors

To characterize the effects of parental EIAV packaging signal on the efficiency of cross-packaging of the chimeric EIAV/HIV-1 vector, chimeric vectors lacking a functional EIAV packaging signal are provided.

An EcoRI/PvuII DNA fragment (of 350 bp) is deleted from the EIAV packaging signal in the chimeric EIAV/HIV-1 vector construct pTK728 to generate the packaging signal-deleted chimeric EIAV/HIV-1 vector pTK728 Δψ by transient transfection using either the traditional EIAV or the traditional HIV-1 packaging systems.

Vector titers and levels of transgene expression are determined by scoring GFP expression following serial dilutions on 293T cells, and by FACscan transduced 293T cells. Packaging efficiency is determined by qPCR on DNA samples extracted from vector-mRNA extracted from equal amounts of vector particles as determined by RT assays. A ³²P labeled DNA fragment containing the WPRE sequence serves as a probe.

### Example 15

### Molecular Mechanisms that Impart Efficient Packaging of EIAV/HIV-1 Vectors and Render Them Resistant to Transcriptional Silencing While in Non-Integrated Form

Chimeric EIAV/HIV-1 vectors that are efficiently packaged by the HIV-1 system failed to integrate, and yet maintain a high level of transgene expression. This feature can distinguish the chimeric EIAV/HIV-1 vector from other non-integrating lentiviral vectors, such as integrase mutant HIV-1 vectors.

To determine the effect of host factors on the ability of chimeric EIAV/HIV-1 vectors to express high levels of transgene, transgene expression in various cell types from chimeric EIAV/HIV-1 vectors packaged with either EIAV or HIV-1 packaging systems are analyzed. The chimeric vector expresses either the GFP or Gaussia luciferase from the CMV promoter. The cell lines human 293T, HEF, HeLa, HepG2, primary human hepatocytes, primary human hepatic stem cells, N6 equine cell line, MEF, primary mouse sympathetic ganglia cells, primary mouse bone marrow derived dendritic cells, NmuLi mouse liver cell line, and N433 primary feline fibroblasts are transduced. Transgene expression is determined at day 4 post-transduction by FACscan analysis for GFP expression or quantified by luciferase expression assay.

To determine whether HIV-1 integrase activity protects the episomal chimeric form from transcriptional silencing, an HIV-1 integrase deficient packaging system is used. Traditional HIV-1 and chimeric EIAV/HIV-1 vector carrying GFP or Gaussia luciferase are packaged either with the traditional HIV-1 packaging system or the HIV-1 integrase deficient packaging system. A normalized amount of vector particles is used to transduce 293T cells and HEFs.

The effect of the EIAV parental packaging signal is examined. Serial deletions are made in the parental EIAV packaging signal to determine if it is necessary for HIV-1 RRE/Rev mediated packaging. The chimeric vector and its deleted derivates are packaged by the EIAV and HIV-1 packaging machinery. Normalized amounts of vector particles are tested on 293T cells. Vector titers and transgene expression are determined by scoring transduced 293T cells for reporter expression and by FACscan analysis.

The effect of the att sites are examined to test their effects on vector integration and transgene expression. The EIAV att sites in the chimeric vector with the HIV-1 att sites are replaced. A series of three new chimeric vectors is generated: i) 5′ LTR containing the HIV att site/3′ LTR containing the EIAV att site; ii) 5′LTR containing the EIAV att site/3′ LTR containing the HIV-1 att site; and iii) 5′ LTR containing the HIV-1 att site/3′ LTR containing the HIV-1 att site. Each of these vectors are packaged with the EIAV packaging cassette, the HIV-1 packaging cassette, and the HIV-1 integrase mutant packaging construct. Normalized amounts of vector particles are tested on 293T cells for reporter expression and by FACscan analysis.

HIV-1 is packaged with HIV-1 and HIV-1 integrase mutant packaging systems, and the EIAV/HIV-1 chimeric vector is packaged with HIV-1 and EIAV packaging systems. Normalized vector particles are used to transduce 293T and HEFs at an MOI of 5. At day 3 post-transduction, permeability nuclei from 293T and HEFs is assessed for chromatinization by MNase digestion. Transduced 293T and HEF cells are be cultured and passaged for a period of 3 weeks and the assay is repeated again.

Chromatin immunoprecipitation is used to characterize chromatin modifications associated with integrated and non-integrated vector forms. 293T and HEF cells are subjected to a transduction protocol. At days 3 and 21 post transduction, vector treated 293T cells and HEFs are fixed in a 1% formaldehyde solution and nuclei are isolated and sonicated to generate chromatinized DNA at an average size of 1 kb. Chromatin is immunoprecipitated with antibodies directed against histone H3 (a positive control). For immunoprecipitation of transcriptionally active chromatin, antibodies raised against acetylated H3, acetylated H4, and dimethylated H3-K4 are used. For immunoprecipitation of transcriptionally inactive chromatin, an antibody raised against dimethylated H3-K9 is used. Following immunoprecipitation, DNA is extracted and subjected to qPCR using DNA primers directed to the CMV promoter in the vector cassette and against sequence encoding the human GAPDH gene.

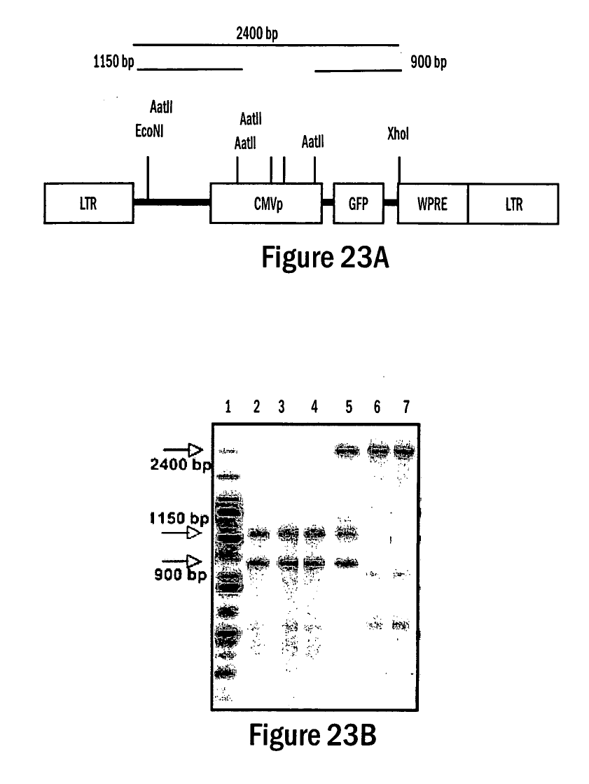

A methylation sensitive DNA restriction enzyme (AatII) is used to characterize the DNA methylation status (FIG. 23). DNA is extracted from vector-transduced 293T and HEF cells at 3 and 21 days post-transduction. DNA is assessed under dividing and non-dividing conditions. The DNA is digested by EcoNI and XhoI in the presence and absence of AatII. Digested DNA fragments is subjected to electrophoresis and Southern blot analysis using a probe directed to the CMV promoter in the vector expression cassette.

### Example 16

### Characterization of the Efficacy and Safety of EIAV/HIV-1 Chimeric Vectors In Vitro and In Vivo

The loss of transgene expressing cells following passages in culture, and the fact that integrated vector sequences could not be detected by Southern blot analysis indicated that most EIAV/HIV-1 vectors did not integrate. However, a cell that maintained transgene expression following several passages in culture was occasionally observed. Thus, a small number of vector genomes that were delivered into the target cells (less than 1% of initially positive cells) integrated into the host genome. The potential for retroviral/lentiviral vectors to integrate within host genes, especially proto-oncogenes, and consequently incite a tumorigenic state is a vector safety issue.

To determine the number of vector genomes and identify the integration site, HIV-1 and EIAV/HIV-1 chimeric vectors expressing a GFP-blasticidin fusion protein reporter are packaged by the HIV-1 integrase mutant system and the traditional HIV-1 packaging system, respectively, to allow comparison of the integrating frequency of integrase mutant HIV-1 vector particles with the integration frequency of EAIV/HIV-1 chimeric vectors. Physical vector particles are normalized by the p24^{gag} assay, and used to transduce genomes at MOI of 10. The number of vector genomes at day 2 post-transduction is determined by qPCR to verify that the target cells were subjected to equivalent amounts of infectious particles. Transduced cells are cultured for 3 weeks and copy number of vector genomes per host cell genome is determined by qPCR. Further, 10⁶ transduced cells are subjected to selection with blasticidin (25 ug/mL). Clones are isolated and separated to identify the integration site using LAM-PCR. Total DNA is extracted from both cell populations and the copy number of vector genomes per host cell is determined using qPCR.

To determine the tumorigenic potential of non-integrating EIAV/HIV-1 chimeric vector, an existing cell line preprogrammed for tumorigenicity is used. The studies use a HMEC line stably expressing genetic elements that perturb most of the p53 and Rb pathways: hTERT (QBiogene, Irvine, Calif., United States of America), p53DD (Shaulian, E., A. Zauberman, D. Ginsberg, and M. Oren (1992) Mol. Cell. Biol. 12:5581-5592), overexpressed cycD1 and mutant R24C allele of the CDK4 gene, and hRAS12V (Finco. T., and Baldwin, A. S., Jr. (1993) J. Biol. Chem. 268, 17676-17679). In these cells, c-Myc function is not altered; therefore these cells have not been antagonized to the point of tumorigenicity.

HMEC5 cells are transduced with varying MOIs of vTK565 (HIV-1 vector) packaged with the traditional HIV-1 system and the integrase mutant system, and vTK565EH (EIAV/HIV-1 chimeric vector) packaged with the traditional HIV-1 system. At 24 hours post-transduction, cells from each transduction are replica plated onto soft agar, monitored for colony formation as a result of anchorage independent growth at 10-21 days post-plating, an indication of tumorigenesis.

Each of the colonies are isolated and clonally expanded to acquire the number of integrated copies necessary to induce tumorigenesis by qPCR and Southern blot analyses. Each colony is assayed for eGFP expression levels by FACscan analysis and quantitative RT-PCR. The heterogeneous population of cells for each transduction is assessed for integrated copy number by qPCR and eGFP expression by FACscan by qRT-PCR.

To test the efficacy of EIAV/HIV-1 chimeric vectors, three reporter assays for in vivo biodistribution and integration studies are used. These assays are based on the expression of GFP, Gaussia luciferase, and a Cre-GFP fusion protein. The GFP reporter supports identification of transduced cells that maintain transgene expression from the episomal vector. The luciferase facilitates live in vivo imaging for assessment of chimeric vector biodistribution, changes in transgene expression over time, and the capacity to quantitate expression. The Cre-GFP reporter is used in combination with a strain of ROSA26 mice containing a LoxP-stop-LoxP-LacZ cassette that remains off until activated by removal of the stop signal with Cre. Expression of the Cre-GFP reporter gene in a target cell of the mice containing a LoxP-stop-LoxP-LacZ cassette results in permanent activation of β-galactosidase expression.

EIAV/HIV-1 chimeric vectors from which the GFP, luciferase, and Cre-GFP are expressed under the control of either CMV or liver specific hAAT promoter are generated. Vector particles are generated by transient cotransfection. Viral particles are harvested at 60 hours post-transfection, purified/concentrated, collected, and resuspended in PBS. Concentration of physical properties is based on p24 ELISA and RT assay. qPCR on DNA from vector transduced 293T cells is used. All vector stocks are tested for replication competent retroviruses by marker rescue assay, p24 transfer assay, and RT transfer assay. Biodistribution studies are executed in Balb/c or ROSA26 strain mice.

### Example 17

### Uniform Retroviral Packaging Cell Line that Imparts Stable Production of High Titer VSV-G Pseudotyped HIV, FIV, and MLV Vectors

Due to the toxicity of the VSV-G envelope and the HIV-1 protease, the basic tTA, Rev expressing cell line is based on the tetracycline inducible system. A cell line expressing the tetracycline regulated transactivator tTA under a CMV promoter and the HIV-1 rev under an inducible promoter, has been established, and was successfully used to establish the third generation packaging cell line.

To facilitate Rev-independent expression of the Gag-Pol packaging cassette, a humanized Gag-Pol packaging cassette under the control of a tetracycline inducible promoter is provided to allow for exclusion of the RRE from the Gag-Pol sequence, and reduction in homology between the packaging and the vector constructs. To further improve the biosafety of the new cell line, the packaging construct is devoid of all HIV-1 accessory genes.

The humanized Gag-pol expression cassette maintains the parental gag-pol amino acid sequence. However, the AU rich HIV-1 mRNA sequence has been optimized according to the codon usage of human genes. The humanization of the Gag-Pol sequence was executed by a software program (described in Anson, D. S. et al. (2005) J Gene Medicine 11:1390-1399) that further optimizes translation efficiency by minimizing likelihood of generating unfavorable secondary mRNA structures. All INS in the HIV-1 Gag-Pol mRNA are modified to increase mRNA stability. A Kozak sequence has been included in the packaging construct to optimize translation initiation. A pUC based plasmid containing the humanized Gag-Pol sequence is synthesized. Synthesis services are available from BlueHeron, Inc. of Bothell, Wash., United States of America.

To test the ability of the humanized Gag-Pol coding sequence to support efficient vector production, two expression cassettes are developed from which the HIV-1 Gag and Gag-Pol mRNAs are expressed under either a constitutive CMV promoter or a tetracycline inducible promoter (FIG. 25).

A DNA fragment containing the optimized HIV-1 Gag-Pol sequence (including the 5′ Kozak sequence) is cloned into pCI-neo (Promega, Madison, Wis., United States of America). An oligonucleotide containing a BglII recognition site is cloned into a pCI-neo to generate cDI-neoB. The tetracycline inducible expression cassette is generated by replacing a BglII DNA fragment containing the CMV promoter in pCI-neoB with a DNA fragment containing the tetracycline inducible promoter. The pIShGP construct is generated by cloning a DNA fragment containing the optimized Gag-Pol sequence into similar site in pln-neo (Promega, Madison, Wis., United States of America).

The packaging construct is co-transfected with the HIV-1 vector plasmid pTK113 (FIG. 12, from which the GFP reporter gene is expressed under control of a CMV promoter), the VSV-G envelope expression plasmid, and the Rev expression cassette into 293T cells. The transient transfection procedure is performed in SODk0 cells that constitutively express the tetracycline-controlled transactivator tTA. Northern analysis is used to determine the stability and efficiency at which the optimized Gag-Pol mRNA (which does not contain the HIV-1 RRE sequence) is being exported from transfected cell nuclei in the course of vector production in a Rev/RRE dependent manner.

The overall level of HIV-1 Gag expression and vector particle formation is determined by p24^{gag} ELISA assay of conditioned media obtained at 72 hour post transfection. The level of HIV-1 Pol gene products is determined by an RT assay of conditioned media obtained at 72 hours post-transfection. The processing of the HIV-1 gene products is characterized by using Western blot analysis of producer cell extracts, and concentrated vector particles generated with either the pCShGP, pIShG, or 2-NRF packaging construct. To test the efficacy of the optimized Rev-independent packaging constructs, conditioned media is collected at 72 hours post-transfection and vector titers are determined by scoring GFP expression by fluorescence microscopy following serial dilution of 293T cells.

The inducible packaging construct is incorporated into the SODk-Rev cell line by stable transfection. The selection of the optimal cell clone is based on a marker rescue assay.

To incorporate the inducible packaging expression cassette into SODk-Rev cell line, the pIShGP construct is linearized and transfected by the traditional calcium phosphate method into SODk-Rev in the presence of doxycycline and stable cell clones selected in the presence of G418.

The optimal SODk-RGP cell clone is identified by a marker rescue assay using the conditional SIN vector TK136. To identify the optimal cell clone, each of the isolated cell clones is transduced with TK136 vector at MOI of 5. The transduced cell clones are induced to express the Gag-Pol gene products by withdrawing doxycycline and adding 5 mM sodium butyrate to the culture media. After adding the sodium butyrate, each of the transduced cell clones are transfected with the VSV-G envelope plasmid. Conditioned media is collected and vector titers are determined by scoring GFP expression following serial dilution of 293T cells. The cell clones which produce the highest vector titers are retested for their ability to support high titer vector production.

An inducible VSV-G expression cassette (depicted in FIG. 25) is incorporated into SODk-RhGP cells by stable transfection. The plasmid pBIGFV expresses the VSV-G envelope and GFP reporter gene from a bi-directional tetracycline inducible promoter. The construct is linearized and co-transfected with the puromycin expressing plasmid pPUR (Clontech, Palo Alto, Calif., United States of America) into SODk-RhGP using the calcium phosphate method. Stably transfected cell clones are isolated following selection with puromycin in the presence of doxycycline. Each of the sleeted clones is induced by withdrawal of doxycycline and the addition of 5 mM sodium butyrate. Conditioned media is collected at 3-6 days post-induction and scoring GFP expression on 293T cells will identifies those cell clones that produce the highest vector titers.

cSIN vectors are constructed and incorporated into the SODk-RhPV. To generate the cSIN FIV vector (FIG. 27), a klenowed DNA fragment containing the tetracycline inducible promoter is cloned into the SIN FIV vector. To allow packaging of the cSIN FIV vector by the HIV-1 packaging system, a DNA fragment containing the HIV-1 RRE/Env sequence replaces a fragment in pTK789 to generate the cSIN FIV/HIV-1 chimeric vector pTK790 (FIG. 27). Vector particles are produced by transient three-plasmid transfection and titered by scoring for GFP expression following serial dilution on 293T cells. To generate stable HIV-1 and FIV vector producer cell lines, the SODk-RhGPV cell line is transduced at MOI 5 by the cSIN vectors vTK136 and vTK790 (FIG. 27), respectively.

To determine the efficiency of vector production, the producer cell lines are induced to produce vector particles by the withdrawal of doxycycline and addition of sodium butyrate to the culture media. Samples of conditioned media are collected prior to induction and at days 1-6 after addition of sodium butyrate. Total vector particles in the collected samples are evaluated by p24^{gag} ELISA. Vector titers and the optimal timing for harvesting vector particles is determined by scoring GFP expression following serial dilution on 293T cells. To evaluate the efficacy of vector particles generated by the stable producer cell line, the ratio of maximal vector titer to p24^{gag} concentration in conditioned media is calculated and compared to similar ratios of vector preps produced by the transient three-plasmid method. The efficiency of processing of the HIV-1 gene products is determined by Western blot analysis of vector particles using anti-p24^{gag} antibodies.

To test the ability of the HIV-1 and FIV vectors to transduce non-dividing cells in vivo, conditioned media is collected at 4-6 days post-induction. Vector particles are concentrated by three rounds of centrifugation.

As a control, concentrated lentiviral vector stocks are produced by the traditional three-plasmid transient transfection method and concentrated/purified. Each of the vector stocks is resuspended with PBS to a final concentration. The vectors are injected into the striatum of 12-week-old female fisher rates. At 2 and 12 weeks post-injection, half of the injected animals are sacrificed. Brain tissue is sectioned and efficacy of the packaging cell line derived vectors are evaluated by fluorescence microscopy analysis of GFP expression. The tropism of the injected vectors in rate brain is characterized by immunohistochemistry using antibodies directed against neuron, astrocyte, and microglia specific markers.

### Example 18

### Humanized HIV-1 gag/pol Rev-Independent Stable Packaging Cell Line

To facilitate production of chimeric EIAV/HIV-1 vectors, a humanized gag/pol packaging cell line that yields high titer VSV-G pseudo typed HIV-1 packaged particles is established. The humanization of the HIV-1 gag/pol expression cassette renders the packaging system Rev/RRE-independent. The novel packaging cell line should improve biosafety, facilitate scaling-up vector production, and standardize the production of the chimeric vectors for in vivo studies.

All gene expression cassettes required for vector production (gag/pol, Rev, VSV-G, vector) of the new packaging cell line are driven by a tetracycline-regulated promoter. To substantively exclude the possibility of generating pathogenic RCR, all the HIV-1 accessory genes excluding HIV-1 rev are deleted from the packaging expression system. To reduce the likelihood of generating RCR, the HIV-1 gag/pol and rev sequences are separated onto two expression cassettes. To minimize the risk of recombination between the inducible Rev, Gag/pol, and VSV-G expression cassettes, they are incorporated sequentially three separate stable transfections.

The basal cell line (SODk-Rev) already contains the tTA and HIV-1 rev genes under control of a CMV and tetracycline inducible promoter, respectively. The VSV-G expression cassette is incorporated last to allow flexibility in pseudotyping. To minimize the likelihood of recombination between the packaging and vector expression cassettes, the sequence homology is reduced between the 2 cassettes by deleing the HIV-1 RRE sequence from the Gag/Pol packaging construct. To retain high vector titers, the codon usage of the gag/pol open reading frames is humanized, which renders the expression of the gag/pol gene products rev-independent and further reduce homology between the vector and packaging cassette.

To facilitate Rev-independent expression of the Gag/pol packaging cassette, a humanized Gag/pol packaging cassette under the control of a tetracycline inducible promoter is generated. This will allow for exclusion of RRE from the Gag/pol Sequence, and reduction in homology between the packaging and vector constructs. To further improve biosafety, the packaging construct is devoid of all HIV-1 accessory genes.

The gag/pol sequence is humanized. The modified sequence includes the gag/pol reading frames, excluding the 287 base pair protease frameshift site, in which the HIV-1 Gag and Gag-pol reading frames overlap. The humanized Gag-pol expression cassette maintains the parental Gag-Pol amino acid sequence, but the AU rich HIV-1 mRNA sequence is optimized according to codon usage of humans. The humanization of the Gag-pol sequence was executed by a software program available from BlueHeron Biotechnology, Inc. (Bothell, Wash., United States of America). All instability sequences in the HIV-1 gag-pol mRNA are modified to increase mRNA stability. A Kozak consensus sequence is included in the packaging construct. The pUC plasmid pSyHuGp containing the humanized HIV-1 Gag-pol sequence is synthesized by BlueHeron Biotechnology, Inc.

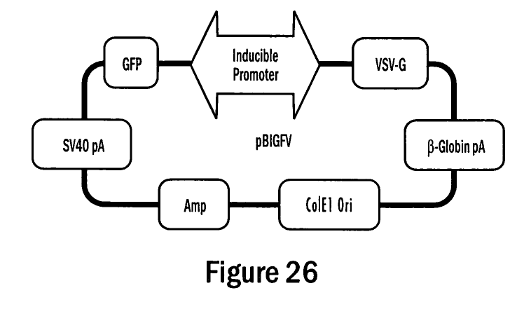

To test the ability of the humanized gag-pol coding sequence to support efficient vector production, two expression cassettes are developed from which the HIV-1 Gag and Gag-Pol mRNAs are expressed under either a constitutive CMV promoter or a tetracycline inducible promoter—pCShGP and pIShGP, respectively (see FIG. 26). An XbaI/NotI DNA fragment containing the optimized HIV-1 Gag-Pol sequence (including the 5′ Kozak consensus sequence) is cloned into XbaI/NotI sites in pCI-neo (Promega, Madison, Wis., United States of America).

An oligonucleotide containing a BglII recognition site is cloned into I-PpoI site in PCI-neo to generate pCI-neoB. The tetracycline expression cassette pln-neo is generated by replacing a BglII DNA fragment containing the CMV promoter in pCI-neoB with a BglII/Bam HI DNA fragment containing the tetracycline inducible promoter.

To characterize the optimized Rev-independent packaging constructs, pCShGP and pIShGP, the ability to support vector production using the traditional three/four plasmid transfection method is evaluated. Optimized packaging construct pCShGP (10ug) is co-transfected with the HIV-1 vector plasmid pTK113 (from which the GFP reporter gene is expressed under the control of a CMV promoter), the VSV-G envelope expression plasmid, pMDG (Zufferey R., Nagy, D., Mandel, R. J., Naldini, L. & Trono, D. (1997) Nat. Biotechnol. 15, 871-875), and the Rev expression cassette into 293T cells. The ability of pIShGP to support vector production is determined in a similar way; however, the transient transfection method is performed in SODk0 cells which constitutively express the tetracycline-controlled transactivator tTA.

As a positive control, TK113 vector particles are produced in a similar way in 293T cells using the non-humanized 3^{rd} generation packaging construct NRF, which expresses HIV-1 gag/pol under the control of a CMV promoter and contains the HIV-1 RRE sequence. Transfected cells and conditioned media are analyzed by Northern analysis, p24^{gag} ELISA assay of conditioned media, RT assay of conditioned media, Western blot analysis of producer cell extracts and concentrated vector particles, scoring GFP expression by fluorescence microscopy of conditioned media.

The pIShGP construct is linearized by PvuI digestion, and transfected by traditional calcium phosphate method into SODk-Rev in the presence of doxycycline. Stable cell clones are selected in the presence of 300 ng/mL G418. The optimal cell clone SODk-RGP is identified by marker rescue assay using conditional self-inactivating vector TK136 (FIG. 26). To identify the cell clone that produce the highest vector titer, the isolated clones are transduced with the TK136 vector at MOI of 5. The clones are then induced to express the Gag-Pol gene products by withdrawing doxycycline and adding 5 mM sodium butyrate into the culture media. Conditioned media is collected and vector titers are determined by scoring GFP expression following serial dilution on 293T cells. The cell clones yielding highest vector titers are tested again for the ability to support high titer vector production. The cell clone that produces the highest vector titer is used to establish the packaging cell line.

An inducible VSV-G expression cassette (pBIGFV, FIG. 26) is incorporated into SODk-RGP cells by stable transfection. PBIGFV expresses the VSV-G envelope and GFP reporter gene from a bi-directional tetracycline inducible promoter. The pBIGFV construct is linearized by restriction digestion with PvuI and co-transfected with the puromycin expressing plasmid pPUR (Clontech, Palo Alto, Calif., United States of America) into SODk-RGP cells using the traditional calcium phosphate method. A total of 30 stably transfected cell clones is isolated following puromycin (1 ug/mL) selection in the presence of 1 ug/mL doxycycline. Each selected clone is transduced with the cSIN vector vTK136 (at MOI of 5). Production of vector particles in the transduced cell clones is induced by withdrawal of doxycycline and the addition of 5 mM sodium butyrate. Conditioned media is collected 3-6 days post-induction and scoring GFP expression on 293T cells identifies the cell cones that produce the highest vector titers. Conditioned media of non-induced cells is used as a negative control and as an approach for evaluating the ability to control vector production. The cell clone that furnishes the highest vector titer and shows tight regulation of vector particles (SODk-RGPV) is used to establish stable HIV-1 and chimeric EIAV/HIV-1 vector producer cell lines.

Cloning of a DNA fragment containing the chimeric EIAV/HIV-1 coding sequence from pTK728 into the expression plasmid pcDNA-Zeo (Invitrogen, Carlsbad, Calif., United States of America) generated the pTK799 construct. This construct is incorporated into SODk-RGPV cells by stable transfection (zeocin selection). The cSIN HIV-1 vector is incorporated into SODk-RGPV cells by transduction. Vector particles are generated by the traditional transient transfection method and are dispensed onto SODk-RGPV cells at MOI of 5. Heterogeneous cell populations of vector containing SODk-RGPV cells (either vTK799 or vTK731), which are likely to produce high vector titers, is isolated by FACS.

To determine the efficiency of vector production, the producer cell lines are induced to produce vector particles by withdrawal of doxycycline and additional on 5 mM of sodium butyrate to the culture media. A sample of conditioned media is collected prior to induction and after addition of the sodium butyrate. Total vector particles in the collected samples are evaluated by p24^{gag} ELISA. Vector titers and optimal timing for harvesting vector is determined by scoring GFP expression following serial dilution on 293T cells. To evaluate efficacy of vector particles generated by the stable producer cell line, the ratio of maximal vector titer (IU/mL) to p24^{gag} concentration (ng/mL) in conditioned media is calculated and compared to similar reactions of vector preparations produced by the transient three plasmid method. The efficiency of processing the HIV-1 gene products is determined by Western blot analysis of vector particles using anti-p24^{gag} antibodies.

To test the ability of the HIV-1 and chimeric EIAV/HIV-1 vectors to transduce non-dividing cells in vivo, conditioned media is collected 4-6 days post-induction and vector particles concentrated. As a control, concentrated lentiviral vector stocks are produced by traditional three-plasmid transient transfection method and concentrated/purified. The control VSV-G pseudotyped, concentrated vector stocks include vTK731 packaged by the HIV-1 packaging construct, and the SIN EIAV vector UNC6.1 packaged by the EIAV packaging system. Each of the four vector stocks are resuspended with PBS to final concentration of 1×10⁹ IU/mL. The vectors are injected into the striatum of 12-week-old female Fisher rats. At 2 and 12 weeks post-injection, half of the mice are sacrificed. Brain tissue is sectioned and efficacy of the packaging cells line derived vectors is evaluated by fluorescence microscopy analysis of GFP expression. The tropism of the injected vectors is evaluated by fluorescence immunohistochemistry using antibodies directed against neuron, astrocyte, and microglia specific markers.

### Example 19

### Effects of the Vector cis and trans Elements on Vector Transduction Efficiency and the Ability to Transduce Non-Dividing Cells

The extent to which the cis and trans vector elements contribute to transduction efficiency is investigated by comparing transduction efficient and level of transgene expression of HIV, FIV and cross-packaged FIV vectors in cell types derived from the human, murine, feline, and equine species. Each of the cell types delineated in Table 1 are used.

TABLE 1

| Cell Type | Species | Source |
|---|---|---|
| 293 T cells | Human | UNC-CH |
| HapG2 cells | Human | ATCC |
| hEF | Human | Kafri lab |
| Primary Mouse neurons | Murine | UNC-CH |
| Primary mouse | Murine | UNC-CH |
| hepatocytes | | |
| NmuLi | Murine | UNC-CH |
| MEF | Murine | UNC-CH |
| N433 | Feline | UNC-CH |
| NSV3 | Feline | UNC-CH |
| NBL-6 | Equine | ATCC |

The vectors employed include HIV-1 TK113 (FIG. 11), cross-packaged FIV (TK660, FIG. 4, +HIV-1 RRE), and FIV TK665 (—HIV-1 RRE) on 293T cells. Vector titers and level of transgene expression are used to determine transduction efficiency. Vector titers are determined, and FACScan analysis and qRT-PCR at day 21 post-transduction are used to evaluate the level of transgene expression.

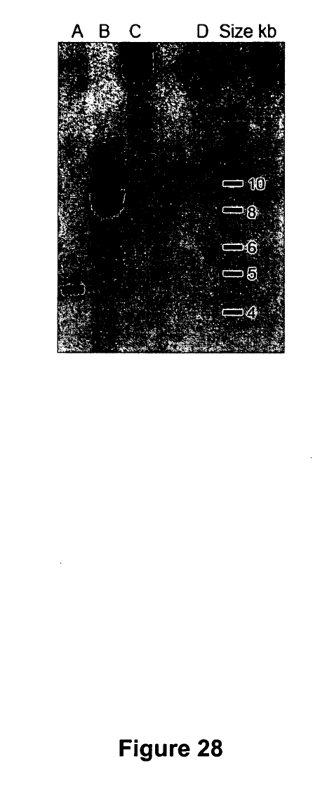

The capacity of cross-packaged MLV vectors to transduce non-dividing cells is tested. First, 293T cells treated with varying concentrations of aphidicolin for 24 hours are used to arrest cells in the G1/S phase of the cell cycle. While arrested, the 293T cells are transduced with the cross-packaged MLV vector and examined for eGFP expression post-transduction. Transduction is assayed by fluorescence microscopy, FACScan analysis, and qRT-PCR with primers to eGFP. Secondly, primary murine neurons are transduced with the above vectors comprising an expression cassette comprising the CMV-IE promoter driving expression of the β-galactosidase gene fused to a nuclear localization signal (FIG. 28). A nuclear localized B-galactosidase protein imparts increased sensitivity in neurons, which would otherwise be highly dispersed throughout the neuron, and potentially undetectable. Primary neurons are assessed for B-galactoside expression post-transduction by staining neurons with X-Gal substrate and scoring for blue neurons by light microscopy. All vectors are titered on 293T cells by scoring for blue cells via phase-contrast microcopy.

### Example 20

### Animal Models For the Efficacy of Lentiviral Vector Gene Delivery In Vivo

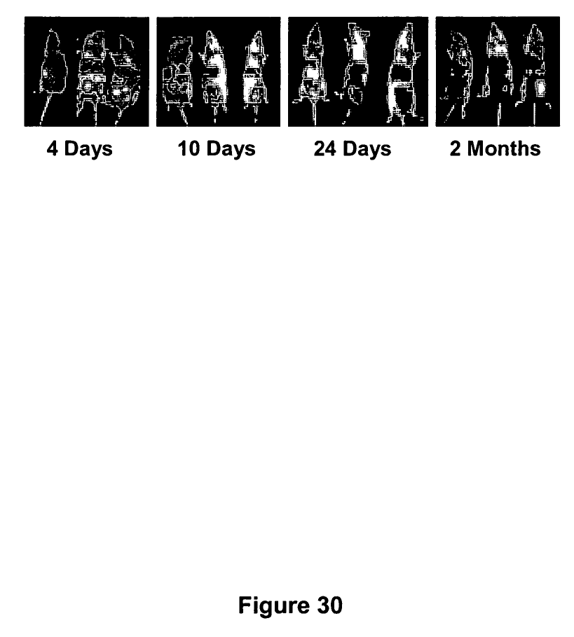

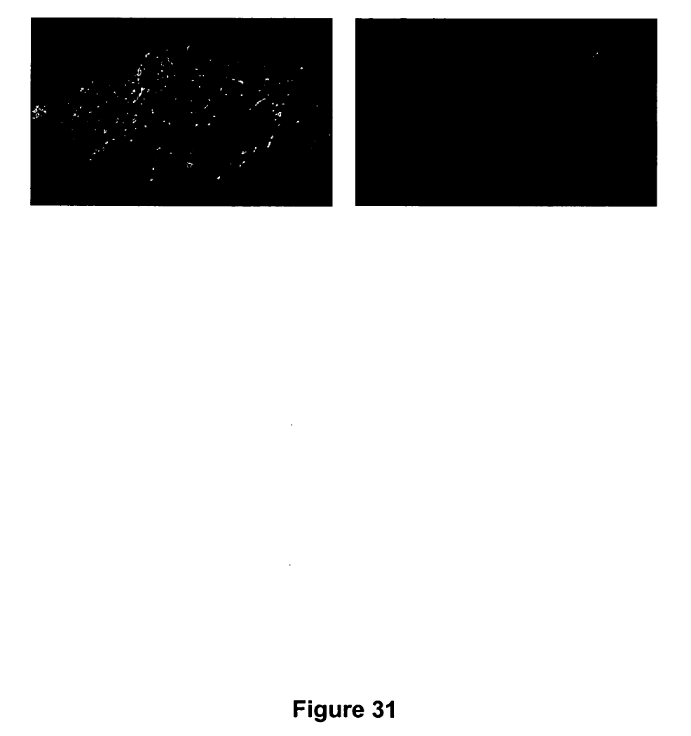

As shown in FIG. 30, IP injection of HIV-1 vectors expressing firefly luciferase gene resulted in efficient transduction of liver tissue in Balb/c mice. Although not as robust as in day four post-injection, transgene expression in the liver of treated animals was stable between day 10 and 2 months (the duration of the experiment). As shown in FIG. 31, efficient transduction of feline cortex with HIV-1 vector resulted in long-term expression of the GFP reporter gene.

Humanized mouse factor IX knockout model of hemophilia B (R333Q-hFIX) has been established (Jin, D. Y. et al. (2004) Blood 104(6):1733-1739). The mouse was generated by a transgenic knockout approach in which a mutated human factor IX allele carrying the missense mutation R333Q replaced the endogenous murine factor IX coding sequence. The R333Q allele also contained the 148Ala form of the human factor IX, which in contrast to the 148Thr form cannot be recognized by the A1 antibody to human factor IX. Although high level of hFIX protein could be detected in R333Q mouse plasma, it activity was less than 1% and the mice exhibited the hemophilia bleeding disorder.

The ability of HIV, FIV and cross-packaged FIV vectors to deliver and maintain long-term transgene expression in murine, rat, and feline animal models is characterized. Comparing the efficacy of the vectors in the animal models allows determination of the relative importance of the interactions between host cell factors and the vector's cis and trans elements for in vivo gene delivery. The vectors are administrated by IP injection.

All the vectors will express the GFP marker gene under the control of either a CMV or hAAT promoter. The vectors are produced by the traditional transient three-plasmid transfection method. The HIV-1 and cross-packaged vectors are packaged by the HIV-1 packaging system and the FIV vector is packaged by the FIV packaging system. A uniform packaging cell line, such as the line disclosed in the Examples hereinabove, is used to produce the HIV-1 and cross-packaged vectors. qPCR using DNA of vector transduced 293T cells is used to determine vector titers. The primers are designed to amplify the WPRE sequence, which is contained in all vectors.

Vectors expressing the GFP under control of the CMV promoter are administered by infusion into the striatum and hippocampus. The animals are euthanized at days 7 and 60 post-injection. One hemisphere of each brain is sectioned (40 microns) and fixed in 4% paraformaldehyde. The level and duration of immunohistochemical co-localization of GFP expression is evaluated by fluorescence microscopy. The transduced target cells are identified by immunohistochemical co-localization of GFP with specific cell markers. The level of inflammation and potential immune response as indicated by the presence of lymphocytes at the injection sites are evaluated using primary antibodies against CD4 and CD8. DNA is extracted from the injection area in the second hemisphere and the number of vector genomes is determined by qPCR.

Vectors expressing GFP under control of the CMV or hAAT promoter are administered by IP injection. Vector administration by IP injection is an efficient and safe route for systemic lentiviral vector delivery, which is used to administer lentiviral vector to hemophilic mice. At days 7 and 60 post-injection, half of the animals are euthanized. Bone marrow and organ samples including liver, spleen, heart, lung, kidney, and ovary are collected. Transduction efficiency and GFP expression levels are determined by fluorescence microscopy on sectioned samples and by qRT-PCR assay, respectively. DNA is extracted from the samples and used to determine vector copy number per host genome at days 7 and 60 post-injection. The distribution of vector-transduced cells within the liver is determined morphologically. The cellular tropism of the various vectors in the liver is determined by immunohistochemistry using antibodies directed to cellular-specific markers. The development of antibodies to GFP is determined by ELISA assay. Blood samples are taken prior to vector administration and once every 10 days. ELISA plates are covered with recombinant GFP (Clontech, Palo Alto, Calif., United States of America) and incubated in serial dilutions of animal sera. GFP specific IgG and IgM antibodies are probed with alkaline phosphatase-conjugated goat anti-mouse, rate or feline IgG and IgM antibodies. The development of cellular immune response against GFP expressing cells is determined by lymphocyte proliferation assays.

### Example 21

### Human Factor IX Expression in Traditional and Humanized Knockout Mouse Models

The development of anti-factor IX immune response and its effect on plasma factor IX concentration in two mouse factor IX knockout models of hemophilia is characterized.

Two lines of factor IX knockout mice are used to test the effects of the animal model design on the outcome of lentiviral vector delivery of human factor IX in a small animal model of hemophilia B. In the first model, the parental factor IX promoter and a portion of the murine factor IX coding sequence is deleted (FIXKO). This strain of mice exhibits the hemophilia B bleeding disorder, but do not express any of a defective factor IX protein which can antigenically cross-react with vector delivered factor IX. In the second model, the murine factor IX coding sequence is replaced with the human factor IX mutant allele R333Q. The R333Q allele contains the alanine form of the human factor IX Ala148Thr dimorphism, which cannot be efficiently recognized by the anti-factor IX anybody A1 (Frazier, D., et al. (1989) Blood 74(3):971-977; Smith, K. J., et al. (1987) Blood 70(4):1006-1013). The R333Q mice exhibit normal plasma level of factor IX antigen yet their clotting function is less than 1% of normal mice.

To test the effects of the animal model design, the origin of the lentiviral vector and the vector's internal promoter on the ability of lentiviral vectors to deliver and maintain factor IX levels in the hemophilia B mice, R333Q and FIXKO mice are infused with lentiviral vectors (HIV-1, FIV, and cross-packaged FIV) from which the human factor IX cDNA (containing the threonine form of the Ala148Thr dimorphism) is expressed under the control of a CMV or hAAT promoter.

All vectors are generated by transient three-plasmid transfection. After establishing a uniform lentivirus vector packaging cell line, it is used to produce the HIV-1 and the cross-packaged FIV vectors. All vectors are titered by qPCR on DNA extracted from vector-transduced 293T cells. Vectors are IP injected into R33Q and FIXKO mice. Blood samples are withdrawn by retro-orbital bleeding prior to vector administration and at days 4, 10, 20, 30, 45, 60 and 90 post-injection. The presence of 148T human factor IX and its concentration is determined by Western blot analysis and ELISA assays using the A1 antibody. The clotting function of factor IX delivered by the different vectors is determined by APTT assay.

The development of inhibitory antibodies is determined by Bethesda assays. The presence and levels of mice IgG and IgM antibodies to human factor IX is determined by ELSIA. At day 45 post-injection, half of the animals are euthanized. Splenocytes are isolated and used for lymphocyte proliferation assay in HL1 media supplemented with human factor IX (Benefix Genetic Institute, Cambridge, Mass., United States of America).

### Example 22

### Use of EIAV/HIV-1 Chimeric Vector in Delivering and Maintaining Factor IX Expression in a Humanized Hemophilic Mouse Model

The ability of episomal EIAV/HIV-1 vectors carrying the hFIX cDNA under the control of the liver-specific promoter hAAT, to deliver and maintain therapeutic levels of hFIX in hemophilia mouse models without inducing hFIX-directed immune response is tested. The development of inhibitory antibodies is a side effect of protein replacement therapy, which constitutes a major obstacle to the treatment of hemophilia A and B patients. Since the nature of the underlying mutation in the dysfunctional gene is a major factor determining the risk of a particular patient to develop inhibitory antibodies, it is useful to characterize the likelihood of a particular viral vector to induce inhibitory-antibody development in animal models, which emulate best the clinical setting of gene therapy protocols.

Episomal EIAV/HIV-1 hFIX vectors are tested in two hemophilia B mouse models. The first model is based on a traditional factor IX KO mouse, which does not express factor IX and thus emulates patients with null mutations that are prone to develop inhibitory antibodies. The second model is based on a humanized hemophilia B mouse R333Q that expresses a human FIX cDNA carrying a single missense point mutation. The protein product of this cDNA is inactive (clotting activity is less than 1%), and yet serves an antigenically cross-reacting material. Thus, similar to human patients, the R333Q mice are less prone to develop inhibitory antibodies.

An efficient episomal chimeric EIAV/HIV-1 vector can alleviate biosafety concerns regarding vector-induced insertional mutagenesis without hampering the efficacy of the lentiviral vector-based immunotherapy approach. Assaying the induction of AV-1 capsid-directed immune responses by the episomal EIAV/HIV-1 vectors facilitates the ability to characterize the immunogenic potential of these vectors.

The efficacy of the episomal EIAV/HIV-1 vectors as a vehicle for factor IX gene replacement therapy in hemophilia B mouse models is characterized. HIV-1 and chimeric EIAV/HIV-1 vectors carrying the human factor IX under the control of the hAAT promoter are administered intravenously (IV) into KO-FIX and R333Q-hFIX mice. Vector dose could affect the development of immune responses, especially following administration integrating hAAT-HIV vectors. Thus, mice are treated with two different does of vectors. Each mouse receives a total of either 2×10⁹ or 1×10¹⁰ IU of the above vectors. Vector titers are determined by qPCR on DNA samples obtained from vector-transduced 293T cells. Blood samples are withdrawn by retro-orbital bleeding at day 7 pre-injection and at days 3, 7, 14, 21, 28, 42, 56, 70, and 91 post-injection and are analyzed as described below for levels/function of human factor IX and for the development of factor IX-directed immune responses.

All the injected lentiviral vectors contain a human factor IX cDNA encoding a fully functioning protein. This cDNA expresses the threonine form of the Ala148Thr human factor IX dimorphism. The R333Q-hFIX mice express the alanine form of the human factor IX. Employing unique antibody A1, which preferentially recognizes the threonine isoform, provides for evaluation of the effectiveness of gene therapy protocols in the humanized hemophiliac mice as described (See, Jin, D. Y.. et al., supra).

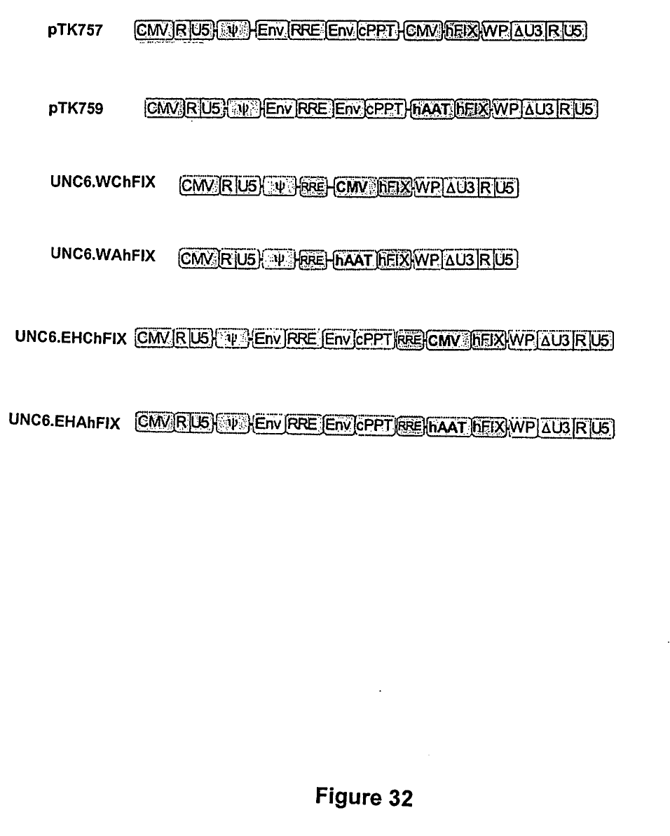

The HIV-1 vector pTK759 from which the human factor IX is expressed under the control of the hAAT promoter shows high levels of factor IX expression in culture (FIG. 32). To generate the chimeric EIAV/HIV-1 vector, a klenowed SacII/EcoNi DNA fragment containing the HIV-1 RRE and the human factor IX cDNA, under control of the hAAT promoter is isolated from pTK759 (FIG. 31), and is cloned into an HpaI site in UNC6.W to generate the chimeric vectors UNC6.EHAhFIX.

ELISA is used to test blood samples for human factor IX levels, using a sandwich ELISA taking advantage of the relative specificity of the A1 antibody for the threonine-148 dimorphism of human factor IX. A monoclonal human antibody (Hematologic Technologies, Inc., Essex Junction, Vt., United States of America) and mouse anti-human factor IX monoclonal antibody A1 are used as the detecting antibody. FIX antigen levels are calculated using a human factor-IX standard curve generated from purified recombinant Thr148 factor IX produced in human embryonic kidney 293 cells, and purified using batch adsorption to Q Sepharose. In addition, samples taken at days −7, 7, and 28 are analyzed by Western blot analysis for the presence of the vector-delivered factor IX (the threonine form), by using the A1 antibodies. Factor IX function is determined by one-stage clotting assay (factor IX-specific aPTT) assayed on the START 4 Coagulation Analyzer (Diagnostica Stago, Parsippany, N.J., United States of America) and whole blood clotting time assay.

All blood samples are analyzed for the emergence of inhibitory antibodies against human factor IX, using the Bethesda inhibitor assay. This assay is based on the factor IX-specific aPTT and determines a titer of inhibitor antibody based upon the dilution of study plasma that inhibits 50% FIX clotting activity from a normal plasma standard. In addition, all of the blood samples are analyzed for the presence of non-inhibitory anti-factor IX immunoglobulin subclasses.

### Example 23

### Creator HIV-1 Vector for Generating a Mutant Human Factor IX Transgenic Mouse

The vector expresses human factor IX missense mutant allele R333Q under CMV promoter. Hemophiliac patients carrying this mutation have nearly normal levels of serum factor IX; however, their clotting activity is less than 1%. Thus, human patients and hemophilic mice carrying such a mutation are less likely to develop inhibitory antibodies following a traditional protein replacement therapy, or a gene therapy protocol. Since the creator HIV-1 vector facilitates the generation and maintenance of a transgenic mouse strain, an IRES-GFP cassette is located downstream to the factor IX R333Q allele. The ability to detect GFP expression facilitates screening of the vector containing mice. A SIN HIV-1 vector containing a CMV promoter facilitates the screening of vector-containing mice. To generate the creator HIV-1 vector, a klenowed DNA fragment containing the R333Q allele is cloned into the HpaI site in pTK642 (a SIN HIV-1 vector containing a CMV promoter and an IRES GFP). Vector particles are generated by transient transfection, and vector titer determined by scoring GFP expression following serial dilution on 293T cells. The efficiency of the R333Q allele production is determined by ELISA and western analysis on conditioned media obtained from vector-transduced cells.

### Example 24

### Chimeric EIAV/HIV-1 vectors in Inducing an Effective Immune Response Following in vivo Gene Delivery

To characterize the ability of the chimeric EIAV/HIV-1 vector to induce humoral immune responses against the AAV-2 capsid, traditional HIV-1 vector and episomal chimeric EIAV/HIV-1 vector, expressing the AAV-2 capsid protein under the control of the CMV promoter, are IV injected into R333Q mice. The expression of the VP3 capsid protein of AAV-2 from this construct has been eliminated by mutating three methionine codons, M203, M211, and M235 into lysines. Blood samples are withdrawn by retro-orbital bleeding seven days prior to vector administration and at days 7, 14, 21, 28, 35, and 42 post-injection. To determine the development of inhibitory antibodies to the AAV-2 capsid, a total of 10⁸ genome units (GU) of AAV-2 vectors, expressing the GFP reporter gene under the control of a CMV promoter, are exposed to serial dilutions of mouse plasma, and the titer of inhibitory antibodies directed against the AAV-2 capsid is determined by their ability to inhibit 50% of 293T cells transduction in vitro. In addition, ELISA using plate-bound AAV-2 particles is employed to determine the concentration of the different antibody subclasses to the AAV-2 capsid protein.

To test the development of inhibitory antibodies in vivo, at day 42 post-transduction, groups of treated mice (injected earlier with lentiviral vectors expression the AAV-2 capsid) and untreated mice are injected IV with 10¹¹ GU of AAV-2 vectors, from which either the firefly luciferase or the hFIX cDNA is expressed under the control of the hAAT promoter (liver-specific). Blood samples are withdrawn from control mice and from mice injected with AAV-2 hFIX vectors at day 7 prior to AAV-2 vector injection, and at days 7, 14, 21, 42, and 70 post-injection. The level and function of hFIX in mouse serum is determined by ELISA and aPTT assays, respectively. In vivo luciferase expression in all mice injected with the AAV-2 luciferase vector, and in non-treated mice is determined by Xenogen imaging system (available from Xenogen Corporation, Alameda, Calif., United States of America).

A SpeI PCR fragment containing the triple-mutant AAV-2 capsid open reading frame is cloned into an XbaI site in pTK829 and pTK208 to generate the chimeric EIAV/HIV-1 vector pTK840 and the traditional HIV-1 vector pTK840H, respectively. The CMV promoter drives expression of the AAV-2 capsid gene from both vectors.

### Example 25

### Hemophilic Mouse Expressing Human factor IX R333Q Allele by HIV-1 Vector Transgene Delivery to Hemophilic Mouse Zygotes

VSV-G pseudotyped creator R333Q particles are generated by transient transfection. Following purification and concentration, the vector is resuspended to a concentration of 5×10⁹ IU/mL. Vector titer after centrifugation is determined by scoring GFP expression following serial dilutions on 293T cells. The vector is injected into the perivitelline space of zygotes obtained from superovulated KO mice.

Two cell embryos are transplanted into the oviducts of a foster mouse. Based on preliminary results, 85-95% of the injected embryos are expected to express the transgene, 35-40% of the transplanted embryos are expected to be born alive, and 80-90% of the live embryos are expected to express the transgene under the control of a CMV promoter. Overall, it is expected that 25-35% of the injected embryos develop into a transgene expressing adult. Neonates are screened for transgene expression by fluorescence imaging. All pups are genotyped by tail DNA PCR using primers directed to the WPRE (WP) and the R333Q sequence. The number of vector genome per mouse is determined by qPCR and Southern blot analysis. At about 8 weeks of age, the levels of R333Q expression are determined by ELISA. Three to 4 mice from which serum R333Q levels are higher than 500 ng/mL and contain a single copy of the HIV-1 vector are cross-bred to generate a colony of homozygous mice. The genotype status is determined by qPCR and Southern analysis.

### Example 26

### Comparison of the Features of the HIV-1 Vector-Derived Humanized, Hemophilic Mouse Model with Two Existing Hemophilic Mouse Models

The first model is based on the traditional factor IX KO mouse, which are prone to develop inhibitory antibodies. The second model is based on the humanized hemophilic mouse R333Q-hFIX, which was generated by the traditional embryonic stem cell knock-in technology. Thus, the parental mouse factor IX gene was replaced with the mutant human R333Q cDNA. As such, the endogenous factor IX promoter regulates the expression of the human R333Q cDNA in these mice. These mice do not develop inhibitory antibodies following intramuscular administration of adeno-associated viral vector from which a human factor IX cDNA as expressed under the control of a CMV promoter. Using HIV-1 (packaged with the traditional HIV-1 system), EIAV (packaged with the traditional EIAV system) and chimeric EIAV/HIV-1 (packaged with the traditional HIV-1 system) vectors to deliver the human factor IX cDNA under the control of either a CMV or the liver specific hAAT promoter, into the three strains of hemophilic mice, allows evaluation of the mouse models and the lentiviral vectors.

HIV-1, EIAV, and chimeric EIAV/HIV-1 vectors carrying the human factor IX under the control of a CMV or hAAT promoter are administered via IP injections into KO-FIX, R333Q-hFIX, and Lb-cR333Q mice. Each mouse receives a total of 2-3×10⁹ IU of the vectors. Blood samples are withdrawn by retro-orbital bleeding at day 7 pre-injection and at days 3, 7, 14, 21, 28, 42, 56, 70, and 91 post-injection for levels/function of human factor IX and development of human factor IX immune responses.

All the injected lentiviral vectors contain a human factor IX cDNA encoding a fully functional protein. This cDNA expresses the threonine form of the Ala148Thr human factor IX dimorphism. The R333Q-hFIX and the Lb-cR333Q mice express the alanine form of the human factor IX. Employing the unique antibody A1, which preferentially recognizes the threonine isoform, allows evaluation of the effectiveness of the proposed gene therapy protocols in the humanized hemophilic mice.

The HIV-1 vectors from which the human factor IX is expressed under the control of either a CMV or hAAt promoter has been developed, pTK757 (FIG. 1) and pTK759 (FIG. 1), respectively, and showed high levels of factor IX expression in culture (FIG. 31). To generate EIAV vectors expressing human factor IX, a klenowed BglII/BamHI DNA fragment containing the human factor IX cDNA is cloned into an HpaI site in the EIAV vector UNC6.W to generate the UNC6.WhFIX construct. Cloning of either a klenowed BglII/BamHI DNA fragment containing the CMV promoter or a klenowed BglII/Bam HI DNA fragment containing the hAAT promoter provides the EIAV vectors UNC6.WchFIx and UNC6.WAhFIX from which the human factor IX is expressed under control of the CMV or hAAT promoters, respectively. To generate chimeric EIAV vectors, a klenowed SacII/EcoNi DNA fragment containing the HIV-1 RRE and the human factor IX cDNA under the control of either a CMV or hAAT promoter is isolated from pTK757 or pTK759, respectively, and cloned into a HpaI site in UNC6.W to generate the chimeric vectors UNC6.EHcFIX and UNC6.EHaFIX.

All of the vectors are generated by transient three-plasmid transfection and purified/concentrated as described earlier (Cockrell, A. S., et al. (2003) Curr HIV Res 1:419-439). The concentration of HIV-1 and the chimeric EIAV/HIV-1 vector particles are determined by p24^{gag} ELISA and RT assay. The concentration of the EIAV vector particles is determined by RT assay. Infectious titers are determined by qPCR on DNA extracted from vector transduced 293T cells. ELISA is used to test blood samples from human factor IX levels. Samples are analyzed by Western blot analysis for the presence of the vector delivered factor IX (the threonine form) using the A1 antibodies. Factor IX function is determined by aPTT and whole blood clotting assay. All blood samples are analyzed for the emergence of inhibitory antibodies using the Bethesda assay. All blood samples are also analyzed for the presence of non-inhibitory anti-factor IX immunoglobulin subclasses.

### Example 27

### Biodistribution and Transduction Efficiency of the EIAV/HIV-1 Chimeric Vectors In Vivo

HIV-1 packaged with the traditional HIV-1 system or chimeric EIAV/HIV-1 vector (packaged with traditional HIV-1 system) expressing either luciferase or GFP reporter under the control of CMV or hAAT promoters are administered to Balb/c mice via IP injection. Additionally, HIV-1 or chimeric EIAV/HIV-1 vector Cre-GFP fusion protein, under the control of the CMV or hAAT promoters, are administered to the ROSA26 mice containing the LoxP-stop-LoxP-LacZ cassette. The use of a liver-specific promoter provides robust transgene expression in hepatocytes, which are the preferred target cells for correction of factor IX deficiency. However, to address safety issues, it is essential to evaluate EIAV/HIV-1 chimeric vector efficiency of transgene delivery to other target organs, necessitating the use of a vector from which the above reporter transgenes are expressed under the control of a ubiquitous promoter (CMV). The study is carried out in cohorts of 12 mice. Luciferase expression in all the relevant mice is evaluated at days 3, 7, 14, 21, 28, 42, 56, 70, and 91, using the Xenogen imaging system (Alameda, Calif., United States of America).

At day 7 post-injection, four mice from each group are sacrificed and several tissues (kidney, liver, spleen, brain, heart, gonads, bone marrow, lung) are harvested. Tissues from mice treated with the GFP or Cre-GFP are sectioned and analyzed by fluorescence microscopy, immunohistochemistry, or by LacZ staining. The cellular tropism of the HIV-1 and EIAV/HIV-1 chimeric vectors in the liver are determined by immunohistochemistry using antibodies directed to cellular-specific markers including albumin (hepatocytes), Ly71 (Kupffer cells), and CD146 (endothelial cells). Protein extract is prepared from tissues obtained from animals treated with luciferase reporter vectors, and quantitated for expression using luciferin substrate to detect luciferase. Further, DNA is extracted form liver tissue and assessed for vector copy number by quantitative PCR.

To analyze the effect of cell proliferation on non-integrating vectors, such as the EIAV/HIV-1 chimeric vector, and to establish that these vectors do not integrate into host cell genomes in vivo, partial hepatoectomy on four mice from each of the groups receiving different vectors is performed. This procedure induces liver cell proliferation. Consequently, it is expected that such large-scale cellular proliferation will lead to a loss of the EIAV/HIV-1 chimeric vector in the regenerating liver. Four animals from each group are subjected to partial hepatectomy at day 42. Liver tissue removed at the time of partial hepatectomy is assessed for vector copy number by quantitative PCR on extracted DNA.

### REFERENCES

The references listed below as well as all references cited in the specification are incorporated herein by reference to the extent that they supplement, explain, provide a background for or teach methodology, techniques and/or compounds employed herein.
Anderson (1992) Science 256:808.
Brown, P. O. (1997) Integration. J. M. Coffin, Huges, S. H., Varmus, H. E. (eds) Cold Spring Harbor Laboratory Press.
Bushman, F. D., and Craige, R. (1990) J Virol 64:5645-5648.
Cao, R. et al. (2002) Science 298:1039-1043.
Choo (ed) (1994) Methods In Molecular Biology Volume 33—In Situ Hybridization Protocols Humana Press Inc., New Jersey.
Crystal (1995) Science 270:404.
Cockrell, A. S., et al. (2003) Curr HIV Res 1:419-439.
Colicelli, J. and Goff, S. P. (1985) Cell 42:573-580.
Coligan (1991) Current Protocols in Immunology Wiley/Greene, N.Y.
Corbeau et al. (1996) Proc. Natl. Acad. Sci. USA 93:14070-14075.
Cossett et al., (1993) Virol., 193:385-395.
Danos, O., and Mulligan et al., (1988) PNAS 85:6460-6464.
Dranoff, G., et al., Proc. Natl. Acad. Sci., 90:3539-3543 (1993).
Finer, et al. (1994) Blood, 83:43-50.
Finco, T., and Baldwin, A. S., Jr. (1993) J. Biol. Chem. 268, 17676-17679
Fodor, et al. (1991) Science, 251: 767-777.
Frazier, D., et al. (1989) Blood 74(3):971-977.
Freshney (Culture of Animal Cells, a Manual of Basic Technique, third edition Wiley-Liss, New York (1994).
Goding (1986) Monoclonal Antibodies: Principles and Practice (2d ed.) Academic Press, New York, N.Y.
Goeddel, Gene expression Technology: Methods in Enzymology, page 185, Academic Press, San Diego, Calif. (1990).
Graham et al. (1977) J. Gen. Virol., 36:59-72.
Harlow and Lane (1989) Antibodies: A Laboratory Manual Cold Spring Harbor Press, N.Y.
Heinzel, et al. (1988) J. Virol., 62:3738.
Innis, M. A. and Gelfand, D. H. (1990) Optimization of PCRs. pp. 3-12 in: PCR Protocols (Innis, Gelfand, Sninsky and White, eds.) Academic Press, New York.
Jin, D. Y., et al. (2004) Blood 104(6):1733-1739.
Kafri, T., et al. (1999) J Virol 73:576-584.
Kohler and Milstein (1975) Nature 256: 495-497.
Kuchler, et al. (1977) Biochemical Methods in Cell Culture and Virology, Kuchler, R. J., Dowden, Hutchinson and Ross, Inc.
Malim, M. H., et al. (1989) Cell 58:205-214.
Mann, et al. (1983) Cell, 33:153-159.
Markowitz, et al. (1988) J. Virol., 62:1120-1124.
Merrifield (1963) J. Am. Chem. Soc. 85:2149-2154.
Miller (1992) Nature 357:455.
Miller, et al. (1986) Molec. and Cell. Biol. 6:2895-2902.
Miller, A. D., et al., Meth. in Enz., 217:581-599 (1993).
Mosbach, K., et al., Nature, 302:543-545.
Mulligan (1993) Science 260:926.
Otero, G. C., et al. (1998) J Virol 72:7593-7597.
Palva, I., et al., 1983, Gene 22:229-235.
Pear et al. (1993) PNAS 90:8392-8396.
Queen et al. (1989) Proc. Nat'l Acad. Sci. USA 86: 10029-10033.
Schmidt-Wolf and Schmidt-Wolf, 1994, Annals of Hematology 69; 273-279. Scopes (1982) Protein Purification: Principles and Practice Springer-Verlag New York.
Shaulian, E., A. Zauberman, D. Ginsberg, and M. Oren (1992) Mol. Cell. Biol. 12:5581-5592.
Sheldon, et al. (1993) Clinical Chemistry 39(4): 718-719.
Sherman, et al. (1982) Methods in Yeast Genetics, Cold Spring Harbor Laboratory.
Smith, K. J., et al. (1987) Blood 70(4):1006-1013.
Stewart and Young (1984) Solid Phase Peptide Synthesis, 2d. ed., Pierce Chemical Co.
Stites, et al. (eds.) Basic and Clinical Immunology (4th ed.) Lange Medical Publications, Los Altos, Calif.
Svarovskaia, E. S., et al. (2004) J Virol 78:3210-3222.
Tijssen (1993) Laboratory Techniques in biochemistry and molecular biology—hybridization with nucleic acid probes parts I and II, Elsevier, N.Y.
Xu, K., et al. (2001) Mol Ther 3:97-104
Yee, J. K., et al., (1994) Proc. Natl. Acad. Sci., 91:9654-9568.
Zapp, M. L., et al. (1991) Proc Natl Acad Sci USA 88:7734-7738.
Zufferey, R., Nagy, D., Mandel, R. J., Naldini, L. & Trono, D. (1997) Nat. Biotechnol. 15, 871-875
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,405,712
U.S. Pat. No. 4,650,764
U.S. Pat. No. 4,816,567
U.S. Pat. No. 4,861,719
U.S. Pat. No. 5,124,263
U.S. Pat. No. 5,399,346
U.S. Pat. No. 5,449,614

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method of producing chimeric vector particles, wherein a first retroviral vector is packaged into a second retroviral vector particle, the method comprising:
(a) cloning a nucleic acid sequence encoding a second retroviral vector cis element into the first retroviral vector to generate a chimeric vector; and
(b) transfecting a packaging cell line with said chimeric vector, wherein packaging cell line provides proteins for the retroviral vector to be packaged.

2. The method of claim 1, wherein the first retroviral vector comprises a lentivirus.

3. The method of claim 2, wherein the lentivirus is selected from the group consisting of FIV, EIAV, and MLV.

4. The method of claim 1, wherein the second retroviral vector cis element is selected from the group consisting of a RRE, an Env gene fragment from the region flanking the RRE, and cPPT.

5. The method of claim 4, wherein the Env gene fragment from the region flanking RRE is about 140 bp 5′ of the RRE and about 475 bp 3′ of the RRE.

6. A chimeric retroviral vector comprising sequences from at least two different retroviruses, wherein at least one of the sequences encodes a cis element that provides for cross-packaging of the chimeric retroviral vector in a viral particle.

7. The retroviral vector of claim 6, wherein the cis element is selected from the group consisting of a RRE, an Env gene fragment from the region flanking the RRE, and cPPT.

8. The retroviral vector of claim 7, wherein the Env gene fragment from the region flanking RRE is about 140 bp 5′ of the RRE and about 475 bp 3′ of the RRE.

9. The retroviral vector of claim 6, comprising, in 5′ to 3′ order:
(a) a 5′ long terminal repeat (LTR) from a first retrovirus;
(b) a sequence encoding a second retrovirus cis element; and
(c) a 3′ long terminal repeat (LTR) from the first retrovirus,
wherein the chimeric retroviral vector is capable of being packaged in a viral particle of the second retrovirus.

10. The retroviral vector of claim 9, wherein the first retrovirus is a non-HIV-1 retrovirus and the second retrovirus is a HIV-1 retrovirus.

11. The retroviral vector of claim 9, wherein each long terminal repeat region is derived from a retrovirus selected from the group selected from the group consisting of Murine Leukemia Virus, Mouse Mammary Tumor Virus, Murine Sarcoma Virus, Simian Immunodeficiency Virus, Human T Cell Leukemia Virus, Feline Immunodeficiency Virus, Feline Leukemia Virus, Bovine Leukemia Virus, and Mason-Pfizer-Monkey Virus.

12. The retroviral vector of claim 10, further comprising one or more HIV-1 envelope sequences oriented between the 5′ LTR and the 3′ LTR.

13. The retroviral vector of claim 12, wherein the one or more HIV-1 envelope sequences flank the RRE sequence 5′, 3′, or both 5′ and 3′.

14. The retroviral vector of claim 9, further comprising a HIV-1 cPPT sequence oriented between the 5′ LTR and 3′ LTR.

15. The retroviral vector of claim 14, wherein the cPPT sequence flanks the RRE sequence 3′.

16. The retroviral vector of claim 12, wherein the Env gene fragment from the region flanking HIV-1 RRE is about 140 bp 5′ of the RRE and about 475 bp 3′of the RRE.

17. The retroviral vector of claim 6, further comprising one or more coding sequences operably linked to a heterologous promoter.

18. The retroviral vector according to claim 17, wherein the coding sequences are selected from the group consisting of marker genes, therapeutic genes, antiviral genes, antitumor genes, cytokine genes, genes encoding antigens, and combinations thereof.

19. The retroviral vector according to claim 18, wherein said marker or therapeutic genes are selected from the group consisting of β-galactosidase gene, neomycin gene, puromycin gene, cytosine deaminase gene, secreted alkaline phosphatase gene, and combinations thereof.

20. The retroviral vector according to claim 9 or claim 19, comprising a heterologous promoter oriented 5′ to the 5′ LTR.

21. The retroviral vector according to claim 20, wherein the heterologous promoters are the same or different.

22. A recombinant retroviral particle comprising the retroviral vector according to claim 6, 9 or 17.

23. A composition comprising a recombinant retroviral particle according to claim 22 and a pharmaceutically acceptable carrier.

24. A retroviral provirus produced by infection of target cells with a recombinant retroviral particle according to claim 22.

25. mRNA of the retroviral provirus according to claim 24.

26. RNA of a retroviral vector according to claim 6.

27. A producer cell line for producing a viral particle, the producer cell comprising a retroviral vector and a construct coding for elements required for the retroviral vector to be packaged, wherein the retroviral vector comprising sequences from at least two different retroviruses, wherein at least one of the sequences encodes a cis element that provides for cross-packaging of the retroviral vector in a viral particle.

28. The producer cell line of claim 27, wherein the cis element is selected from the group consisting of a RRE, an Env gene fragment from the region flanking the RRE, and cPPT.

29. The producer cell line of claim 28, wherein the Env gene fragment from the region flanking RRE is about 140 bp 5′ of the RRE and about 475 bp 3′ of the RRE.

30. The producer cell line of claim 29, said retroviral vector comprising in 5′ to 3′ order:
(a) a 5′ long terminal repeat (LTR) from a first retrovirus;
(b) a sequence encoding a second retrovirus cis element; and
(c) a 3′ long terminal repeat (LTR) from the first retrovirus,
wherein the chimeric retroviral vector is capable of being packaged in a viral particle of the second retrovirus.

31. A retroviral vector kit comprising:
(a) a retroviral vector comprising sequences from at least two different retroviruses, wherein at least one of the sequences encodes a cis element that provides for cross-packaging of the retroviral vector in a viral particle; and
(b) a packaging cell line comprising at least one construct coding for proteins required for said retroviral vector to be packaged.

32. The retroviral vector kit of claim 31, wherein the cis element is selected from the group consisting of a RRE, an Env gene fragment from the region flanking the RRE, and cPPT.

33. The retroviral vector kit of claim 32, wherein the Env gene fragment from the region flanking RRE is about 140 bp 5′ of the RRE and about 475 bp 3′ of the RRE.

34. The retroviral vector kit of claim 31, said retroviral vector comprising in 5′ to 3′ order:
(a) a 5′ long terminal repeat (LTR) from a first retrovirus;
(b) a sequence encoding a second retrovirus cis element; and
(c) a 3′ long terminal repeat (LTR) from the first retrovirus,
wherein the chimeric retroviral vector is capable of being packaged in a viral particle of the second retrovirus.

35. The retroviral vector kit of claim 31, wherein the packaging cell line harbors retroviral or recombinant retroviral constructs coding for those retroviral proteins which are not encoded in said retroviral vector.

36. The retroviral vector kit of claim 31, wherein the packaging cell line is selected from the group consisting of SODk-1, WAN-1, or SODk-3.

37. A method for introducing homologous or heterologous nucleotide sequences into cells in an animal or cultured cells, the method comprising infecting the cells with a recombinant retroviral particle of claim 22.


SEQUENCE LISTING (12 sequences)

SEQ ID NO: 1 - DNA, 248 bp, Human immunodeficiency virus type 1
agagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 2 - DNA, 145 bp, Human immunodeficiency virus type 1
agtccgagatcttcagacctggaggaggagatatgagggacaattggagaagtgaattatataaatataaagtagtaaaaattgaaccattaggagtagcacccaccaaggcaaagagaagagtggtgcagagagaaaaaagagc

SEQ ID NO: 3 - DNA, 476 bp, Human immunodeficiency virus type 1
tggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacgga

SEQ ID NO: 4 - DNA, 28 bp, Human immunodeficiency virus type 1
gggtctgcatacaggagaaagagactgg

SEQ ID NO: 5 - DNA, 17 bp, Murine leukemia virus
gacgggggtctttcatt

SEQ ID NO: 6 - DNA, 18 bp, Murine leukemia virus
aatgaaagaccccacctg

SEQ ID NO: 7 - DNA, 20 bp, Human immunodeficiency virus type 1
gtggaaaaatctctagcagt

SEQ ID NO: 8 - DNA, 25 bp, Human immunodeficiency virus type 1
actggaagggctaatttggtccgaa

SEQ ID NO: 9 - DNA, 17 bp, Equine infectious anemia virus
gttcgagatcctacagt

SEQ ID NO: 10 - DNA, 19 bp, Equine infectious anemia virus
actgtggggtttttatgag

SEQ ID NO: 11 - DNA, 46 bp, Artificial
  Oligonucleotide designed for EIAV U3 att site
caaggggggaactggaagggctaatttggtccgaagggttttatac

SEQ ID NO: 12 - DNA, 46 bp, Artificial
  Oligonucleotide designed for EIAV U5 att site
gtctctagtttgtcgtggaaaaatctctagcagttggcgcccgaac